(19) United States
(12) Patent Application Publication
(10) Pub. No.: US 2014/0045241 A1
(43) Pub. Date: Feb. 13, 2014

(54) MICELLAR POLYSILOXANE ENZYME IMMOBILIZATION MATERIALS

(71) Applicants: Tracy Bucholz, St. Louis, MO (US); Tizah Anjeh, O'Fallon, MO (US)

(72) Inventors: Tracy Bucholz, St. Louis, MO (US); Tizah Anjeh, O'Fallon, MO (US)

(73) Assignee: Akermin, Inc., St. Louis, MO (US)

(21) Appl. No.: 14/003,652

(22) PCT Filed: Mar. 8, 2012
(86) PCT No.: PCT/US2012/028344

§ 371 Date: Oct. 22, 2013

Related U.S. Application Data
(60) Provisional application No. 61/450,424, filed on Mar. 8, 2011.

(51) Int. Cl. C12N 11/08; C12N 9/96

(52) U.S. Cl. CPC C12N 11/08; C12N 9/96
USPC 435/182; 435/188

(57) ABSTRACT

The present invention generally relates to immobilized enzymes for use in carbon capture and other systems; particularly, materials used to immobilize carbonic anhydrase are disclosed.

### FIELD OF THE INVENTION

The present invention generally relates to the immobilization of enzymes in various enzyme immobilization materials. Particularly, the enzyme immobilization materials described herein have mechanical, chemical, and temperature stability and sufficient permeability to substrates and products. Further, these materials are particularly appropriate for immobilizing carbonic anhydrase (CA).

### BACKGROUND OF THE INVENTION

In applied technology, more particularly in biotechnology, it is known that enzymes, enzyme-producing microorganisms, cells, or cell components can be fixed to certain carriers, particularly if they are used as biocatalysts. This process is known generally as immobilization. Since native enzymes are reduced in their activity by biological, chemical or physical effects during storage or in applications, there is a need to stabilize the enzymes in view of their high production costs. Through immobilization, the enzymes can be reused. After use, the enzymes are easy to remove from the reaction mixture. In this way, they can be used under a variety of processing conditions. Desirably, the substrate and reaction specificity and enzyme reactivity should not be lost as a result of immobilization.

Immobilized enzymes are used in particular in commercially-important biotechnological processes. Glucose isomerase, which converts glucose to fructose, is used in the food industry. Lipase can be used for transesterification of edible oils and immobilized enzymes are applied in the production of amino acids and in the splitting of penicillin G into 6-aminopenicillic acid. Immobilized enzyme and cell systems are also used in analysis, for example, in biosensors. The principle of analysis using immobilized systems is based on the reaction of a substrate to be determined by an immobilized enzyme, the changes in the concentrations of product, substrate and co-substrate being able to be followed, for example by several coupled methods (for example enzyme electrodes).

### SUMMARY OF THE INVENTION

Among the various aspects of the invention is an enzyme immobilized by entrapment and covalent bonding in a polymeric immobilization material. In many of these embodiments, the immobilization material is a micellar or inverted micellar polymer.

One aspect of the invention is an enzyme immobilized by entrapment in a polymeric micellar or inverted micellar immobilization material and by covalent attachment of the enzyme to the polymeric micellar or inverted micellar immobilization material. Another aspect is an enzyme having an amine group functionalized with a moiety, the moiety having the structure of formula 3

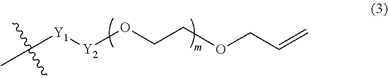

wherein m is an integer greater than 4, Y₁ is —CR₁₀R₁₁—, —O—, —S—, or —NR₁₂—, and Y₂ is —CCR₁₀R₁₁— or —C(O)—, R₁₀ and R₁₁ are independently hydrogen, alkyl, or aryl, and R₁₂ is hydrogen, alkyl, or aryl. A further aspect of the invention is an enzyme comprising an amine group functionalized with a moiety, the moiety having the structure of formula 4

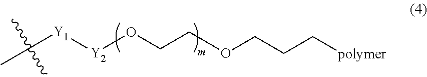

wherein m is an integer greater than 4, Y₁ is —CR₁₀R₁₁—, —O—, —S—, or —NR₁₂—, and Y₂ is —CCR₁₀R₁₁— or —C(O)—, R₁₀ and R₁₁ are independently hydrogen, alkyl, or aryl, and R₁₂ is hydrogen, alkyl, or aryl.

Yet a further aspect is an enzyme comprising an amine group functionalized with either a silsesquioxane; or a —C(O)—NH—R₁—Si(OH)₃ group wherein R₁ is C₁ to C₁₀ alkylene or C₁ to C₁₄ alkylene wherein one or more of the —CH₂— groups of the alkylene is replaced with an amine, an amide, or a carbonyl group.

Another aspect is an immobilized enzyme entrapped in a polymer comprising discrete hydrophilic regions and discrete hydrophobic regions wherein at least one of the hydrophilic regions has a diameter from about 4 nm to about 500 nm and at least one of the other hydrophilic regions has a diameter from about 1 μm to about 300 μm.

Yet another aspect is an immobilized enzyme comprising an enzyme aggregate comprising an enzyme crosslinked to another enzyme and entrapped within a non-ionic polymeric micellar or inverted micellar immobilization material.

A further aspect of the invention is an immobilized enzyme comprising an enzyme aggregate comprising an enzyme aggregated to another enzyme via non-covalent interactions and entrapped within a polymeric micellar or inverted micellar immobilization material comprising a polysulfone, a polycarbonate, a poly(vinylbenzyl chloride), a polysiloxane, or a combination thereof.

Another aspect is an immobilized enzyme comprising an enzyme crosslinked to a micellar or inverted micellar polymer, the micellar or inverted micellar polymer having the structure of formula 2

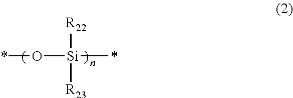

wherein R₂₂ and R₂₃ are independently hydrogen, alkyl, —O—(SiR₂₄R₂₅—O)ₘ—, —(CH₂)₃—O—(CH₂—CH₂—O)_{q}—CH₂—CH₂-acid, —(CH₂)₃—O—(CH₂—CH₂—O)_{q}CH₂—CH₂-base, or —(CH₂)₃—O—(CH₂—CH₂—O)_{q}—C₁-C₁₀ alkylene-enzyme, wherein one or more of the —CH₂— groups of the alkylene is replaced by —O—, —NH—, —S—, —C(O)—, aryl, or is substituted with a hydroxy group; R₂₄ and R₂₅ are independently alkyl; m, n and q are independently integers from 10 to 1000; provided that the average number of —(CH₂)₃—O—(CH₂—CH₂—O)_{q}—C₁-C₁₀ alkylene-enzyme groups per repeat unit is at least 0.03; wherein the enzyme of —(CH₂)₃—O—(CH₂—CH₂—O)_{q}—C₁-C₁₀ alkylene-enzyme is the enzyme crosslinked to a micellar or inverted micellar polymer.

### BRIEF DESCRIPTION OF THE DRAWINGS

FIG. 1 is an electron micrograph of a PHMS/PDMS polymer imaged at 1000× magnification using a 2 keV acceleration voltage. Scale bar=20 μm.

FIG. 2 is an electron micrograph of a PHMS/PDMS polymer imaged at 2500× magnification using a 2 keV acceleration voltage. Scale bar=10 μm.

FIG. 3 is an electron micrograph of a 2500× magnification of the PTA-stained cross-linked PHMS-g-PEG micellar polymer mixture (29 wt. % PEG overall) using 2 kV acceleration voltage. The solid material residing in recessions was determined to be tungsten. Scale bar=10 μm.

FIG. 4 is a X-ray analysis of different regions of the cross-linked PHMS-g-PEG micellar polymer mixture (29 wt. % PEG overall) sample. The bright, solid material in the recessed regions was confirmed to by tungsten.

FIG. 5 is an electron micrograph of a cross-linked PHMS-g-PEG micellar polymer mixture (29 wt. % PEG overall) stained with PTA and sputter coated with Au. The electron micrograph was taken at 5000× magnification using 15 keV acceleration voltage. Scale bar=5 μm.

FIG. 6 is an electron micrograph of a cross-linked PHMS-g-PEG micellar polymer mixture (29 wt. % PEG overall) stained with PTA and sputtered with Au. The electron micrograph was taken at 15000× magnification using 15 keV acceleration voltage. Scale bar=fpm.

FIG. 7 is an electron micrograph of a 2,500× magnification electron micrograph of cross-linked PHMS-g-PEG micellar polymer mixture (32 wt. % PEG overall; lower cross-link density) stained with PTA showing the large size of the recessed domains. Tungsten staining shows the existence of much smaller PEG domains within the larger recessions. Scale bar=10 μm

FIG. 8 is an electron micrograph of a 1,000× magnification electron micrograph of cross-linked PHMS-g-PEG micellar polymer mixture (32 wt. % PEG overall; lower cross-link density) using a backscattering electron (BSE) detector. BSE detection created a greater contrast between the heavy tungsten atoms and the polymer. Scale bar=20 μm.

FIG. 9 is an electron micrograph of a 2,500× magnification electron micrograph of cross-linked PHMS-g-PEG micellar polymer mixture (32 wt. % PEG overall; lower cross-link density) using a BSE) detector. Tungsten appears to aggregate near the center of the large domains. Smaller, discrete tungsten regions appear to be on the sub-micron scale. Scale bar=10 μm.

FIG. 10 is an electron micrograph of a 10,000× magnification image of cross-linked PHMS-g-PEG micellar polymer mixture (32 wt. % PEG overall; lower cross-link density) using a backscattering electron detector. The smaller domains stained with tungsten appear to range from tens to hundreds of nanometers. Scale bar=2 μm.

FIG. 11 is a series of SDS PAGE electrophoresis gels for the bioconjugation reaction of bCAII and mPEG-550 Da epoxide.

FIG. 12 is a series of SDS PAGE electrophoresis gels comparing the bioconjugation reaction of mPEG-5000 Da epoxide and bCAII at 30° C. pH 9.8 and 11.

FIG. 13 is a graph of the pH stat enzyme activity of bCAII upon exposure to methoxy-PEG 5000 Da-epoxide at various molar ratios at either pH 9.8 (blue) or 11 (black) (0.1M CO₃⁻²/HCO₃⁻) at 30° C. for 3 days. The assay conditions were as follows: 0.1 mg of enzyme, 400 sccm 10% CO₂, 50 mL 0.2M KHCO₃. An average of three 150 second runs was used for the determination of error bars.

FIG. 14 is a series of SDS PAGE electrophoresis gels of bCAII with varying ratios of functionalized mPEG 550 Da after reaction at room temperature at pH 9.8 (100 mM KHCO₃/50 mM K₂CO₃) for 24 hours. The three reactive groups on the PEG were the epoxide, 4-toluenesulfonyl (tosylate), and 4-nitrobenzyl (nosylate) groups.

FIG. 15 is a series of isoelectric focusing (IEF) gels of bCAII with varying ratios of functionalized mPEG 550 Da after reaction at room temperature at pH 9.8 (100 mM KHCO₃/50 mM K₂CO₃) for 24 hours. The three reactive groups on the PEG were the epoxide, tosylate, and nosylate moieties.

FIG. 16 is a series of SDS PAGE electrophoresis gels of bCAII at 40 molar excess of functionalized PEG 550 Da after reaction at 40° C. and 50° C. at pH 9.8 (100 mM KHCO₃/50 mM K₂CO₃) for 16 hours. The three reactive groups on the PEG were the epoxide, 4-toluenesulfonyl (tosylate), and 4-nitrobenzyl (nosylate) moieties.

FIG. 17 is a series of SDS PAGE electrophoresis gels of bCAII at a 40:1 ratio of various functionalized mPEGs after reaction at room temperature at pH 11 (0.1M Na2CO3) for 3 days. The three reactive groups on the mPEG were the epoxide, nosylate and tosylate.

FIG. 18 is a series of SDS PAGE electrophoresis gels of bioconjugation reaction between bCAII with various ratios of mPEG-5000 Da mesylate at 30° C. and pH 9.8 and 11.

FIG. 19 is a graph of pH stat enzyme activity of bCAII upon exposure to methoxy-PEG 5000 Da-mesylate at various molar ratios at either pH 9.8 (blue) or 11 (black) (0.1M CO₃⁻²/HCO₃⁻) at 30° C. for 3 days. The assay conditions were as follows: 0.1 mg of enzyme, 400 sccm 10% CO2, 50 mL 0.2M KHCO3. An average of three 150 second runs was used for the determination of error bars.

FIG. 20 is a graph of pH stat activity results from fractions collected in an SEC column after the bioconjugation reaction with mPEG-5000 Da mesylate at pH 11. The assay conditions were as follows: 0.1 mg of enzyme, 400 sccm 10% CO2, 50 mL 0.2M KHCO₃. An average of three 150 second runs was used for the determination of error bars.

FIG. 21 is a series of SDS PAGE electrophoresis gels of preparatory SEC column fractions of 60:1 mPEG 5000 Da-mesylate:bCAII reaction at pH 11, 30° C. for 3 days.

FIG. 22 is a series of SDS PAGE electrophoresis gels of bioconjugation reaction between bCAII with various ratios of mPEG-5000 Da tresylate at 30° C. for 24 hours at pH 11.

FIG. 23 is a graph of pH stat activity results of bCAII upon exposure to methoxy-PEG 5000 Da-tresylate at various molar ratios at either pH 11 (0.2M Na2CO3) at 30° C. for 1 day. The assay conditions were as follows: 0.1 mg of enzyme, 400 sccm 10% CO2, 50 mL 0.2M KHCO₃. An average of three 150 second runs was used for the determination of error bars.

FIG. 24 is a series of SDS PAGE electrophoresis gels of bCAII at various ratios of succinimide-functionalized PEG [both 550 Da (SPA) and 5000 Da (SVA)] after reaction at 4° C. or room temperature at pH 8.0 (100 mM phosphate buffer) for 16 hours.

FIG. 25 is a series of SDS PAGE electrophoresis gels of bCAII at various ratios of succinimide-functionalized PEG (5000 Da) after reaction at 4° C. at pH 8.0 or 7.5 (100 mM phosphate buffer) for 16 hours.

FIG. 26 is a graph of pH stat bCAII activity results upon exposure to methoxy-PEG-succinimides (550 Da, SPA=blue; 5000 Da, SVA=black) at various molar ratios at pH 8.0 (100 mM phosphate buffer) at 4° C. for 16 hours. The assay conditions were as follows: 0.1 mg of enzyme, 400 sccm 10% CO2, 50 mL 0.2M KHCO₃. An average of three 150 second runs was used for the determination of error bars.

FIG. 27 is a graph of pH stat activity results of unmodified bCAII (red) or PEI 1800 Da-functionalized bCAII (blue; PEI 1800 Da:EDC:bCAII=100:60:1) upon exposure to methoxy-PEG-succinimides (550 Da [SPA] or 5000 Da [SVA]) at various molar ratios at pH 7.5 (100 mM phosphate buffer) at room temperature for 16 hours. The assay conditions were as follows: 0.1 mg of enzyme, 400 sccm 10% CO2, 50 mL 0.2M KHCO₃. An average of three 150 second runs was used for the determination of error bars.

FIG. 28 is a graph of pH stat activity results of PEI 1800 Da-functionalized bCAII (PEI 1800 Da:EDC:bCAII=500:60:1) upon exposure to methoxy-PEG-succinimides (550 Da [SPA]=blue; 5000 Da [SVA]=green) at various molar ratios at pH 7.5 (100 mM phosphate buffer) at room temperature for 16 hours. The assay conditions were as follows: 0.1 mg of enzyme, 400 sccm 10% CO2, 50 mL 0.2M KHCO3. An average of three 150 second runs was used for the determination of error bars.

FIG. 29 is a series of SDS PAGE electrophoresis gels of hCAIV at various ratio of succinimide-functionalized PEG (550 Da; SPA) at pH 8.0 (100 mM phosphate buffer) at room temperature overnight.

FIG. 30 is a series of isoelectric focusing (IEF) gels of hCAIV at various ratios of succinimide-functionalized PEG (550 Da; SPA) at pH 8.0 (100 mM phosphate buffer) at room temperature overnight.

FIG. 31 is a graph of pH stat hCAIV activity results upon exposure to methoxy-PEG 550 Da-succinimide at various ratios at pH 8 (100 mM phosphate buffer) at room temperature for 16 hours. The two colors correspond to different sets of samples made at different times, hence why they both have control samples. The assay conditions were as follows: 0.1 mg of enzyme, 400 sccm of 10% CO₂, 50 mL of 0.2M KHCO₃ (pH=8.4). An average of three 150 second runs was used for the determination of error bars.

FIG. 32 is a series of SDS PAGE electrophoresis results of bCAII with various ratios of aldehyde-functionalized PEG 5000 Da at pH 7, 8, 9.2, or 10 (200 mM phosphate or carbonate buffer) at room temperature overnight.

FIG. 33 is a graph of pH stat activity results of bCAII upon exposure to various ratios of aldehyde-functionalized PEG 5000 Da at pH 7, 8, 9.2, or 10 (200 mM phosphate or carbonate buffer) at room temperature overnight.

FIG. 34 is a series of SDS PAGE electrophoresis gels of bCAII with various ratios of carbonyl imidazole-functionalized PEG 5000 Da at pH 10.0 or 10.5 (200 mM carbonate buffer) at room temperature for 3 days.

FIG. 35 is a series of SDS PAGE electrophoresis gels of bCAII with various EDC:CA molar ratios in the presence of excess polyethyleneimine (PEI) 1800 Da or polyethylene glycol (PEG) 2000 Da diamine.

FIG. 36 is a series of SDS PAGE electrophoresis gels of bCAII with various EDC:CA molar ratios in the presence of excess polyethyleneimine (PEI) 1800 Da or amino-dextran 10000 Da.

FIG. 37 is a series of UV chromatographs (280 nm) of the preparatory SEC column. Eluent is 50 mM tris-sulfate buffer (pH=7.5). Each tick mark represents 5 minutes. The elution limit of the column occurs at fraction #8. Chromatograph (a) is from native bCAII, where the enzyme peak occurs in fractions 14-18. Chromatograph (b) is from bCAII functionalized with 100-fold excess of amino-dextran 10000 Da at an EDC:bCAII ratio of 60:1, where the enzyme peak occurs in fractions 8-15.

FIG. 38 is a graph of a pH stat enzyme activity of bCAII with various EDC:CA molar ratios in the presence of excess polyethyleneimine (PEI) 1800 Da (in blue), amino-dextran 10000 Da (in black), or PEI 10000 Da (in green). The assay conditions were as follows: 0.1 mg of enzyme, 400 sccm 10% CO₂, 50 mL 0.2M KHCO₃ (pH=8.4). An average of three 150 second runs was used for the determination of error bars.

FIG. 39 is a series of isoelectric focusing (IEF) gels of small molecule amine-silanol (aminoethylaminopropyl-silanetriol, AEAPS) functionalized and unmodified bCAII. Running conditions: constant 200 V; 19 mA-2 mA; 110 minutes.

FIG. 40 is a series of isoelectric focusing (IEF) gels of both polymeric amine-silanols (silsesquioxanes, SSQs) functionalized and small molecule amine-silanol (AEAPS) functionalized as well as unmodified bCAII. Running conditions: constant 200 V; 19 mA-2 mA; 110 minutes.

FIG. 41 is a graph of pH stat activity comparison of both small molecule and polymeric amine-silanol modified bCAII vs. unmodified bCAII. The reaction conditions were as follows: 0.1 mg of enzyme, 400 sccm 10% CO₂, 50 mL 0.2M KHCO₃ (pH=8.4). An average of three 150 second runs was used for the determination of error bars.

FIG. 42 is a graph showing enzyme leaching results of AEAPS-modified vs. unmodified bCAII immobilized in a ˜100 micron thick PHMS-g-PEG micellar polymer coating (˜30 wt. % PEG overall) on functionalized ceramic. These samples are all stored in 1.9 molal KHCO₃/0.95 molal K₂CO₃ solution at various temperatures. The enzyme is introduced as a concentrated aqueous solution (˜300 mg/mL) at a 5.5 wt. % loading in the micellar polymer. The above data uses a determined transfer efficiency of 80% in estimating the initial amount of enzyme/polymer mixture used that is actually coated onto the ceramic packing.

FIG. 43 is a series attenuated total reflectance Fourier transform infrared spectroscopy (ATR FTIR) spectra of ceramic (general purpose alumina silicate) pretreated with hydrogen peroxide/ammonium hydroxide (blue line), then treated with hexamethylene diisocyanate (HDI) and dibutyldilauryltin catalyst at 60° C. (red line), and finally treated with disilanol-terminated PDMS 4200 Da and dibutyldilauryltin at 60° C. (green line). The red and green lines were shifted along the y-axis for clarity.

FIG. 44 is a series of ATR FTIR spectra of graphite pretreated with boiling concentrated nitric acid (blue line), then treated with hexamethylene diisocyanate (HDI) and dibutyldilauryltin catalyst at 60° C. (red line), and finally treated with disilanol-terminated PDMS 4200 Da and dibutyldilauryltin at 60° C. (green line). The red and green lines were shifted along the y-axis for clarity.

FIG. 45 is a series of FTIR spectra of aminoethylaminopropyltrimethoxysilane (AEAPTMS)-functionalized stainless steel and aminoethylaminopropylsilsesquioxane-methylsilsesquioxane copolymer (AEAPSSQ-MSSQ)-functionalized stainless steel as compared to blank (acid pickling only) stainless steel.

FIG. 46 is a graph of pH stat activity comparison of bCAII lifetime at 50° C. in 1M K₂CO₃ for enzyme free in solution vs. immobilized as a lyophilized powder at 4 wt. % in PHMS-g-PEG micellar polymer pellets (¼″ deep×⅛″ diameter), both normalized to their respective initial values. The reaction conditions were as follows: 400 sccm 10% CO₂, 50 mL 0.2M KHCO₃ (pH=8.4). An average of three 150 second runs was used for the determination of error bars.

FIG. 47 is a graph of pH stat activity comparison of bCAII lifetime at 60° C. in 3M K₂CO₃ for enzyme free in solution vs. immobilized as a lyophilized powder at 4 wt. % in PHMS-g-PEG micellar polymer pellets (¼″ deep×⅛″ diameter; 29 wt. % PEG), both normalized to their respective initial values. The reaction conditions were as follows: 400 sccm 10% CO₂, 50 mL 0.2M KHCO₃ (pH=8.4). An average of three 150 second runs was used for the determination of error bars.

FIG. 48 is a graph of pH stat activity assay of the lifetime of bCAII immobilized as a lyophilized powder at 2.3 wt. % in PHMS-g-PEG micellar polymer pellets (¼″ deep× 3/16″ diameter; 29 wt. % PEG), normalized to its respective initial value. The reaction conditions were as follows: 400 sccm 10% CO₂, 50 mL 0.2M KHCO₃ (pH=8.4). An average of three 150 second runs was used for the determination of error bars.

FIG. 49 is a schematic of the batch reactor analysis system.

FIG. 50 is a graph of a batch reactor analysis of bCAII immobilized in PHMS-g-PEG micellar polymer pellets at 1.3 wt. % enzyme loading. Activity values have been background subtracted.

FIG. 51 is a graph of a batch reactor analysis of bCAII immobilized within PHMS-g-PEG ˜60 μm thick micellar polymer coating at 5.7 wt. % enzyme loading on ceramic. Activities values have been background subtracted.

FIG. 52 is a graph of a batch reactor analysis of AEAPS-functionalized bCAII immobilized within PHMS-g-PEG ˜60 μm thick micellar polymer coating at 4.8 wt. % enzyme loading on ceramic. Activity values have been background subtracted.

FIG. 53 is a schematic of the absorber column and measurement locations in the closed loop reactor system.

FIG. 54 is a graph of a volume specific rate of CO₂ capture presented with time in the closed loop reactor with AEAPS-functionalized bCAII immobilized within PHMS-g-PEG ˜60 μm thick micellar polymer coating at 4.8 wt. % enzyme loading on ceramic. Activity gradient by regression analysis shows −16.2% per 100 hours.

FIG. 55 is a graph of a batch reactor analysis of AEAPS-functionalized bCAII immobilized within PHMS-g-PEG ˜60 μm thick micellar polymer coating at 4.8 wt. % enzyme loading on ceramic.

FIG. 56 is a graph of a batch reactor analysis of PEI-functionalized bCAII immobilized within PHMS-g-PEG ˜60 μm thick micellar polymer coating at 5.7 wt. % enzyme loading on ceramic. Activity values have been background subtracted.

FIG. 57 is a series of isoelectric focusing (IEF) gels of N-acryloxysuccinimide-modified Novozymes CA as well as unmodified Novozymes CA under the conditions of: constant 200 V; 19 mA-2 mA; 110 minutes.

FIG. 58 is a graph of the pH stat activity comparison of various vinyl-functionalized Novozymes CA as compared to an unmodified enzyme control. The reaction conditions were as follows: 5 μg of enzyme, 1000 sccm 10% CO₂, 50 mL 30 mM tris(hydroxymethyl)methylamine (Tris) buffer (pH=8.6).

FIG. 59 is a graph of the percent enzyme retention results of allyl PEG-modified vs. unmodified Novozymes CA immobilized in a ˜55 micron thick PHMS-g-PEG micellar polymer coating (˜30 wt % PEG overall) on functionalized ceramic. Samples stored in 2M KHCO₃/1M K₂CO₃ solution at various temperatures. The enzyme was introduced as a concentrated aqueous solution (˜200 mg/mL) at a 6.8 wt % loading in the micellar polymer.

FIG. 60 is a schematic of the Single Pass Reactor (SPR) system.

FIG. 61 is a graph of the retained activity versus time for Novozymes CA both free (soluble) and immobilized in PHMS-g-PEG micellar polymer stored at 70° C. in a carbonate/bicarbonate solution. The immobilized enzyme activity was analyzed using the SPRs, and the free enzyme activity was analyzed with a pH stat. The pH stat reaction conditions were: 5 μg of enzyme, 1000 sccm 10% CO₂, 50 mL 30 mM tris(hydroxymethyl)methylamine (Tris) buffer (pH=8.6).

FIG. 62 is a graph of the SPR data showing the CO₂ conversion as a function of film thickness for 17.3 wt % allyl-PEG CA loading in PHMS-g-PEG (60 wt % PEG)+21 wt % PQA. Testing conditions were: ˜50 mL of ceramic packing, 400 sccm of 15% CO₂ inlet, 20 mL/min of a 20 wt % carbonate solution at 25% conversion (˜0.8M KHCO₃/1.2M K₂CO₃), 1 psig, room temperature.

FIG. 63 is a CLR trace of immobilized Novozymes CA in a PHMS-g-PEG coating on Sulzer structured packing The testing conditions were: 200 mL/min liquid flow (0.8M KHCO₃/1.2M K₂CO₃ at pH 10), 4 SLPM of 15% CO₂, 3 psig absorber pressure, 35° C.

FIG. 64 is a graph of the SPR data showing the CO₂ conversion for 21 wt. % allyl-PEG CA loading in PHMS-g-PEG (60 wt % PEG)+21 wt. % PQA (˜35 μm film thickness). Testing conditions were: ˜50 mL of ceramic packing, 400 sccm of 15% CO₂ inlet, 20 mL/min of a 20 wt % carbonate solution at 25% conversion (˜0.8M KHCO₃/1.2M K₂CO₃), 1 psig, room temperature.

### DESCRIPTION OF THE INVENTION

The present invention is directed to enzymes in various enzyme immobilization materials that have mechanical, chemical, and temperature stability and sufficient permeability to substrates and products of reactions. The enzyme immobilization materials have sufficient mechanical strength to withstand reaction conditions where high flow rates of reactants can leach enzymes from materials having less mechanical strength. Further, the immobilization materials withstand the solvents and other reactants used in the reaction; for example, the materials will not dissolve or otherwise degrade in solvents or fluid systems used in the reactions. Also, the enzyme immobilization materials preferably have a glass transition temperature and physical properties that allow them to undergo many different fabrication methods including hot pressing, spray coating, dip coating, and printing. Furthermore, the enzyme immobilization materials have sufficient permeability to the enzyme substrate to enable the substrate to interact with the active site of the enzyme and the product to be released from the enzyme active site.

Some of the following enzyme immobilization materials have the properties described above.

One object of the invention is an enzyme immobilized by entrapment in polymeric micellar or inverted micellar immobilization material and by covalent attachment of the enzyme to the polymeric micellar or inverted micellar immobilization material.

### Biocatalysts

A biocatalyst having activity for catalyzing a desired reaction can be immobilized in the immobilization materials described herein. In particular, the biocatalyst is an enzyme, which is used to catalyze a desired reaction. Generally, naturally-occurring enzymes, man-made enzymes, artificial enzymes and modified naturally-occurring enzymes can be immobilized. In addition, engineered enzymes that have been engineered by natural or directed evolution can be used. Stated another way, an organic or inorganic molecule that mimics an enzyme's properties can be used in the present invention. The enzymes that can be immobilized are oxidoreductases, transferases, hydrolases, lyases, isomerases, ligases, or combinations thereof. Other enzymes that can be used can be obtained by commonly used recombinant genetic methods such as error-prone PCR and gene shuffling. Furthermore, other suitable enzymes may be obtained by the mining of DNA from various environments such as in soil.

Also, artificial ribozymes active for carbon dioxide sequestration and release are known and can sequester carbon dioxide by forming carbon-carbon double bonds. These ribozymes can also be immobilized in the immobilization materials described herein and used for the purpose of capture or sequestration of carbon dioxide.

The enzymes that can be immobilized in the immobilization materials described herein are lipases, glucose isomerases, nitrilases, glucose oxidases, proteases (e.g., pepsin), amylases (e.g., fungal amylase, maltogenic amylase), cellulases, lactases, esterases, carbohydrases, hemicellulases, pentosanases, xylanases, pullulanases, β-glucanases, acetolactate decarboxylases, β-glucosidases, glutaminases, penicillin acylases, chloroperoxidases, aspartic β-decarboxylases, cyclodextrin glycosyltransferases, subtilisins, aminoacylases, alcohol dehydrogenases, amino acid oxidases, phospholipases, ureases, cholesterases, desulfinases, lignin peroxidases, pectinases, oxidoreductases, dextranases, glucosidases, galactosidases, glucoamylases, maltases, sucrases, invertases, naringanases, bromelain, ficin, papain, pepsins, peptidases, chymosin, thermolysins, trypsins, triglyceridases, pregastric esterases, phosphatases, phytases, amidases, glutaminases, lysozyme, catalases, dehydrogenases, peroxidases, lyases, fumarases, histadases, aminotransferases, ligases, cyclases, racemases, mutases, oxidases, reductases, ligninases, laccases, chloroperoxidases, haloperoxidases, hydrogenases, nitrogenases, oxynitrilases (mandelonitrile lyases), or combinations thereof.

More preferably, the enzyme immobilized is a carbonic anhydrase. The carbonic anhydrase (CA) used in the systems described herein catalyzes the conversion of carbon dioxide to bicarbonate ions and protons and the conversion of bicarbonate ions and protons to carbon dioxide. CA represents a family of structurally and genetically diverse enzymes that arose independently from different precursors as a result of convergent evolution (Tripp, B. C., Smith, K., & Ferry, J. G. (2001). Carbonic Anhydrase: New Insights for an Ancient Enzyme. Journal of Biological Chemistry, 276 (52), 48615-48618) (Elluche, S., & Pöggeler, S. (2010). Carbonic Anhydrases in Fungi. Microbiology, 156, 23-29). The various CA enzymes have been organized into five unrelated structural classes (e.g., alpha, beta, gamma, delta, and epsilon) which share no DNA sequence similarity and differ in protein structure and active site architecture. Despite these structural differences, the active sites of all classes of CA enzymes function with a single divalent metal cofactor which is essential for catalysis (Tripp, B. C., Smith, K., & Ferry, J. G. (2001). Carbonic Anhydrase: New Insights for an Ancient Enzyme. Journal of Biological Chemistry, 276 (52), 48615-48618). The most common metal cofactor in CA enzymes is zinc.

The α-class of CA is the predominant form expressed in mammals, and is the best characterized of all the CA classes. There are at least 16 α-CA or CA-related enzymes (Supuran, C. T. (2008). Carbonic Anhydrases—An Overview. Current Pharmaceutical Design, 14, 603-614) found in animals, as well as 6 forms found in bacteria. The β-class of CAs are found in green plants, blue-green algae, and bacteria (Zimmerman, S. A., & Ferry, J. G. (2008). The β and γ Classes of Carbonic Anhydrases. Current Pharmaceutical Design, 14, 716-721) (Rowlett, R. S. (2010). Structure and Catalytic Mechanism of the β-Carbonic Anhydrases. Biochimica et Biophysica Acta, 1804, 362-373). The γ-class is found in bacteria and an example would be the CA from Methanosarcina thermophila (CAM) (Zimmerman, S. A., & Ferry, J. G. (2008). The β and γ Classes of Carbonic Anhydrases. Current Pharmaceutical Design, 14, 716-721). The CAM gene has been cloned into E. coli and is expressed as the Zn-containing form (Alber, B. E., & Ferry, J. G. (1996). Characterization of Heterologously Produced Carbonic Anhydrase from Methanosarcina thermophila. Journal of Bacteriology (June), 3270-3274), but it is more active as the Fe-, Cd-, or Co-form. The δ-class can be found in the marine diatom Thalassiosira weissflogii (Zimmerman, S. A., & Ferry, J. G. (2008). The β and γ Classes of Carbonic Anhydrases. Current Pharmaceutical Design, 14, 716-721). This example protein is a dimer, with a monomeric molecular weight of 27 kD. The protein will bind Zn-, but Fe- and/or Cd-predominates in vivo. Likewise, the ζ-class is also found in the marine diatom Thalassiosira weissflogii (Zimmerman, S. A., & Ferry, J. G. (2008). The β and γ Classes of Carbonic Anhydrases. Current Pharmaceutical Design, 14, 716-721). The protein is also a dimer with a molecular weight of 50-60 kD. The catalytic properties of these two classes have not been characterized.

The mammalian CA enzymes are divided into four broad subgroups depending on the tissue or cellular compartment location (e.g., cytosolic, mitochondrial, secreted, and membrane-associated). The CAII and CAIV enzymes are the most catalytically efficient of all the CAs characterized, demonstrating rates of catalysis that are near the theoretical limit for diffusion-controlled rates. CA IV demonstrates particularly high temperature stability, which is believed to result from the presence of two disulfide linkages in the enzyme.

Mammalian carbonic anhydrase, plant carbonic anhydrase, or microbial carbonic anhydrase; preferably, bovine carbonic anhydrase II or human carbonic anhydrase IV is used. Human carbonic anhydrase IV is available from William S. Sly at St. Louis University and is described in more detail in the following references: T. Okuyama, S Sato, X. L. Zhu, A. Waheed, and W. S. Sly, Human carbonic anhydrase IV: cDNA cloning, sequence comparison, and expression in COS cell membranes, Proc. Natl. Acad. Sci. USA 1992, 89(4), 1315-1319 and T. Stams, S. K. Nair, T. Okuyama, A. Waheed, W. S. Sly, D. W. Christianson, Crystal structure of the secretory form of membrane-associated human carbonic anhydrase IV at 2.8-Å resolution, Proc. Natl. Acad. Sci. USA 1996, 93, 13589-13594.

Compounds that mimic the active site of carbonic anhydrase can also be used. For example, various metal complexes have been used to mimic the carbonic anhydrase active site. For example, [Zn₂(3,6,9,12,20,23,26,29-octaazatricyclo[29.3.1.1^{14,18}]hexatriaconta-1(34), 14,16,18(36),31(35),32-hexaene)(CO₃)]Br₂.7H₂O and [Zn₂(3,6,9,12,20,23,26,29-octaazatricyclo[29.3.1.1^{14,18}]hexatriaconta-1(34), 14,16,18(36),31(35),32-hexaene)(CO₃)]Br₂.0.5CH₃COCH₃.5H₂O (See Qi et al., Inorganic Chemistry Communications 2008, 11, 929-934). Also used as a mimic for carbonic anhydrase was [tris(2-benzimidazolylmethyl)amineZn(OH)₂]²⁺, [tris(2-benzimidazolyl)amineZn(OH)₂](ClO₄)₂, and [tris(hydroxy-2-benzimidazolylmethyl)amineZn(OH)]ClO₄.1.5H₂O were also used to hydrate CO₂. (See Nakata et al., The Chemistry Letters, 1997, 991-992 and Echizen et al., Journal of Inorganic Biochemistry 2004, 98, 1347-1360)

Preferably, the carbonic anhydrase can be immobilized. The carbonic anhydrases and immobilized carbonic anhydrases disclosed herein can be used to catalyze the conversion of carbon dioxide to carbonic acid (e.g., bicarbonate and a proton in aqueous solution) or the conversion of bicarbonate and a proton to carbon dioxide.

Also, the enzyme can be an enzyme aggregate comprising an enzyme crosslinked to another enzyme. This crosslinking can be carried out during the immobilization of the enzymes by adding a carboxylic acid activating agent (e.g., EDC) to the mixture during the immobilization of the enzyme in the polymeric immobilization material. Further, these enzyme aggregates can form via non-covalent interactions. Various enzyme aggregates can be immobilized in the polymeric micellar or inverted micellar immobilization materials described herein.

The enzyme can also be modified to contain an allyl group. An aspect of the invention is an enzyme having an amine group functionalized with a moiety, the moiety having the structure of formula 3

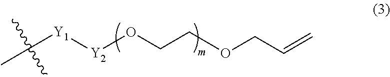

wherein m is an integer greater than 4, Y₁ is —CR₁₀R₁₁—, —O—, —S—, or —NR₁₂—, and Y₂ is —CCR₁₀R₁₁— or —C(O)—, R₁₀ and R₁₁ are independently hydrogen, alkyl, or aryl, and R₁₂ is hydrogen, alkyl, or aryl. Preferably, m is an integer greater than 10, Y₁ is —CH₂— or —NH— and Y₂ is —CH₂— or —C(O)—. Preferably, m is an integer from 10 to 1200 or from 30 to 1200. Also, Y₁ can be —CH₂— and Y₂ can be —CH₂—. The enzyme can have more than one amine group functionalized with the moiety, the moiety of formula 3.

The polysiloxane can be a crosslinked polysiloxane reaction product derived from a crosslinking reaction mixture of a grafted polysiloxane and an enzyme modified with an allyl group; wherein the grafted polysiloxane is derived from a grafting reaction mixture comprising a polysiloxane containing silicon hydride (Si—H) bonds and an enzyme modified with an allyl group. Preferably, the enzyme modified with an allyl group is an enzyme having an amine group functionalized with a moiety, the moiety having the structure of formula 3.

Preferably, the enzyme having an amine group functionalized with a moiety, the moiety having the structure of formula 3 is a carbonic anhydrase.

The enzyme can further comprise an amine group functionalized with a moiety, the moiety having the structure of formula 4

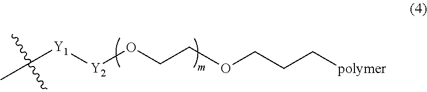

wherein m is an integer greater than 4, Y₁ is —CR₁₀R₁₁—, —O—, —S—, or —NR₁₂—, Y₂ is —CCR₁₀R₁₁— or —C(O)—, R₁₀ and R₁₁ are independently hydrogen, alkyl, or aryl, and R₁₂ is hydrogen, alkyl, or aryl. Preferably, m is an integer greater than 10, Y₁ is —CH₂— or —NH— and Y₂ is —CH₂— or —C(O)—. More preferably, m is an integer from 10 to 1200 or from 30 to 1200. Also, Y₁ can be —CH₂— and Y₂ can be —CH₂—. The enzyme can have more than one amine group functionalized with the moiety, the moiety of formula 4.

Preferably, the enzyme having an amine group functionalized with a moiety, the moiety having the structure of formula 4 is a carbonic anhydrase.

In formula 4, the polymer is a micellar or inverted micellar polymer. Preferably, the polymer has discrete hydrophobic and hydrophilic regions in the solid state. More preferably, the polymer is a polysulfone, a polycarbonate, a poly(vinylbenzyl chloride), a polysiloxane, or a combination thereof. In particular, the polymer is a polysiloxane.

The enzyme having an amine group functionalized with a moiety, the moiety having the structure of formula 4 includes a polymer, the polymer having the structure of formula 2

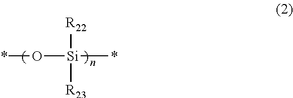

wherein R₂₂ and R₂₃ are independently hydrogen, alkyl, —O—(SiR₂₄R₂₅—O)ₘ—, —(CH₂)₃—O—(CH₂—CH₂—O)_{q}—CH₂—CH₂-acid, —(CH₂)₃—O—(CH₂—CH₂—O)_{q}—CH₂—CH₂-base, or —(CH₂)₃—O—(CH₂—CH₂—O)_{q}—C₁-C₁₀ alkylene-enzyme, wherein one or more of the —CH₂— groups of the alkylene is replaced by —O—, —NH—, —S—, —C(O)—, aryl, or is substituted with a hydroxy group; R₂₄ and R₂₅ are independently alkyl; m, n and q are independently integers from 10 to 1000; provided that the average number of —(CH₂)₃—O—(CH₂—CH₂—O)_{q-}C₁-C₁₀ alkylene-enzyme groups per repeat unit is at least 0.03; wherein the enzyme of —(CH₂)₃—O—(CH₂—CH₂—O)_{q}—C₁-C₁₀ alkylene-enzyme can be the enzyme comprising the amine group functionalized with the moiety, the moiety having the structure of formula 4.

The average number of —C₁ to C₁₀ alkylene-enzyme groups per repeat unit can be at least 0.05. 0.1, 0.15, 0.2, 0.25, 0.3, 0.35, or 0.4.

The enzymes described above can be used for these immobilized enzymes.

Preferably, the enzyme is a carbonic anhydrase; more preferably, the carbonic anhydrase is an alpha-carbonic anhydrase, a beta-carbonic anhydrase, a gamma-carbonic anhydrase, a delta-carbonic anhydrase, or an epsilon-carbonic anhydrase. The carbonic anhydrase is an alpha-carbonic anhydrase and further is a cytosolic carbonic anhydrase, a mitochondrial carbonic anhydrase, a secreted carbonic anhydrase, or a membrane-associated carbonic anhydrase.

More preferably, the carbonic anhydrase is a mammalian carbonic anhydrase, a plant carbonic anhydrase, or a microbial carbonic anhydrase; most preferably, a bovine carbonic anhydrase II or a human carbonic anhydrase IV.

An immobilized enzyme of the invention can comprise an enzyme covalently attached to the micellar or inverted micellar polymeric immobilization material through an amine, carboxylate, sulfhydryl, or hydroxyl group of the enzyme.

An immobilized enzyme can comprise an enzyme covalently attached to the micellar or inverted micellar polymeric immobilization material through the amine group of the enzyme.

There are several PEG derivatives that can react with the amine group including PEG epoxides, PEG sulfonate esters, PEG succinimide esters, PEG carbonyl imidazoles, and PEG aldehydes.

An immobilized enzyme can comprise an enzyme covalently attached to the micellar or inverted micellar polymeric immobilization material through the amine group of the enzyme wherein the amine group of the enzyme reacts with a polyethylene glycol group of the polymeric immobilization material to form an amine linking group having an adjacent —CH₂—CH(OH)—CH₂— or alkylene group.

PEG epoxides are commercially available or can be synthesized using a modified literature procedure (Kaul, G.; Amiji, M., Pharmaceutical Research 2002, 19, 1061-1067).

The bioconjugation reaction of PEG-550 Da epoxide with bovine carbonic anhydrase II (bCAII) occurred at a very slow rate. After collecting results at different pHs and temperatures, it was determined that the best conditions for the bioconjugation reaction between PEG epoxides and bCAII was high pH (e.g., pH 11) and high temperature (e.g., 35-40° C.).

Bioconjugation with a higher molecular weight PEG epoxide was detected on the SDS PAGE gel more easily than lower molecular weight PEG derivatives.

Nosylate (4-nitrophenylsulfonyl) and tosylate (4-toluenesulfonyl) functional groups can be used to attach polyethylene glycol (PEG) of the polysiloxane micellar and inverted micellar polymers to amine groups of enzymes. The nosylate and tosylate groups are well-known and effective leaving groups and react with the amine groups of the enzyme. A modified procedure was used for larger molecular weight PEGs because of a difference in solubility. Various derivatives can be synthesized including the methoxyPEG-550 Da nosylate and methoxyPEG-550 Da tosylate, methoxyPEG-1900 Da tosylate, and methoxyPEG-5000 Da tosylate. This methoxyPEG nosylate or methoxyPEG tosylate can then undergo a bioconjugation reaction with an enzyme as described in more detail in the examples.

Low molecular weight PEG nosylate, PEG tosylate, and PEG epoxide (e.g., PEG-550 Da nosylate, PEG-550 Da tosylate, and PEG 550 Da epoxide) were reacted with bCAII at various ratios for from about 18 to about 30 hours, preferably 24 hours; the reaction took place at pH 7 to pH 10, preferably at pH 9. The SDA PAGE data could not distinguish whether a reaction occurred since the lower molecular weight PEG derivatives did not add sufficient weight to move the PEGylated enzyme on the gel relative to unPEGylated enzyme. Thus, higher molecular weight PEG nosylate, PEG tosylate, and PEG epoxide were prepared.

The low molecular weight PEG nosylate, PEG tosylate, and PEG epoxide (e.g., PEG-550 Da nosylate, PEG-550 Da tosylate, and PEG 550 Da epoxide) appeared to not react substantially with the enzyme. The literature reports that tosylates and nosylates require elevated temperatures to react with an enzyme. In particular, the reaction takes place at a reaction temperature of about 30° C. to about 60° C.; preferably, from about 40° C. to about 50° C.

Also reactions with the nosylates and tosylates can be carried out at a higher pH (e.g., pH 11) for a longer time (e.g., 3 days). While reactions did produce some PEGylated enzyme, the yield was low.

Additionally, methoxyPEG mesylates and methoxyPEG tresylates were prepared. The 5000 dalton methoxyPEG mesylate and methoxyPEG tresylate were used to modify the enzyme. The reaction conditions and results for the bioconjugation of mPEG-5000 Da mesylate with bCAII are described in the examples.

Bioconjugation of the methoxyPEG-5000 Da mesylate to the enzyme was completed after about 3 days. The enzyme retained up to 75% of its initial activity after bioconjugation.

The bioconjugation reaction between bCAII and mPEG-5000 Da tresylate was more efficient than the bioconjugation reaction between bCAII and mPEG-5000 Da mesylate. The enzyme was almost completely PEGylated at a PEG tresylate:bCAII ratio of from about 40:1 to about 60:1. The enzyme retained up to about 50% of its initial catalytic activity after bioconjugation.

The pH, ionic strength of buffer, temperature, reaction times, and ratio of PEG to enzyme are factors that affect the enzyme activity when bioconjugating PEG tresylate to an enzyme. Also, the reaction of the PEG tresylate derivative with a micellar or inverted micellar polymer is necessary to complete the synthesis of the immobilized enzyme system.

The invention also includes an immobilized enzyme comprising an enzyme covalently attached to the micellar or inverted micellar polymeric immobilization material through the amine group of the enzyme wherein the amine group of the enzyme reacts with a polyethylene glycol group of the polymeric immobilization material to form an amide linking group.

Another functional group that can be used to tether the bCAII enzyme onto the polyethylene glycol (PEG) chain of the polymer is a succinimide ester. PEG-N-hydroxylsuccinimide (NHS) valerate (SVA) and PEG-NHS propionates (SPA) have been shown to undergo bioconjugation with enzyme and the carbonic anhydrase was completely PEGylated in most cases.

The reaction of the PEG succinimide ester with a primary amino group of the enzyme results in formation of an amide. PEG-NHS esters are well known for this transformation and are very reactive towards the amino groups on the enzyme.

A consideration for reaction conditions of the PEG-NHS esters with enzymes, particularly carbonic anhydrase, was minimization of hydrolysis of the PEG-succinimide ester. Hydrolysis was more facile for esters with short chain lengths between the reactive carboxyl and the last PEG ether group as shown in the following table.

### Hydrolysis Half-Lives of PEG NHS Ester at pH 8, 25° C.

| | | Half-life |
|---|---|---|
| PEG NHS Ester | Ester (Symbol) | (minutes) |
| PEG-O—(CH₂)₄—CO₂-NHS | Succinimidyl Valerate | 33.6 |
| | (SVA) | |
| PEG-O—CO₂-NHS | Succinimidyl Carbonate | 20.4 |
| | (SC) | |
| PEG-O—CH₂CH₂—CO₂-NHS | Succinimidyl Propionate | 16.5 |
| | (SPA) | |
| PEG-O—CH₂—CO₂-NHS | Succinimidyl | 0.75 |
| | Carboxymethyl (SCM) | |

When using PEG succinimidyl propionate ester (mPEG 550 Da-SPA) at PEG:CA ratios from about 5:1 to about 60:1, preferably, about 10:1 to about 60:1, the results showed that the CA was PEGylated to a greater extent with a higher PEG:CA ratio. These succinimide PEG derivatives are more reactive with CA than PEG epoxides and PEG sulfonate esters.

For reaction of PEG 5000 Da-SVA reactions with CA, the ratio of PEG:CA can be from about 2:1 to 20:1, preferably from about 5:1 to 20:1. Further, this reaction can take place from about 0° C. to about 10° C., preferably at about 4° C. The pH of the reaction can be from about 7 to about 9, preferably at about pH 7.5 to about 8.0. The reaction time can be from about 12 hours to about 20 hours; preferably about 16 hours.

At low PEG:CA ratios, the CA retained most of its catalytic activity, however as the PEG:CA ratio increased, the retention of catalytic activity of the CA decreased. Where there was no unmodified CA left in the sample, there was some retention of catalytic activity of the CA when the CA was bioconjugated with PEG 5000 Da-SVA.

The CA can be modified by attaching polymers containing multiple primary amine moieties (e.g., polyamines) to the surface of CA. By modifying CA in this way, the reaction of the PEG derivative is likely with an amine group attached to the polymer rather than an amine group of the CA and may help preserve the catalytic activity of CA after the bioconjugation reactions. To that end, the PEG-succinimide ester reactions described above were performed using CA that was functionalized with a polyamine, such as polyethyleneimine (PEI) having a molecular weight of 1800 Da. The polyamine was reacted with the carboxylic acid groups of CA using carbodiimide coupling with 1-ethyl-3-(3-dimethylaminopropyl) carbodiimide (EDC). For the polyamine functionalization of CA, the polyamine can be in 50-fold to 150-fold molar excess of the bCAII; and preferably, it is in 100-fold molar excess to the CA. The ratio of EDC to CA can be from about 40:1 to about 80:1, preferably, about 60:1. By modifying the CA with a polyamine, more of the catalytic activity of the CA is preserved when reacted with the PEG succinimide esters as compared to the same reaction when the CA was not modified with a polyamine. However, at higher ratios of PEG-succinimidyl ester to CA, this enhanced retention of catalytic activity is reduced.

The molecular weight and the ratio of polyamine to enzyme affect the number of primary amine groups on the CA surface. With more primary amine groups on the surface of the CA, the retention of enzyme activity is more likely to be increased because the PEG derivative can react with the amine group of the polyamine rather than an amine group that may affect the catalytic activity of the CA. For example, when the relative amount of polyamine to CA is higher, the enzyme activity is higher as compared to a lower relative amount of polyamine to CA where the enzyme activity is lower.

When a different form of CA, human carbonic anhydrase IV (hCAIV), was used for the reaction with methoxyPEG 500 Da-SPA, the hCAIV retained more of its catalytic activity than the same PEG derivative with bCAII. In fact, at the highest PEG ratios where all of the hCAIV reacted with at least one PEG chain, greater than 50% of the enzyme activity of the hCAIV remained.

PEG aldehydes react with the primary amino groups of proteins to form an imine, also known as a Schiff base. Schiff bases are not stable because they are susceptible to hydrolysis. To make the imines stable, a reduction to a secondary amine using reducing agents (e.g., sodium borohydride or sodium cyanoborohydride) is performed.

The reaction of PEG aldehydes with CA occurs at room temperature and at about pH 9 or higher. However, the yield of the resulting PEGylated CA was low. An accurate measurement of the activity retention of the enzyme was not available since most of the CA was not PEGylated.

Additionally contemplated is an immobilized enzyme comprising an enzyme covalently attached to the micellar or inverted micellar polymeric immobilization material through the amine group of the enzyme wherein the amine group of the enzyme reacts with a polyethylene glycol group of the polymeric immobilization material to form a carbamate linking group.

PEG carbonyl imidazole reacts with the primary amine groups of proteins to form a carbamate. However, reaction of CA and a PEG carbonyl imidazole derivative was not observed at the reaction conditions tested.

There are several other possible reactions that can be used to react with the amine groups of an enzyme, particularly CA. For example, PEG phenyl carbonates, PEG benzotriazole carbamates, and PEG carboxylic acids can react with an amine group of an enzyme to form a bioconjugate. A person of ordinary skill in the art would have known how to perform such reactions.

Also contemplated is an immobilized enzyme comprising an enzyme covalently attached to the micellar or inverted micellar polymeric immobilization material through the carboxylate group of the enzyme.

An immobilized enzyme can comprise an enzyme covalently attached to the micellar or inverted micellar polymeric immobilization material through the carboxylate group of the enzyme wherein the carboxylate group of the enzyme is activated with carbodiimide to enable the carboxylate group to react with an amine attached to the polymeric immobilization material.

Carbodiimide chemistry is a well-proven technique for coupling carboxylic acid groups of enzyme to amine-containing compounds to form stable amide bonds. For instance, water-soluble 1-ethyl-3-(3-dimethylaminopropyl)carbodiimide hydrochloride (EDC) is extensively used in protein conjugation. The incorporation of a catalytic amount of N-hydroxysulfosuccinimide (sulfo-NHS) stabilizes the activated carboxylate group, increasing the coupling efficiency between amines and activated carboxylates.

An immobilized enzyme can also comprise an enzyme covalently attached to the micellar or inverted micellar polymeric immobilization material through the sulfhydryl group of the enzyme.

Native enzymes typically do not contain free thiol groups that can react with maleimides, iodoacetamides, pyridyl disulfides, or vinyl sulfones. Free thiol groups are not in native proteins because the thiol groups are found in their oxidized disulfide form that stabilizes the tertiary structure of the protein. Because thiols can be selectively modified, they are usually an attractive site for genetic engineering. Modification of cysteine residues involves reaction of the thiol group with various derivatives. Thiols are more nucleophilic than primary amines, especially at less than pH 9 where amines are protonated. Consequently, cysteines often react faster than lysines, resulting in selective modification at the cysteine residue. A selective modification of an enzyme's thiol residue could also enhance the retention of the enzyme's catalytic activity.

The immobilized enzyme can also comprise an enzyme covalently attached to the micellar or inverted micellar polymeric immobilization material through the sulfhydryl group of the enzyme wherein the sulfhydryl group of the enzyme reacts with a polyethylene glycol group of the polymeric immobilization material to form a -maleimide-S— linking group.

A common approach to modification of cysteine residues is by reaction of the thiol of the enzyme with a maleimide moiety.

Maleimides react selectively with thiols via a Michael addition to the double bond at about pH 7.4 to give very stable conjugates. Under these bioconjugation conditions, lysines are unreactive. (Kim, Y.; Ho, S. O.; Gassman, N. R.; Korlann, Y.; Landorf, E. V.; Collart, F. R.; Weiss, S. Bioconjuate Chemistry 2008, 19, 786-791.)

Further, the immobilized enzyme can comprise an enzyme covalently attached to the micellar or inverted micellar polymeric immobilization material through the sulfhydryl group of the enzyme wherein the sulfhydryl group of the enzyme reacts with a polyethylene glycol group of the polymeric immobilization material to form an -acetamide-S— linking group.

The thiol group of CA can be converted to a thioether via SN2 type displacement of the iodo group in PEG iodoacetamide. These types of iodoalkanes are far more electrophilic than typical iodoalkanes due to the proximity to the carbonyl group to the iodine atom.

Additionally, the immobilized enzyme can comprise an enzyme covalently attached to the micellar or inverted micellar polymeric immobilization material through the sulfhydryl group of the enzyme wherein the sulfhydryl group of the enzyme reacts with a polyethylene glycol group of the polymeric immobilization material to form a disulfide linking group.

Another example of a reactive PEG that undergoes bioconjugation with cysteine residue is the PEG-ortho-pyridyldisulfide. This PEG reacts with thiols to yield a more stable dialkyl disulfide.

Also, the immobilized enzyme can comprise an enzyme covalently attached to the micellar or inverted micellar polymeric immobilization material through the sulfhydryl group of the enzyme wherein the sulfhydryl group of the enzyme reacts with a polyethylene glycol group of the polymeric immobilization material to form a sulfone-containing linking group.

PEG vinylsulfone reacts with thiols under neutral or mild alkaline conditions (pH 7.5 to 8.5) to form a thioether. (Masri, M. S.; Friedman, M. Journal of Protein Chemistry 1988, 7, 49-54.) Under higher pH conditions, amines will react with the PEG vinylsulfones.

In some embodiments, the immobilized enzyme comprises an enzyme covalently attached to the micellar or inverted micellar polymeric immobilization material through the hydroxyl group of the enzyme.

Modification of the hydroxyl group in the tyrosine residue of proteins is not as common as modification of amines, thiols, or carboxyl groups of the protein, however the modification is viable.

Also contemplated is an immobilized enzyme comprising an enzyme covalently attached to the micellar or inverted micellar polymeric immobilization material through the hydroxyl group of the enzyme wherein the hydroxyl group of the enzyme reacts with a polyethylene glycol group of the polymeric immobilization material to form a carbamate linking group.

Modification of the hydroxyl group in tyrosine is accomplished by its reaction with a PEG isocyanate that results in the formation of a carbamate.

Isocyanates are very reactive but also unstable. They decompose quite readily to give carbon dioxide and a primary amine. Isocyanates are also highly reactive towards amines, so they are not selective for a hydroxyl group. (Greenwald, R. B.; Pendri, A.; Bolikal, D. Journal of Organic Chemistry 1995, 60, 331-336.)

Further, the immobilized enzyme can comprise an enzyme covalently attached to the micellar or inverted micellar polymeric immobilization material through the hydroxyl group of the enzyme wherein the hydroxyl group of the enzyme reacts with a polyethylene glycol group of the polymeric immobilization material to form a carbonate linking group.

Tyrosine residues can also react with PEG nitrophenyl carbonate (PEG-NPC). However, this transformation is also nonselective. PEG NPC reacts with the hydroxyl group of tyrosine to give a carbonate and with an amine to yield a carbamate.

Functionalization of Enzyme with Reactive Groups that Couple with Micellar Polymer During Crosslinking

In another embodiment, the enzyme can comprise an amine group functionalized with either a silsesquioxane; or a —C(O)—NH—R₁—Si(OH)₃ group wherein R₁ is C₁ to C₁₀ alkylene or C₁ to C₁₄ alkylene wherein one or more of the —CH₂— groups of the alkylene is replaced with an amine, an amide, or a carbonyl group. In preferred embodiments, R₁ is ethylene, propylene or —(CH₂)₂—NH—(CH₂)₃—. In some of the embodiments, the silsesquioxane has the structure of formula 5

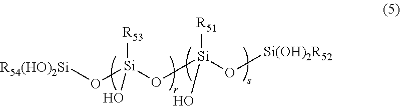

wherein R₅₁, R₅₂, and R₅₄ are independently C₂ to C₁₀ alkylene-NH₂ or C₂ to C₁₀ alkylene-NH₂ wherein one or more of the alkylene —CH₂— groups is replaced by an amine, an amide, or a carbonyl; R₅₃ is C₁ to C₁₀ alkyl; r is an integer from 0 to 20; preferably, 0 to 10; s is an integer from 1 to 20, preferably, 1 to 10; provided the —NH₂ group of at least one of R₅₁, R₅₂, or R₅₄ is coupled to a carboxylic acid group of the enzyme.

The enzyme preferably is a carbonic anhydrase; more preferably, the carbonic anhydrase is an alpha-carbonic anhydrase, a beta-carbonic anhydrase, a gamma-carbonic anhydrase, a delta-carbonic anhydrase, or an epsilon-carbonic anhydrase. In various preferred embodiments, the carbonic anhydrase is an alpha-carbonic anhydrase and further is a cytosolic carbonic anhydrase, a mitochondrial carbonic anhydrase, a secreted carbonic anhydrase, or a membrane-associated carbonic anhydrase. In particularly preferred embodiments, the carbonic anhydrase is a bovine carbonic anhydrase or a human carbonic anhydrase; preferably, a bovine carbonic anhydrase II or a human carbonic anhydrase IV. Also, the enzyme of this aspect of the invention has the structure where the —Si—OH group crosslinks with a polymer to form a covalent bond. In some these embodiments, the polymer is a micellar or inverted micellar polymer; preferably, the polymer has discrete hydrophobic and hydrophilic regions in the solid state. In various embodiments, the polymer is a polysulfone, a polycarbonate, a poly(vinylbenzyl chloride), a polysiloxane, or a combination thereof; preferably, the polymer is a polysiloxane.

The enzyme can also be covalently attached to a polymer having the structure of formula 1

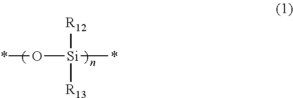

wherein R₁₂ and R₁₃ are independently hydrogen, alkyl, —O—(SiR₁₄R₁₅—O)ₘ—; or —C₁ to C₁₀ alkyl, —C₁ to C₁₀ alkylene-acid, —C₁ to C₁₀ alkylene-base, or —C₁ to C₁₀ alkylene-enzyme, wherein the —CH₃ group or one or more of the —CH₂— groups can be replaced by an amine group, an oxygen, an amide group, or a carbonyl group or wherein the —CH₃ group or one or more of the —CH₂— groups is substituted with a hydroxy, alkyl, or alkoxy; R₁₄ and R₁₅ are independently alkyl; m, n and q are independently integers from 10 to 1000; provided that the average number of —C₁ to C₁₀ alkylene-enzyme groups per repeat unit is at least 0.03. In many of these embodiments, R₁₂ and R₁₃ are hydrogen, methyl, —O—(Si(CH₃)₂—O—)ₘ—, —C₁ to C₁₀ alkyl, or —C₁ to C₁₀ alkylene-enzyme, wherein the —CH₃ group or one or more of the —CH₂— groups can be replaced by an amine group or an oxygen or wherein the —CH₃ group or one or more of the —CH₂— groups is substituted with a hydroxy or alkyl, wherein the enzyme of —C₁ to C₁₀ alkylene-enzyme is the enzyme comprising the amine group functionalized with silsesquioxane or a —C(O)—NH—R₁—Si(OH)₃ group. In other embodiments, R₁₂ and R₁₃ are independently hydrogen, alkyl, —O—(SiR₁₄R₁₅—O)ₘ—, —(CH₂)₃—O—(CH₂—CH₂—O)_{q}—CH₂—CH₂-enzyme; m, n and q are independently integers from 10 to 1000; provided that the average number of —(CH₂)₃—O—(CH₂—CH₂—O)_{q}CH₂—CH₂-enzyme groups per repeat unit is at least 0.03; wherein the enzyme of —(CH₂)₃—O—(CH₂—CH₂—O)_{q}—CH₂—CH₂-enzyme is the enzyme comprising the amine group functionalized with silsesquioxane or a —C(O)—NH—R₁—Si(OH)₃ group.

Since the polysiloxane-based micellar polymer mixtures have both silicon hydride (Si—H) and silanol (Si—OH) bonds, these polymers can be crosslinked into rubbery solids via several mechanisms. Thus, carbonic anhydrase can be functionalized with a variety of functional groups that can then react with the micellar silane polymer during crosslinking, including silanols (Si—OH), methoxysilanes (Si—OCH₃), and vinyl groups (CH₂═CH₂—R).

One such functionalization is depicted in the scheme below. This procedure uses a carbodiimide (e.g., 1-ethyl-3-(3-dimethylaminopropyl)carbodiimide (EDC)) to couple hydrophilic amine-silanols such as N-(2-aminoethyl)-3-aminopropylsilanetriol (AEAPS) or 3-aminopropylsilanetriol (APS) to the carboxylic acid groups of carbonic anhydrase.

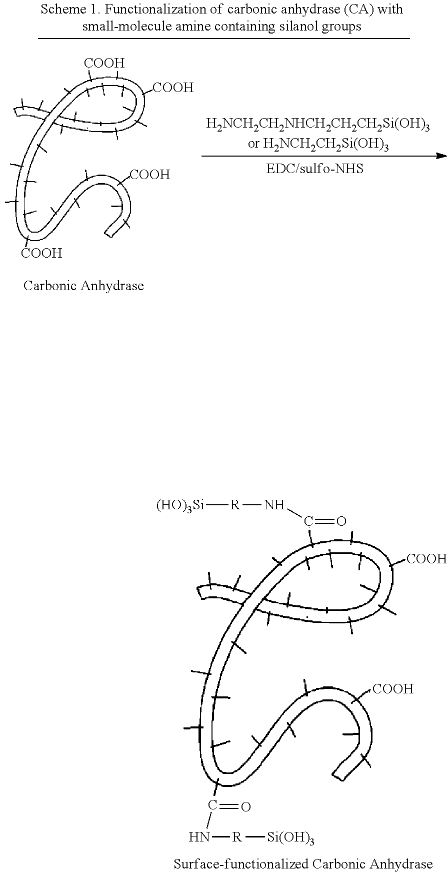

For this reaction, increasing the EDC:CA molar ratio increases the degree of modification. When EDC:CA ratios from about 5:1 to about 50:1 are used for the reaction, all of the enzyme is functionalized. When increasing the EDC:CA ratio within that range, the functionalized enzyme has an increasingly greater isoelectric point (pI values); this increase in pI shows a higher degree of functionality.

Alternatively, CA can be functionalized with amine-silanols known as silsesquioxanes that have a higher density of silanol groups to react with the micellar or inverted micellar polymer during crosslinking. Their structures are shown below.

### Aminoethylaminopropylsilsesquioxane-Methylsilsesquioxane Copolymer (AEAPSSQ-MSSQ, 65-75% Aminoethylaminopropyl Groups)

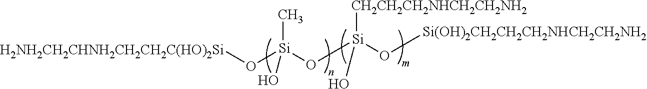

### Aminopropylsilsesquioxane-Methylsilsesquioxane Copolymer (A PSSQ-MSSQ 65-75% Aminopropyl Groups)

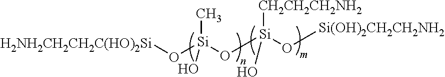

### Aminopropylsilsesquioxane Homopolymer (APSSQ)

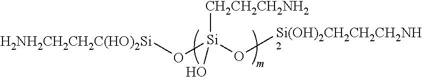

Modifying bCAII with either the small molecule amine-silanols (AEAPS) or the copolymer AEAPSSQ-MSSQ at differing EDC:CA ratios shows no appreciable loss in enzyme activity. Also, leaching studies of bCAII modified with AEAPS and unmodified bCAII immobilized in a micellar polymer coating on functionalized ceramic showed an improvement in physical enzyme retention at all storage temperatures investigated.

When the enzyme is functionalized with an allyl group as described above with reference to formulae 3 and 4, the enzyme can be functionalized with two or more allyl groups and the allyl-functionalized enzyme can be used to crosslink the polysiloxane immobilization material.

Preferably, the enzyme functionalized with two or more allyl groups used to crosslink the polysiloxane immobilization material is a carbonic anhydrase as described above.

### Enzyme Immobilization Materials

An enzyme can be immobilized by entrapment in a polymeric micellar or inverted micellar immobilization material and by covalent attachment of the enzyme to the polymeric micellar or inverted micellar immobilization material. This can be an enzyme, an enzyme mimic, an artificial ribozyme or a combination thereof.

For purposes of the present invention, an enzyme is “stabilized” if it either: (1) retains at least about 15% of its initial catalytic activity for at least about 30 days when continuously catalyzing a chemical transformation at room temperature; (2) retains at least about 15% of its initial catalytic activity for at least about 5 days when continuously catalyzing a chemical transformation at room temperature; (3) retains at least about 15% of its initial catalytic activity for at least about 5 days when being treated at temperatures from about 30° C. to about 100° C., (4) retains at least about 15% of its initial catalytic activity for at least about 5 days when continuously catalyzing a chemical transformation at room temperature and a pH from about 0 to about 13, (5) retains at least about 15% of its initial catalytic activity for at least about 5 days when continuously catalyzing a chemical transformation at room temperature in a non-polar solvent, an oil, an alcohol, acetonitrile, or a high ion concentration. Typically, a free enzyme in solution loses its catalytic activity within a few hours to a few days, whereas a properly immobilized and stabilized enzyme can retain its catalytic activity for at least about 5 days to about 1095 days (3 years).

Thus, the immobilization of the enzyme provides a significant advantage in stability. The retention of catalytic activity is defined as the enzyme having at least about 15% of its initial activity, which can be measured by a means that demonstrates enzyme-mediated generation of product such as chemiluminescence, electrochemical, potentiometric, mass spectrometry, spectrophotometric (i.e. UV-Vis), radiochemical, or fluorescence assay wherein the intensity of the property is measured at an initial time. The enzyme retains at least about 15% of its initial activity while the enzyme is continuously catalyzing a chemical transformation.

The retention of catalytic activity can be determine by measuring the carbon dioxide conversion to carbonic acid and the rate constant of that reaction measured in a single pass reactor as described in detail in example 32.

With respect to the stabilization of the enzyme, the enzyme immobilization material provides a chemical and/or mechanical barrier to prevent or impede enzyme denaturation. To this end, the enzyme immobilization material physically confines the enzyme, preventing the enzyme from unfolding. The process of unfolding an enzyme from a folded three-dimensional structure is one mechanism of enzyme denaturation.

The enzyme immobilization material stabilizes the enzyme so that the enzyme retains its catalytic activity for at least about 5 days to about 730 days (2 years).

Also, the immobilized enzyme retains at least about 75% of its initial catalytic activity for at least about 30, 45, 60, 75, 90, 105, 120, 150, 180, 210, 240, 270, 300, 330, 365, 400, 450, 500, 550, 600, 650, 700, 730, 800, 850, 900, 950, 1000, 1050, 1095 days or more. For example, the immobilized enzyme retains about 75% to about 95% of its initial catalytic activity for about 30 to about 1095 days, about 45 to about 1095 days, about 60 to about 1095 days, about 75 to about 1095 days, about 90 to about 1095 days, about 105 to about 1095 days, about 120 to about 1095 days, about 150 to about 1095 days, about 180 to about 1095 days, about 210 to about 1095 days, about 240 to about 1095 days, about 270 to about 1095 days, about 300 to about 1095 days, about 330 to about 1095 days, about 365 to about 1095 days, about 400 to about 1095 days, about 450 to about 1095 days, about 500 to about 1095 days, about 550 to about 1095 days, about 600 to about 1095 days, about 650 to about 1095 days, about 700 to about 1095 days, or about 730 to about 1095 days.

Further, the immobilized enzyme retains at least about 15, 20, 25, 30, 35, 40, 45, 50, 55, 60, 65, 70, 75, 80, 85, 90 or 95% or more of its initial catalytic activity for at least about 5, 7, 10, 15, 20, 25, 30, 45, 60, 75, 90, 105, 120, 150, 180, 210, 240, 270, 300, 330, 365, 400, 450, 500, 550, 600, 650, 700, 730, 800, 850, 900, 950, 1000, 1050, 1095 days or more. For example, the immobilized enzyme retains about 15 to about 95%, about 20 to about 95%, about 25 to about 95%, about 30 to about 95%, about 35 to about 95%, about 40 to about 95%, about 45 to about 95%, about 50 to about 95%, about 55 to about 95%, about 60 to about 95%, about 65 to about 95%, about 70 to about 95%, about 75 to about 95%, about 80 to about 95%, about 85 to about 95%, or about 90 to about 95% of its initial catalytic activity for about 5 to about 1095 days, about 7 to about 1095 days, about 10 to about 1095 days, about 15 to about 1095 days, about 20 to about 1095 days, about 25 to about 1095 days, about 30 to about 1095 days, about 45 to about 1095 days, about 60 to about 1095 days, about 75 to about 1095 days, about 90 to about 1095 days, about 105 to about 1095 days, about 120 to about 1095 days, about 150 to about 1095 days, about 180 to about 1095 days, about 210 to about 1095 days, about 240 to about 1095 days, about 270 to about 1095 days, about 300 to about 1095 days, about 330 to about 1095 days, about 365 to about 1095 days, about 400 to about 1095 days, about 450 to about 1095 days, about 500 to about 1095 days, about 550 to about 1095 days, about 600 to about 1095 days, about 650 to about 1095 days, about 700 to about 1095 days, or about 730 to about 1095 days.

An enzyme having greater temperature or pH stability may also retain at least about 75% of its initial catalytic activity for at least about 5 days when actively catalyzing a chemical transformation as described above.

For some immobilized enzymes, when exposed to a pH of less than about 2, less than about 3, less than about 4, or less than about 5, the stabilized enzyme retains at least about 15, 20, 25, 30, 35, 40, 45, 50, 55, 60, 65, 70, 75, 80, 85, 90 or 95% of its initial catalytic activity for at least about 5, 10, 15, 30, 40, 50, 60, 75, 90 days or more when continuously catalyzing a chemical transformation. For example, when exposed to a pH of less than about 2, less than about 3, less than about 4, or less than about 5, the stabilized enzyme retains about 15 to about 95%, about 20 to about 95%, about 25 to about 95%, about 30 to about 95%, about 35 to about 95%, about 40 to about 95%, about 45 to about 95%, about 50 to about 95%, about 55 to about 95%, about 60 to about 95%, about 65 to about 95%, about 70 to about 95%, about 75 to about 95%, about 80 to about 95%, about 85 to about 95%, or about 90 to about 95% of its initial catalytic activity for about 5 to 90 days, about 10 to 90 days, about 15 to 90 days, about 20 to 90 days, about 25 to 90 days, about 30 to 90 days, about 35 to 90 days, about 40 to 90 days, about 45 to 90 days, about 50 to 90 days, about 55 to 90 days, about 60 to 90 days, about 65 to 90 days, about 70 to 90 days, about 75 to 90 days, about 80 to 90 days, about 85 to 90 days when continuously catalyzing a chemical transformation.

When exposed to a pH of less than about 2, less than about 3, less than about 4, or less than about 5, the stabilized enzyme retains about 15 to about 95%, about 20 to about 95%, about 25 to about 95%, about 30 to about 95%, about 35 to about 95%, about 40 to about 95%, about 45 to about 95%, about 50 to about 95%, about 55 to about 95%, about 60 to about 95%, about 65 to about 95%, about 70 to about 95%, about 75 to about 95%, about 80 to about 95%, about 85 to about 95%, or about 90 to about 95% of its initial catalytic activity for at least about 5, 10, 15, 30, 40, 50, 60, 75, 90 days or more when continuously catalyzing a chemical transformation.

For some immobilized enzymes, when exposed to a pH of greater than about 9, greater than about 10, greater than about 11, or greater than about 12, the stabilized enzyme retains about 15 to about 95%, about 20 to about 95%, about 25 to about 95%, about 30 to about 95%, about 35 to about 95%, about 40 to about 95%, about 45 to about 95%, about 50 to about 95%, about 55 to about 95%, about 60 to about 95%, about 65 to about 95%, about 70 to about 95%, about 75 to about 95%, about 80 to about 95%, about 85 to about 95%, or about 90 to about 95% of its initial catalytic activity for about 5 to 90 days, about 10 to 90 days, about 15 to 90 days, about 20 to 90 days, about 25 to 90 days, about 30 to 90 days, about 35 to 90 days, about 40 to 90 days, about 45 to 90 days, about 50 to 90 days, about 55 to 90 days, about 60 to 90 days, about 65 to 90 days, about 70 to 90 days, about 75 to 90 days, about 80 to 90 days, about 85 to 90 days when continuously catalyzing a chemical transformation.

When exposed to an agent such as a nonpolar solvent, an oil, an alcohol, acetonitrile, a concentrated ionic solution, or combination thereof, the stabilized enzyme retains at least about 15, 20, 25, 30, 35, 40, 45, 50, 55, 60, 65, 70, 75, 80, 85, 90 or 95% of its initial catalytic activity for at least about 5, 10, 15, 30, 40, 50, 60, 75, 90 days or more when continuously catalyzing a chemical transformation. For example, when exposed to the agent, the stabilized enzyme retains about 10 to about 95%, about 15 to about 95%, about 20 to about 95%, about 25 to about 95%, about 30 to about 95%, about 35 to about 95%, about 40 to about 95%, about 45 to about 95%, about 50 to about 95%, about 55 to about 95%, about 60 to about 95%, about 65 to about 95%, about 70 to about 95%, about 75 to about 95%, about 80 to about 95%, about 85 to about 95%, or about 90 to about 95% of its initial catalytic activity for about 5 to 90 days, about 10 to 90 days, about 15 to 90 days, about 20 to 90 days, about 25 to 90 days, about 30 to 90 days, about 35 to 90 days, about 40 to 90 days, about 45 to 90 days, about 50 to 90 days, about 55 to 90 days, about 60 to 90 days, about 65 to 90 days, about 70 to 90 days, about 75 to 90 days, about 80 to 90 days, about 85 to 90 days when continuously catalyzing a chemical transformation.

In these instances, the concentration of the agent can be from about 1 wt. % to about 95 wt. %, 5 wt. % to about 95 wt. %, 10 wt. % to about 95 wt. %, 15 wt. % to about 95 wt. %, 20 wt. % to about 95 wt. %, 30 wt. % to about 95 wt. %, 40 wt. % to about 95 wt. %, 50 wt. % to about 95 wt. %.

An immobilized enzyme is an enzyme that is physically confined in a certain region of the enzyme immobilization material while retaining its catalytic activity. There are a variety of methods for enzyme immobilization, including carrier-binding, crosslinking and entrapping. Carrier-binding is the binding of enzymes to water-insoluble carriers. Crosslinking is the intermolecular crosslinking of enzymes by bifunctional or multifunctional reagents. Entrapping is incorporating enzymes into the lattices of a semipermeable material. The particular method of enzyme immobilization is not critically important, so long as the enzyme immobilization material (1) immobilizes the enzyme, and in some embodiments, (2) stabilizes the enzyme. In various embodiments, the enzyme immobilization material is also permeable to a compound smaller than the enzyme. An enzyme is adsorbed to an immobilization material when it adheres to the surface of the material by chemical or physical interactions. Further, an enzyme is immobilized by entrapment when the enzyme is contained within the immobilization material whether within a pocket of the material or not.

With reference to the immobilization material's permeability to various compounds that are smaller than an enzyme, the immobilization material allows the movement of a substrate or product compound through it so the substrate compound can contact the enzyme and the product compound can leave the enzyme. The immobilization material can be prepared in a manner such that it contains internal micelles, micellar pockets, channels, openings or a combination thereof, which allow the movement of the substrate compound throughout the immobilization material, but which constrain the enzyme to substantially the same space within the immobilization material. Such constraint allows the enzyme to retain its catalytic activity. In various preferred embodiments, the enzyme is confined to a space that is substantially the same size and shape as the enzyme, wherein the enzyme retains substantially all of its catalytic activity. The micelles, micellar pockets, channels, or openings have physical dimensions that satisfy the above requirements and depend on the size and shape of the specific enzyme to be immobilized.

The enzyme is preferably located within a micelle of the immobilization material and the compound travels in and out of the immobilization material through transport channels. The micelles of the enzyme immobilization material can be from about 6 nm to about 10 μm, from about 10 nm to 10 μm, from about 10 nm to about 5 μm, from about 10 nm to about 1 μm, from about 10 nm to about 500 nm, from about 10 nm to about 300 nm, from about 10 nm to about 100 nm, from about 10 nm to about 30 nm, from about 15 nm to about 30 nm, from about 20 nm to about 30 nm, from about 25 nm to about 30 nm, from about 6 nm to about 20 nm, or from about 10 nm to about 20 nm. The relative size of the micelles and transport channels can be such that a micelle is large enough to immobilize an enzyme, but the transport channels are too small for the enzyme to travel through them. Further, a transport channel preferably has a diameter of at least about 10 nm.

The micelle diameter to transport channel diameter ratio is at least about 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1 or more; the micelle diameter to transport channel diameter ratio can be about 2:1 to about 10:1, about 2.5:1 to about 10:1, about 3:1 to about 10:1, about 3.5:1 to about 10:1, about 4:1 to about 10:1, about 4.5:1 to about 10:1, about 5:1 to about 10:1, about 5.5:1 to about 10:1, about 6:1 to about 10:1, about 6.5:1 to about 10:1, about 7:1 to about 10:1, about 7.5:1 to about 10:1, about 8:1 to about 10:1, about 8.5:1 to about 10:1, about 9:1 to about 10:1, or about 9.5:1 to about 10:1. Preferably, a transport channel has a diameter of at least about 2 nm and the micelle diameter to transport channel diameter ratio is at least about 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1 or more; the micelle diameter to transport channel diameter ratio can be about 2:1 to about 10:1, about 2.5:1 to about 10:1, about 3:1 to about 10:1, about 3.5:1 to about 10:1, about 4:1 to about 10:1, about 4.5:1 to about 10:1, about 5:1 to about 10:1, about 5.5:1 to about 10:1, about 6:1 to about 10:1, about 6.5:1 to about 10:1, about 7:1 to about 10:1, about 7.5:1 to about 10:1, about 8:1 to about 10:1, about 8.5:1 to about 10:1, about 9:1 to about 10:1, or about 9.5:1 to about 10:1.

When the enzyme is large or aggregated, the enzyme immobilization material can have a micelle size that is substantially the same size as the enzyme or aggregated enzyme. Such an enzyme immobilization material can have micelles that constrain the enzyme or aggregated enzyme in substantially the same space within the enzyme immobilization material and allow diffusion of compounds that are smaller than the enzyme or aggregated enzyme through the material. This enzyme immobilization material would have an average micelle size of from about 15 nm to about 10 μm, from about 15 nm to about 5 μm, from about 15 nm to about 2000 nm, from about 50 nm to about 2000 nm, from about 100 nm to about 2000 nm, from about 200 nm to about 2000 nm, from about 300 nm to about 2000 nm, from about 400 nm to about 2000 nm, from about 500 nm to about 2000 nm, from about 600 nm to about 2000 nm, from about 700 nm to about 2000 nm, from about 800 nm to about 2000 nm, from about 20 nm to about 1000 nm, from about 50 nm to about 1000 nm, from about 100 nm to about 1000 nm, from about 200 nm to about 1000 nm, from about 300 nm to about 1000 nm, from about 400 nm to about 1000 nm, from about 500 nm to about 1000 nm, from about 600 nm to about 1000 nm, or from about 700 nm to about 1000 nm.

Generally, the immobilization material has a micellar or inverted micellar structure. The molecules making up a micelle are amphipathic, meaning they contain a polar, hydrophilic group and a nonpolar, hydrophobic group. The molecules can aggregate to form a micelle, where the polar groups are on the surface of the aggregate and the hydrocarbon, nonpolar groups are sequestered inside the aggregate. In these embodiments, the immobilization material has discrete hydrophobic and hydrophilic regions in the solid state. Inverted micelles have the opposite orientation of polar groups and nonpolar groups. The amphipathic molecules making up the aggregate can be arranged in a variety of ways so long as the polar groups are in proximity to each other and the nonpolar groups are in proximity to each other. Also, the molecules can form a bilayer with the nonpolar groups pointing toward each other and the polar groups pointing away from each other. Alternatively, a bilayer can form wherein the polar groups can point toward each other in the bilayer, while the nonpolar groups point away from each other.

Some of the immobilized enzymes are entrapped in a polymer comprising discrete hydrophilic regions and discrete hydrophobic regions wherein at least one of the hydrophilic regions has a diameter from about 4 nm to about 500 nm and at least one of the other hydrophilic regions has a diameter from about 1 μm to about 300 μm. One of the hydrophilic regions can have a diameter from about 10 nm to about 500 nm. from about 10 nm to about 400 nm, from about 20 nm to about 300 nm, from about 20 nm to about 200 nm and one of the other hydrophilic regions has a diameter from about 1 μm to about 200 μm, from about 1 μm to about 100 μm, from about 1 μm to about 50 μm, from about μm to about 20 μm, from about 1 μm to about 10 μm, from about 1 μm to about 5 μm.

### Modified Polysiloxanes

The micellar or inverted micellar immobilization material can have a structure of Formula 1

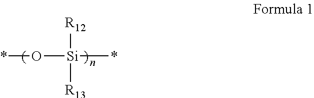

wherein R₁₂ and R₁₃ are hydrogen, alkyl, —O—(SiR₁₄R₁₅—O)ₘ—; or —C₁ to C₁₀ alkyl, —C₁ to C₁₀ alkylene-acid, —C₁ to C₁₀ alkylene-base, or —C₁ to C₁₀ alkylene-enzyme, wherein the —CH₃ group or one or more of the —CH₂— groups can be replaced by an amine group, an oxygen, an amide group, or a carbonyl group or wherein the —CH₃ group or one or more of the —CH₂— groups is substituted with a hydroxy, alkyl, or alkoxy; R₁₄ and R₁₅ are independently alkyl; m, n and q are independently integers from 10 to 1000; provided that the average number of —C₁ to C₁₀ alkylene-enzyme groups per repeat unit is at least 0.03.

Preferably, R₁₂ and R₁₃ are hydrogen, methyl, —O—(Si(CH₃)₂—O—)ₘ—, —C₁ to C₁₀ alkyl, or —C₁ to C₁₀ alkylene-enzyme, wherein the —CH₃ group or one or more of the —CH₂— groups can be replaced by an amine group or an oxygen or wherein the —CH₃ group or one or more of the —CH₂— groups is substituted with a hydroxy or alkyl.

The average number of —C₁ to C₁₀ alkylene-enzyme groups per repeat unit is at least 0.05. 0.1, 0.15, 0.2, 0.25, 0.3, 0.35, or 0.4.

More preferably, R₁₂ and R₁₃ are independently hydrogen, alkyl, —O—(SiR₁₄R₁₅—O)ₘ—; or —C₁ to C₁₀ alkyl, —C₁ to C₁₀ alkylene-acid, —C₁ to C₁₀ alkylene-base, or —C₁ to C₁₀ alkylene-enzyme, wherein the —CH₃ group or one or more of the —CH₂— groups can be replaced by an amine group or an oxygen or wherein the —CH₃ group or one or more of the —CH₂— groups is substituted with a hydroxy or alkyl; R₁₄ and R₁₅ are methyl; and m and n are independently integers from 10 to 1000. An acid group can donate one or more protons and a base can accept one or more protons. In some instances, the acid group can be a carboxylic, a phosphonic, a phosphoric, a sulfonic, a sulfuric, a sulfamate, a salt thereof, or a combination thereof, and the base can be an amine base, particularly, a tertiary amine, a quaternary amine, a nitrogen heterocycle, a salt thereof, or a combination thereof.

Particularly, R₁₂ and R₁₃ are independently hydrogen, alkyl, —O—(Si(CH₃)₂—O)ₘ—, —(CH₂)₃—O—((CH₂)₂₋O)—_{z}CH₃, —(CH₂)₂—C(O)—O—(CH₂)₂-imidazolium, —(CH₂)₃—O—CH₂—CH(OH)—N(CH₃)—(CH₂)₂—SO₃Na, or —(CH₂)₂—C(O)—O—(CH₂)₂-enzyme wherein z is an integer from 2 to 150.

Polysiloxane is a crosslinked polysiloxane reaction product derived from a crosslinking reaction mixture of a grafted polysiloxane and a polysiloxane comprising a vinyl or silanol group wherein the grafted polysiloxane is derived from a grafting reaction mixture comprising a polysiloxane containing silicon hydride (Si—H) bonds and a hydrophilic group having a vinyl or allyl group. The hydrophilic group can be an alkyl group substituted with an acid or a base, a polyether group, or a polyether group substituted with an acid or a base. The grafting reaction mixture can further comprise a catalyst, particularly, a platinum catalyst. The crosslinking reaction mixture can further comprise a catalyst, particularly a metal catalyst.

The structure of Formula 1 is prepared starting with a hydrosiloxane, which is a polysiloxane that contains silicon hydride bonds. Examples include poly(methyl hydrosiloxane) (PMHS) homopolymer, poly(phenyl dimethylhydrosiloxy)siloxane (PPDMHS) homopolymer, and copolymers of PMHS or PPDMHS with other polysiloxanes such as poly(dimethylsiloxane) (PDMS) or poly(phenylmethylsiloxane) (PPMS). Specifically, further examples include polyalkyl hydrosiloxane (e.g., poly(methyl hydrosiloxane), poly(ethyl hydrosiloxane), poly(propyl hydrosiloxane), polyaryl hydrosiloxane (e.g., poly(phenyl hydrosiloxane), poly(tolyl hydrosiloxane)), poly(phenyl dimethylhydrosiloxy)siloxane, poly(dimethyl siloxane co-methyl hydrosiloxane), poly(methyl hydrosiloxane co-phenyl methyl siloxane), poly(methyl hydrosiloxane co-alkyl methyl siloxane), poly(methyl hydrosiloxane co-diphenyl siloxane), poly(methyl hydrosiloxane co-phenyl methyl siloxane). These polysiloxanes have a desirable CO₂ solubility. Without being bound by theory, it is believed that the elasticity of polysiloxanes increases CO₂ solubility.

Using published procedures, these hydride-functional polysiloxanes can be grafted with polyether and/or ionic groups by coupling them with allyl-containing compounds using a platinum catalyst (hydrosilation reaction). The general reaction schemes are shown in Schemes 2-7. In the general synthesis schemes below, the R groups can be alkyl.

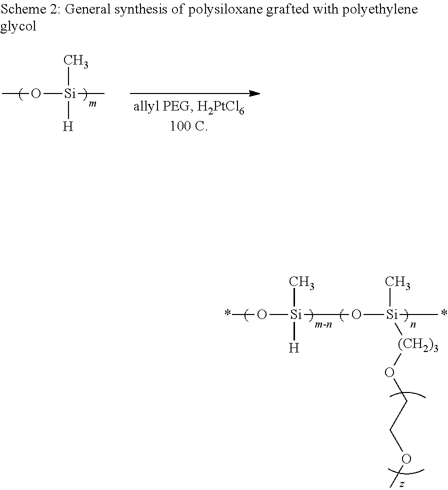

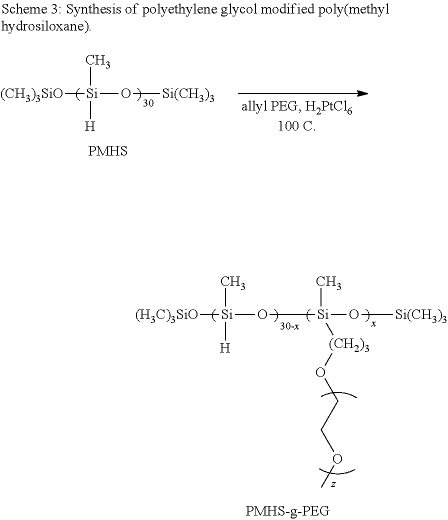

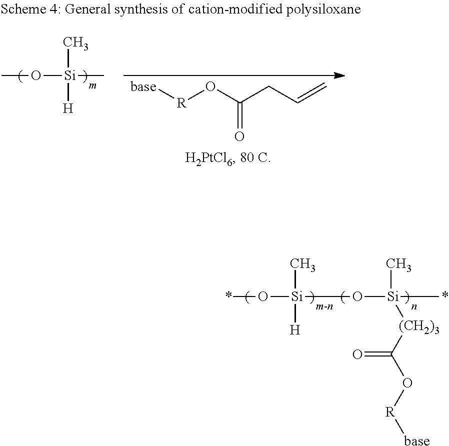

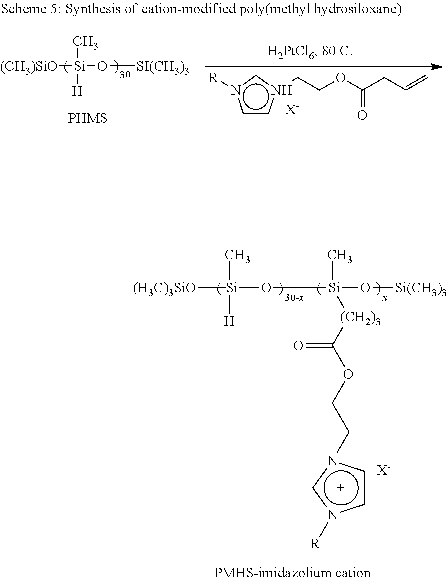

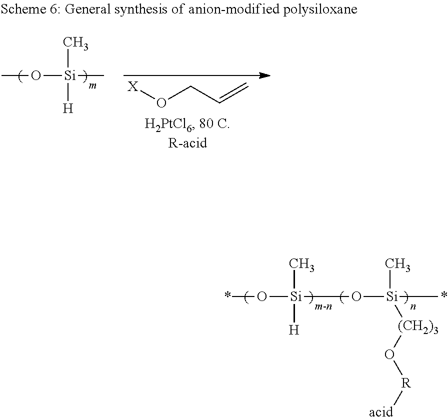

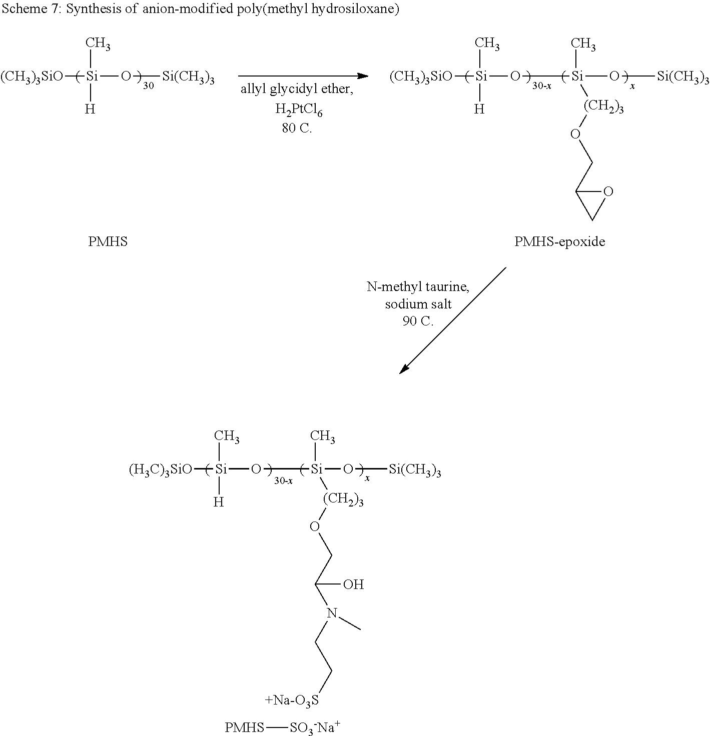

Generally, functionalization of an ionic and nonionic polysiloxane can be manipulated by controlling the amount of polyether or ionic groups added. In particular, functionalization of PMHS can be varied by varying the amount of allyl PEG, allyl glycidyl ether, and/or alkylimidazolium acrylate added to the reaction mixture. Addition of functional sites (e.g., polyether or ionic groups) increases the water solubility of ionic and nonionic polysiloxanes. The water solubility of the polymer depends on the number of functional sites added to the polysiloxane. Further, polysiloxanes can be functionalized with both a polyether and an ionic species by adding a polyether having an allyl group and an ionic compound having an allyl group to the same reaction mixture.

The functionalized PMHS can then be crosslinked into an elastomer having properties similar to a natural rubber by using the remaining Si—H groups via two possible pathways, a hydrosilylation reaction or a dehydrogenative coupling reaction. The hydrosilylation reaction uses a platinum catalyst such as platinum-divinyltetramethyldisiloxane complex and vinyl-functional polysiloxanes as crosslinkers. Examples of vinyl-functional polysiloxanes include divinyl-terminated PDMS or PPMS, poly(vinylmethylsiloxane) (PVMS) homopolymer, and copolymers of PVMS and PDMS or PPMS. The dehydrogenative coupling reaction uses a catalyst wherein the choice of catalyst depends on the coupling mechanism. Tin catalysts are predominately used in dehydrogenative coupling reaction where Si—H couples to Si—OH to form —Si—O—Si— linkages. Tin catalysts such as di-n-butyldilauryltin are used with silanol-functional polysiloxanes as crosslinkers. In addition to tin compounds, other transition metal complexes based on zinc, iron, cobalt, ruthenium, iron, rhodium, iridium, palladium, and platinum can be used. Specific examples include zinc octoate, iron octoate, and Wilkinson's catalyst (rhodium-based metal salt; (PhP)₃RhCl). Precious metal catalysts (predominately platinum but rhodium as well) are used in hydrosilylation reactions where Si—H reacts with a terminal vinyl bond to form —S₁—CH₂—CH₂—Si—. Free radical initiators (thermal and/or UV generated) can be used to crosslink vinyl, acrylate, or methacrylate containing polysiloxanes. Tin and/or titanium compounds are used to catalyze condensation cure systems where Si—OH groups react with a variety of reactive groups (alkoxy, acetoxy, oxime, epoxy, and amines) to form —Si—O—Si— bonds. These condensation cure systems are moisture sensitive and will react in the presence of water only, but using titanium and/or tin compounds speeds up that reaction. Examples of silanol-functional polysiloxanes include disilanol-terminated PDMS or poly(trifluoropropylmethylsiloxane) (PTFPMS), disilanol-terminated copolymers of PPMS and PDMS, and silanol-trimethylsilyl modified Q resins. The crosslink density affects the material's properties and enzyme retention in the immobilization matrix.

Other variables to this immobilization procedure include annealing temperature (4° C.-60° C. for bovine carbonic anhydrase (BCA) or to 80° C. for human carbonic anhydrase (HCA)) and tin catalyst choice and loading. In addition, to dibutyldilauryltin, bis(2-ethylhexanoate)tin, dimethylhydroxy(oleate)tin, and dioctyldilauryltin can be used as the catalyst. As the annealing temperature increases, the amount of tin catalyst needed to maintain a desirable reaction rate (solidifying in 30 minutes or less) decreases and ranges from about 0.01 to about 10 vol. %, preferably about 0.2 to about 4 vol. %.

Additionally, PMHS-g-PEG can be crosslinked via a different mechanism (hydrosilylation) using precious metal catalysts and vinyl-containing polysiloxane crosslinkers of various molecular weights. Useful catalysts for this reaction are platinum-divinyltetramethyldisiloxane complex, platinum-cyclovinylmethylsiloxane complex, and tris(dibutylsulfide)rhodium trichloride at loadings of about 0.01 to about 5 vol. %, preferably about 0.02 to about 0.5 vol. %. Examples of vinyl-containing polysiloxane crosslinkers are divinyl terminated poly(dimethylsiloxane), divinyl terminated diphenylsiloxane-dimethylsiloxane copolymer, divinyl terminated poly(phenylmethylsiloxane), poly(vinylmethylsiloxane), vinyl Q resins, vinyl T structure polymers, vinylmethylsiloxane-dimethylsiloxane copolymer, and poly(vinylphenylsiloxane co-phenylmethylsiloxane).

PHMS-g-PEG micellar polymers can be crosslinked via their remaining reactive silicon hydride (Si—H) groups into a rubbery solid via one of two mechanisms, a hydrosilylation reaction or a dehydrogenative coupling reaction. The hydrosilylation reaction uses a platinum catalyst such as platinum-divinyltetramethyldisiloxane complex and vinyl-functional polysiloxanes as crosslinkers. Examples of vinyl-functional polysiloxanes include divinyl-terminated PDMS or PPMS, poly(vinylmethylsiloxane) (PVMS) homopolymer, and copolymers of PVMS and PDMS or PPMS. The dehydrogenative coupling reaction uses a tin catalyst such as di-n-butyldilauryltin and silanol-functional polysiloxanes as crosslinkers. Examples of silanol-functional polysiloxanes include disilanol-terminated PDMS or poly(trifluoropropylmethylsiloxane) (PTFPMS), disilanol-terminated copolymers of PPMS and PDMS, and silanol-trimethylsilyl modified Q resins.

Speier's catalyst is easily poisoned by the presence of a variety of functional groups (e.g., amines, carboxylates, cyano groups, epoxides, etc.) during the hydrosilylation reaction (Sabourault, N.; Mignani, G.; Wagner, A.; Mioskowski, C. Organic Letters 2002, 4(13), 2117-2119). However, some heterogeneous catalysts, such as platinum oxide or platinum on carbon (Chauhan, M.; Hauck, B. J.; Keller, L. P.; Boudjouk, P. Journal of Organometallic Chemistry 2002, 645, 1-13), have been used to graft these functional groups onto a silicon hydride containing compound at elevated temperatures.

The second class of platinum catalyst that can be used is one that is active at room temperature (i.e., a “hot” catalyst), particularly, Karstedt's catalyst (platinum-divinyltetramethyldisiloxane complex) (Chung, D.-W.; Kim, T. G. Journal of Industrial Engineering and Chemistry 2007, 13(4), 571-577). This catalyst is known to be more compatible with various functional groups such as amines and carboxylates. Because this catalyst is active at room temperature, the catalyst must be removed or otherwise deactivated to prevent the resulting graft copolymers from crosslinking through the unreacted silicon hydride groups. Several methods for removing this catalyst exist, including catalyst deactivation after reaction using triphenylphosphine, precipitation of the graft copolymer in hexanes, or binding the catalyst to an inorganic substrate such as Magnasol.

Several functional groups can be attached to the end of polyethylene glycol (PEG) chains and grafted into polysiloxane micellar or inverted micellar polymers. These functional groups are capable of reacting with the amino, carboxyl, hydroxyl, or sulfhydryl groups on the surface of an enzyme, particularly carbonic anhydrase (CA), to covalently tether the enzyme into the hydrophilic regions of the micellar or inverted micellar polymer.

The considerations for an appropriate system for immobilizing enzymes include compatibility of the PEG derivative with the rest of the chemistry tethering the polymer to the enzyme. Desirably, the functionalized PEGs would not react under the reaction conditions (100° C., toluene, 24 hours; or room temperature in toluene with a hot catalyst), would not poison the platinum catalyst, and would not catalyze crosslinking during contact with the reagents. The reaction of the derivatized PEGs can be tested before incorporating the enzyme into the immobilization material.

### Modified Polysulfone

In some of the various embodiments, the immobilization material has a structure of Formula 7

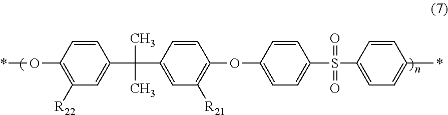

wherein R₂₁ and R₂₂ are independently hydrogen, alkyl, or alkylene-enzyme wherein one or more of the —CH₂— groups can be replaced by an amine group, an oxygen, an amide group, or a carbonyl group or wherein one or more of the —CH₂— groups is substituted with a hydroxy, alkyl, or alkoxy; n is an integer of 10 or greater, provided that the average number of alkyl or alkylene-enzyme groups per repeat unit is at least 0.03. In various embodiments, R₂₁ and R₂₂ are independently hydrogen, alkyl, or alkylene-enzyme. In various embodiments, R₂₁ and R₂₂ are independently hydrogen, alkylene-enzyme, or —(CH₂)_{q}N⁺R₂₆R₂₇R₂₈, wherein R₂₆, R₂₇, and R₂₈ are independently alkyl and q is an integer of 1, 2, or 3; particularly, R₂₆, R₂₇, and R₂₈ are independently methyl, ethyl, propyl, butyl, pentyl, or hexyl; more particularly, R₂₆, R₂₇, and R₂₈ are methyl.

Alternatively, R₂₁ and R₂₂ are independently hydrogen, alkylene-enzyme, or —(CH₂)_{q}N⁺R₂₆R₂₇R₂₈, wherein R₂₆ and R₂₇ are independently methyl, ethyl, or propyl, R₂₈ is alkylamino, and q is an integer of 1, 2, or 3. When R₂₈ is alkylamino, preferred alkylamino groups are tertiary alkylamino groups. For example, the alkylamino group can be —CH₂N⁺R₂₉R₃₀R₃₁, —CH₂CH₂N⁺R₂₉R₃₀R₃₁ or —CH₂CH₂CH₂N⁺R₂₉R₃₀R₃₁ wherein R₂₉, R₃₀, and R₃₁ are independently hydrogen or alkyl. In various preferred embodiments, R₂₉, R₃₀, and R₃₁ are independently methyl, ethyl, propyl, butyl, pentyl, or hexyl; more particularly, R₂₉, R₃₀, and R₃₁ are methyl or ethyl.

Preferably, R₂₁, R₂₂, or R₂₁ and R₂₂ are alkyl or alkylene-enzyme wherein the average number of alkyl or alkylene-enzyme groups per repeat unit is from about 0.03 to about 1.4, from about 0.05 to about 1.4, from about 0.1 to about 1.4, from about 0.2 to about 1.2, from about 0.2 to about 1, from about 0.3 to about 0.8, from about 0.4 to about 1.4, from about 0.4 to about 1.2, from about 0.4 to about 1, from about 0.4 to about 0.8, from about 0.5 to about 1.4, from about 0.5 to about 1.2, from about 0.5 to about 1, from about 0.5 to about 0.8.

In other preferred embodiments, R₂₁ and R₂₂ are independently hydrogen, alkylene-enzyme wherein one or more of the —CH₂— groups can be replaced by an amine group, an oxygen, an amide group, or a carbonyl group or wherein one or more of the —CH₂— groups is substituted with a hydroxy, alkyl, or alkoxy, or —(CH₂)_{q}-polyether wherein q is an integer of 1, 2, or 3. In preferred embodiments, q is 1. In some of the preferred embodiments, R₂₁ and R₂₂ are independently hydrogen, —CH₂—O—(CH₂(CH₃)—CH₂—O)_{z}—Rₜ, —CH₂—O—(CH₂—CH₂—O)_{z}—Rₜ, or a combination thereof wherein z is an integer from 3 to 180, and the polyethylene oxide or polypropylene oxide (e.g., —O—(CH₂—CH₂—O)_{z}—Rₜ or —CH₂—O—(CH₂(CH₃)—CH₂—O)_{z}—Rₜ wherein Rₜ is hydrogen, alkyl, alkylene-enzyme, or aryl) has a molecular weight from about 150 Daltons (Da) to about 8000 Daltons (Da). In particular embodiments, the polyethylene oxide has a molecular weight from about 500 Da to about 600 Da; particularly about 550 Da.

Modified polysulfone is a desirable immobilization material because it has good chemical and thermal stability. Additionally, modified polysulfone has advantageous solubility characteristics in polar organic solvents such as N-methylpyrrolidone (NMP) and dioxane. This solubility enables the modified polysulfone beads to be prepared by precipitation in water or lower aliphatic alcohols. Unmodified polysulfone can immobilize and retain an enzyme (e.g., carbonic anhydrase) in the beads. But, the activity of the carbonic anhydrase is reduced and it is hypothesized that the low porosity and thus, the low permeability of unmodified polysulfone beads at the polymer-solvent interface prevents the substrate and product from diffusing to and from the active site of the enzyme. In order to improve the porosity, the polysulfone can be modified to increase the porosity and transport of the substrate and product through the material.

For example, the polysulfone can be modified by adding amine groups to the benzene groups of the polysulfone. By modifying the polysulfone with quaternary amine groups, the hydrophilicity of the polysulfone is affected and in turn the porosity and the transport of carbonate/bicarbonate ions increases. Also, the positively charged amine groups can stabilize carbonic anhydrase through electrostatic interactions. This modification of adding a hydrophobic group to a hydrophilic polymer may also form micellar aggregate/pore structures in the polymer. To add amine groups to the polysulfone, the benzene rings of the backbone are chloromethylated followed by the amination of the chloromethyl groups. This process is generally described in Jihua, H.; Wentong, W.; Puchen, Y.; Qingshuang, Z. Desalination 1991, 83, 361 and Park, J.-S.; Park, G.-G.; Park, S.-H.; Yoon, Y.-G.; Kim, C. S.; Lee, W. Y. Macromol. Symp. 2007, 249-250, 174. The general reaction scheme for this transformation is shown in Scheme 8. The average number of chloromethyl groups added per repeat unit can be controlled by manipulating the reactant ratios during the first step as described in Hibbs, M. R.; Hickner, M. A.; Alam, T. M.; McIntyre, S. K.; Fujimoto, C. H.; Cornelius, C. J. Chem. Mater. 2008, 20, 2566.

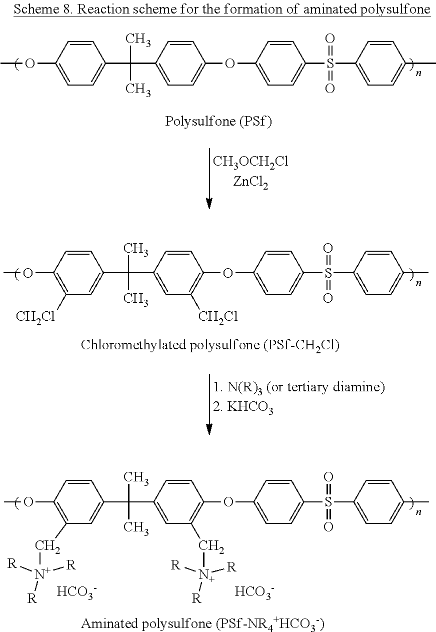

Additionally, the choice of tertiary amine added to the chloromethylated polysulfone (PSf-CH₂Cl) can affect the polysulfone properties. For instance, trimethyl amine can be used to aminate PSf-CH₂Cl, resulting in a quaternary benzyl trimethyl ammonium cation. This benzyl trimethyl ammonium cation has been shown to be more stable with prolonged exposure to elevated temperatures and/or strongly basic solutions. (See Sata, T.; Tsujimoto, M.; Yamaguchi, T.; Matsusaki, K. J. Membrane Sci. 1996, 112, 161.) Tertiary diamines can also be used in this amination step, providing a way of crosslinking polysulfone to improve its mechanical and thermal stability. The addition of diamines to chloromethylated polysulfone solutions crosslinks polysulfone and solidifies the mixture. The solvent can then be exchanged with water or methanol to yield a more porous aminated polysulfone. The initial polymer concentration of the solution can be adjusted to manipulate the porosity in the resulting polysulfone. The exchange of the chloride anions with bicarbonate anions after amination could improve the performance of the immobilized carbonic anhydrase by removing chloride ions that inhibit enzyme activity. Additionally, the incorporation of bicarbonate ions into polysulfone could provide a buffering capacity to protect the enzyme from pH changes.

Further, once the polysulfone is chloromethylated, other modified polysulfone polymers can be prepared. For example, the chloromethyl groups can react with a hydroxyl end group of poly(ethylene oxide) (PEO) to create polysulfone polymers with grafted PEO side chains. (See Park, J. Y.; Acar, M. H.; Akthakul, A.; Kuhlman, W.; Mayes, A. M. Biomater. 2006, 27, 856.) The general reaction scheme is shown in Scheme 9. As described above, the chloromethylation of polysulfone can be manipulated to provide control over the grafting density of the PEO side chains. Additionally, the molecular weight of the PEO side chains can be altered to influence the overall weight loading of PEO in PEO-modified polysulfone; the loading affects the overall mechanical properties of the polymer.

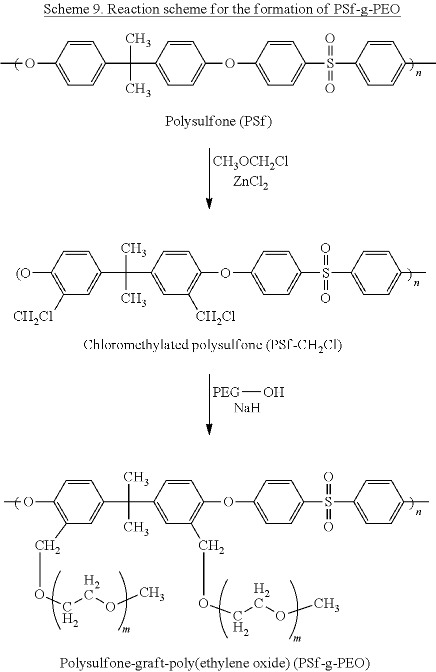

The incorporation of PEO into polysulfone will improve the hydrophilicity of these beads and the transport of carbonate/bicarbonate ions. Additionally, when polyethylene glycol-modified carbonic anhydrase is the enzyme, the PEO-modified polysulfone can provide a hydrophilic PEO layer around the carbonic anhydrase and further prevent the enzyme from leaching. The PEO encapsulation of carbonic anhydrase can also protect the enzyme from effects of drying that may be important for retaining its activity upon immobilization.

Additionally, particular processing conditions can also improve the porosity and the ion transport of the polymers. For instance, it is possible to foam polysulfone through the use of supercritical carbon dioxide to introduce microporous structure into polysulfone polymers. (See Krause, B.; Mettinkhof, R.; van der Vegt, N. F. A.; Wessling, M. Macromolecules 2001, 34, 874.) A similar approach could be used to enable the foaming of modified polysulfone beads. Microporosity can also be introduced into polysulfone by using a freeze-drying process similar to the process used to create microporous chitosan. (See Cooney, M. J.; Lau, C.; Windmeisser, M.; Liaw, B. Y.; Klotzbach, T.; Minteer, S. D. J. Mater. Chem. 2008, 18, 667.) Since polysulfone is not soluble in a water/acetic acid mixture, a suitable solvent for polysulfone that is capable of appreciable sublimation in its solid state under vacuum is required. Menthol is a promising candidate due to its low melting temperature (35° C.) and comparable solubility parameter to dioxane, which suggests that polysulfone could dissolve at high concentrations in menthol at slightly elevated temperatures.

In these embodiments, polysulfone is a reaction product derived from a functionalization reaction mixture comprising a product of a chloromethylation reaction mixture and a functionalizing agent (e.g., amine or polyether) wherein the functionalizing agent comprises a functional group that can react with an enzyme as described below. The chloromethylation reaction mixture comprises polysulfone and a chloromethylation reagent.

### Modified Polycarbonate

In certain embodiments, the immobilization material has a structure of Formula 8

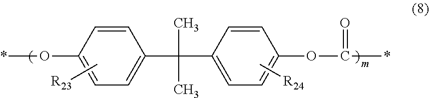

wherein R₂₃ and R₂₄ are independently hydrogen, alkyl, or alkylene-enzyme wherein one or more of the —CH₂— groups can be replaced by an amine group, an oxygen, an amide group, or a carbonyl group or wherein one or more of the —CH₂— groups is substituted with a hydroxy, alkyl, or alkoxy, provided that the average number of alkyl or alkylene-enzyme groups per repeat unit is at least 0.03. In various embodiments, R₂₃ and R₂₄ are independently hydrogen, alkyl, or alkylene-enzyme. In various embodiments, R₂₃ and R₂₄ are independently hydrogen, alkylene-enzyme or —(CH₂)_{q}N⁺R₂₆R₂₇R₂₈, wherein R₂₆, R₂₇, and R₂₈ are independently alkyl and q is an integer of 1, 2, or 3; particularly, R₂₆, R₂₇, and R₂₈ are independently methyl, ethyl, propyl, butyl, pentyl, or hexyl; more particularly, R₂₆, R₂₇, and R₂₈ are methyl.

Alternatively, R₂₃ and R₂₄ are independently hydrogen, alkylene-enzyme or —(CH₂)ₚN⁺R₂₆R₂₇R₂₈ wherein R₂₆ and R₂₇ are independently methyl, ethyl, or propyl, R₈ is alkylamino, and p is an integer of 1, 2, or 3. When R₂₈ is alkylamino, preferred alkylamino groups are tertiary alkylamino groups. For example, the alkylamino group can be —CH₂N⁺R₂₉R₃₀R₃₁, —CH₂CH₂N⁺R₂₉R₃₀R₃₁ or —CH₂CH₂CH₂N⁺R₂₉R₃₀R₃₁ wherein R₂₉, R₃₀, and R₃₁ are independently hydrogen or alkyl. In various preferred embodiments, R₂₉, R₃₀, and R₃₁ are independently methyl, ethyl, propyl, butyl, pentyl, or hexyl; more particularly, R₂₉, R₃₀, and R₃₁ are methyl or ethyl.

In other preferred embodiments, R₂₃ and R₂₄ are independently hydrogen, alkylene-enzyme or —(CH₂)_{q}-polyether wherein q is an integer of 1, 2, or 3. In some of the preferred embodiments, R₂₃ and R₂₄ are independently hydrogen, —CH₂—O—(CH₂(CH₃)—CH₂—O)_{z}—Rₜ, —CH₂—O—(CH₂—CH₂—O)_{z}—Rₜ, or a combination thereof wherein z is an integer from 3 to 180, and the polyethylene oxide or polypropylene oxide (e.g., —O—(CH₂—CH₂—O)_{z}—Rₜ or —CH₂—O—(CH₂(CH₃)—CH₂—O)_{z}—Rₜ wherein Rₜ is hydrogen, alkyl, alkylene-enzyme, or aryl) has a molecular weight from about 150 Daltons (Da) to about 8000 Daltons (Da).

Preferably, R₂₃, R₂₄, or R₂₃ and R₂₄ are alkyl or alkylene-enzyme wherein the average number of alkyl or alkylene-enzyme groups per repeat unit is from about 0.03 to about 1.4, from about 0.05 to about 1.4, from about 0.1 to about 1.4, from about 0.2 to about 1.2, from about 0.3 to about 1, from about 0.3 to about 0.8, from about 0.4 to about 1.4, from about 0.4 to about 1.2, from about 0.4 to about 1, from about 0.4 to about 0.8, from about 0.5 to about 1.4, from about 0.5 to about 1.2, from about 0.5 to about 1, from about 0.5 to about 0.8.

Polycarbonate has a structure similar to polysulfone. It also contains benzene rings in its backbone, so it can be functionalized by adding chloromethyl groups in the same manner as described above for polysulfone. These chloromethyl groups can then be aminated or have PEO grafted following the same procedure utilized for polysulfone. Schemes 10 and 11 show the general reaction schemes for both. Similar to polysulfone, polycarbonate can be foamed using supercritical carbon dioxide.

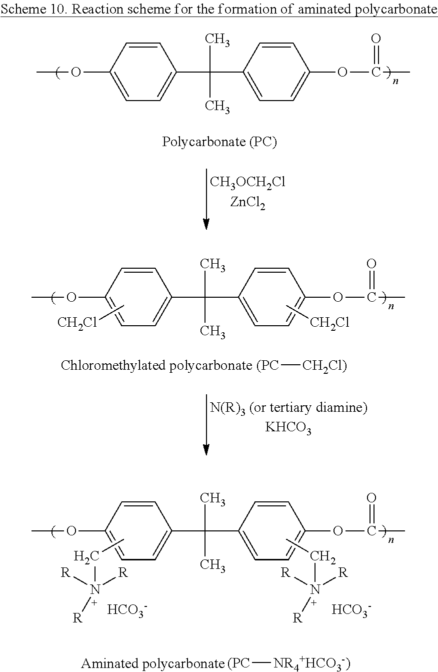

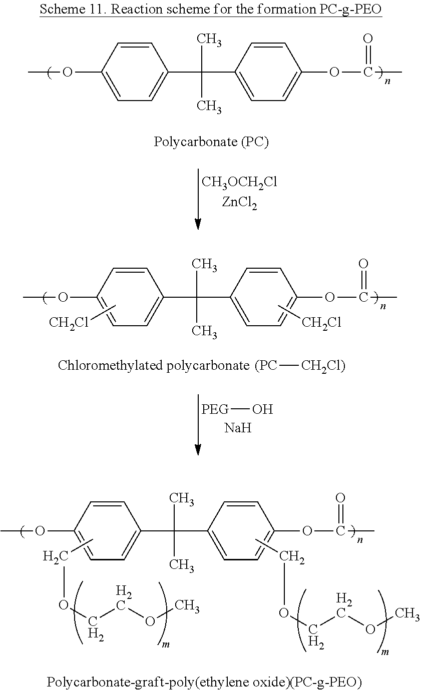

In these embodiments, polycarbonate is a reaction product derived from a functionalization reaction mixture comprising a product of a chloromethylation reaction mixture and a functionalizing agent (e.g., amine or polyether) wherein the functionalizing agent comprises a functional group that can react with an enzyme as described below. The chloromethylation reaction mixture comprises polycarbonate and a chloromethylation reagent.

### Modified Poly(Vinylbenzyl Chloride)

In other embodiments, the immobilization material has a structure of Formula 9

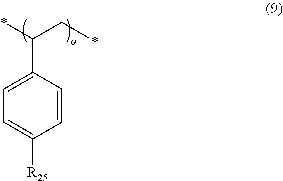

wherein R₂₅ is hydrogen, alkyl, or alkylene-enzyme wherein one or more of the —CH₂— groups can be replaced by an amine group, an oxygen, an amide group, or a carbonyl group or wherein one or more of the —CH₂— groups is substituted with a hydroxy, alkyl, or alkoxy, o is an integer of 10 or greater; provided that the average number of alkyl or alkylene-enzyme groups per repeat unit is at least 0.03. In various embodiments, R₂₅ is hydrogen, alkyl, or alkylene-enzyme. In various embodiments, R₂₅ is hydrogen, alkylene-enzyme, or —(CH₂)_{q}N⁺R₂₆R₂₇R₂₈, wherein R₂₆, R₂₇, and R₂₈ are independently alkyl and q is an integer of 1, 2, or 3; particularly, R₂₆, R₂₇, and R₂₈ are independently methyl, ethyl, propyl, butyl, pentyl, or hexyl; more particularly, R₂₆, R₂₇, and R₂₈ are methyl.

Alternatively, R₂₅ is hydrogen, alkylene-enzyme or —(CH₂)ₚN⁺R₂₆R₂₇R₂₈ wherein R₂₆ and R₂₇ are independently methyl, ethyl, or propyl, R₂₈ is alkylamino, and p is an integer of 1, 2, or 3. When R₂₈ is alkylamino, preferred alkylamino groups are tertiary alkylamino groups. For example, preferred alkylamino groups can be —CH₂N⁺R₂₉R₃₀R₃₁, —CH₂CH₂N⁺R₂₉R₃₀R₃₁ or —C₆H₄N⁺R₂₉R₃₀R₃₁ wherein R₂₉, R₃₀, and R₃₁ are independently hydrogen or alkyl. In various preferred embodiments, R₂₉, R₃₀, and R₃₁ are independently methyl, ethyl, propyl, butyl, pentyl, or hexyl; more particularly, R₂₉, R₃₀, and R₃₁ are methyl or ethyl.

Preferably, R₂₅ is alkyl or alkylene-enzyme wherein the average number of alkyl or alkylene-enzyme groups per repeat group is at least 0.03, 0.05, 0.1, 0.2, 0.3, 0.4, 0.5, 0.6, 0.7, or more.

Poly(vinylbenzyl chloride) (PVBC) is a commercially-available polymer with a chloromethyl group contained in the polymer, so it can be aminated similarly to the synthetic procedure described above for chloromethylated polysulfone or polycarbonate. PVBC, however, lacks the mechanical strength of polysulfone and polycarbonate and is somewhat brittle and has a lower glass transition temperature. However, it is believed that the mechanical and thermal stability of this polymer can be improved by crosslinking PVBC by amination with tertiary diamines. (See Varcoe, J. R.; Slade, R. C. T.; Lee, E. L. H. Chem. Commun. 2006, 1428.) This process incorporates positive charges in the PVBC and these charges can also stabilize the immobilized enzyme through electrostatic interactions. Scheme 12 shows the general scheme for this reaction.

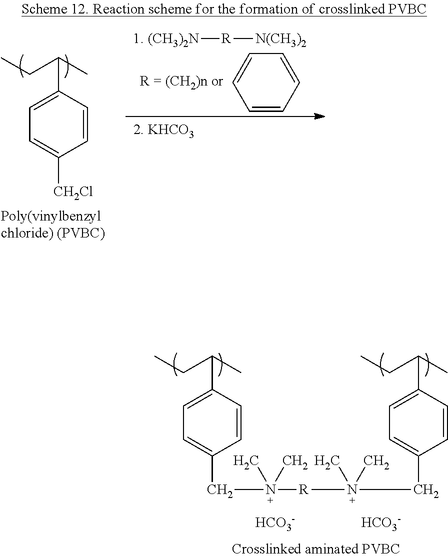

Upon addition of a diamine to a 40 wt. % solution of PVBC in NMP, both a methylene (—CH₂—) and a phenylene (—C₆H₄—) spacer in the diamine produces crosslinked solid films. Diamines having the following structures were selected because they provide long-term stability to these quaternary amines. The use of tetramethyl methanediamine (TMMDA) solidifies this solution quickly (e.g., less than 10 minutes), indicating that the reaction of TMMDA with PVBC is fast. Once solidified, PVBC crosslinked with TMMDA does not swell upon addition of methanol or water. In contrast, the reaction of tetramethyl phenylenediamine (TMPDA) is slower and takes several hours to solidify. Once solidified, PVBC crosslinked with TMPDA swells significantly (but maintains its original shape) upon exposure to either methanol or water. PVBC crosslinked with TMPDA forms a hydrophilic, high-swelling material, which could significantly improve the transport of carbonate/bicarbonate ions through the polymer, as compared to polysulfone and polycarbonate that are rigid glassy polymers. Similar to the polysulfone and polycarbonate, the amount of derivatization of the modified PVBC can be altered by adjusting the polymer concentration of the solution during the chloromethylation reaction.

In these embodiments, poly(vinylbenzyl chloride) is a reaction product derived from a crosslinking mixture comprising a product of a functionalization reaction mixture and a crosslinking agent (e.g., diamine). The functionalization reaction mixture comprises poly(vinylbenzyl chloride) and a functionalizing agent wherein the functionalizing agent comprises a functional group that can react with an enzyme as described below.

The enzyme aggregates described herein can be immobilized in the polymeric micellar or inverted micellar material described herein. In some of the embodiments, the enzyme aggregates can be immobilized in a non-ionic polymeric micellar or inverted micellar immobilization material. The non-ionic polymeric immobilization materials are described herein where the polymers do not have acid or base groups attached to pendant hydrophilic moieties. Further the non-ionic polymeric immobilization materials can be polysiloxane, polysulfone, polycarbonate, or poly(vinylbenzyl chloride) based polymers.

Further, for example, the enzyme aggregates from crosslinking or from intermolecular interactions can be immobilized in a polymer having the structure of Formula 6:

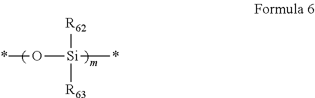

wherein R₆₂ and R₆₃ are hydrogen, alkyl, —O—(SiR₆₄R₆₅—O)ₘ—; or —C₁ to C₁₀ alkyl, —C₁ to C₁₀ alkylene-acid, —C₁ to C₁₀ alkylene-base, wherein the —CH₃ group or one or more of the —CH₂— groups can be replaced by an amine group, an oxygen, an amide group, or a carbonyl group or wherein the —CH₃ group or one or more of the —CH₂— groups is substituted with a hydroxy, alkyl, or alkoxy; R₆₄ and R₆₅ are independently alkyl; and m and n are independently integers from 10 to 1000. In various preferred embodiments, R₆₄ and R₆₅ are methyl.

### Ionic Conductivity

Certain alkali metal salts complex with polyethers such as PEG to improve the polyether ionic conductivity. In fact, recent work has shown that the addition of lithium bis(trifluoromethylsulfonyl)imide prior to crosslinking can improve the ionic conductivities of PHMS-g-PEG by several orders of magnitude. (Zhang, Z. C.; Jin, J. J.; Bautista, F.; Lyons, L. J.; Shariatzadeh, N.; Sherlock, D.; Amine, K.; West, R. Solid State Ionics 2004, 170, 233-238; Zhang, Z.; Sherlock, D.; West, R.; West, R. Macromolecules 2003, 36, 9176-9180) The molar ratio of ethylene glycol repeat units to alkali cation as well as the type of alkali cation used are factors that affect the ionic conductivity of these systems.

Either anionic or cationic groups can be grafted to the PEG regions of the polysiloxane micellar or inverted micellar polymers.

Polysiloxanes can be grafted with reactive epoxide groups. The epoxide groups are ring-opened with sulfonate-containing compounds to introduce anionic sulfonate groups onto the PHMS backbone. Epoxide groups can also be reacted with carboxylic acid-containing compounds to introduce anionic carboxylate groups onto PHMS, shown in Scheme 13 (Bauer, J.; Husing, N.; Kickelbick, G. Journal of Polymer Science Part A: Polymer Chemistry 2004, 42, 3975-3985). Other reagents could be used to incorporate other anionic species such as phosphate and nitrate groups onto PHMS.

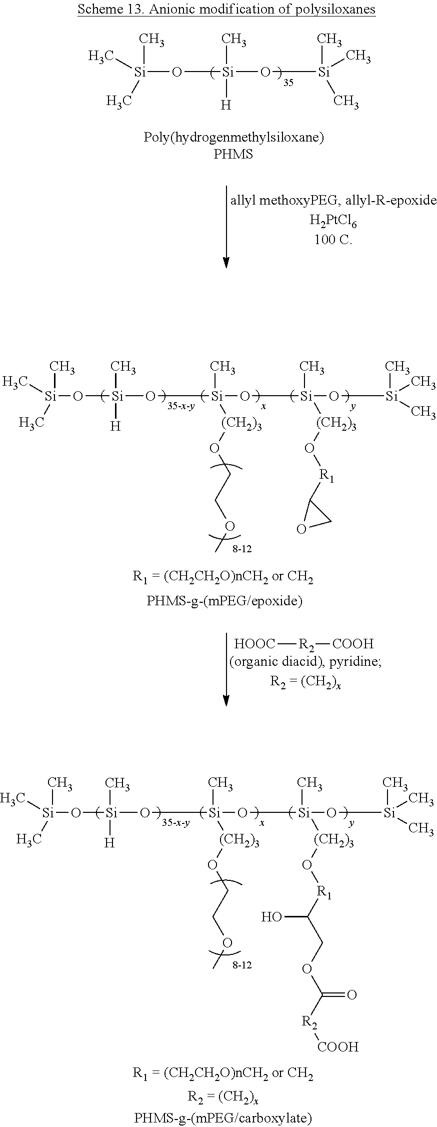

Cationic species such as imidazolium ions can be grafted directly onto PHMS. Additionally, an epoxide-functionalized PHMS can react with various amine-containing compounds such as 3,5-dimethylaniline, hexylamine, or diethylamine to introduce primary or secondary amines onto PHMS, as shown in the scheme below. (Bauer, J.; Husing, N.; Kickelbick, G. Journal of Polymer Science Part A: Polymer Chemistry 2004, 42, 3975-3985; Senthilkumar, U.; Reddy, B. S. R. Journal of Membrane Science 2004, 232, 73-83) These amines can then be converted into cationic species upon exposure to acids or alkylating agents such as methyl iodide. Tertiary amines such as N,N-dimethylallylamine can be directly grafted onto the PHMS backbone, as shown in the second scheme. (Kang, J. J.; Li, W. Y.; Lin, Y.; Li, X. P.; Xiao, X. R.; Fang, S. B. Polymers for Advanced Technologies 2004, 15, 61-64) These tertiary amines can then be converted into quaternary ammonium groups via exposure to acids or to alkylating agents such as methyl iodide.

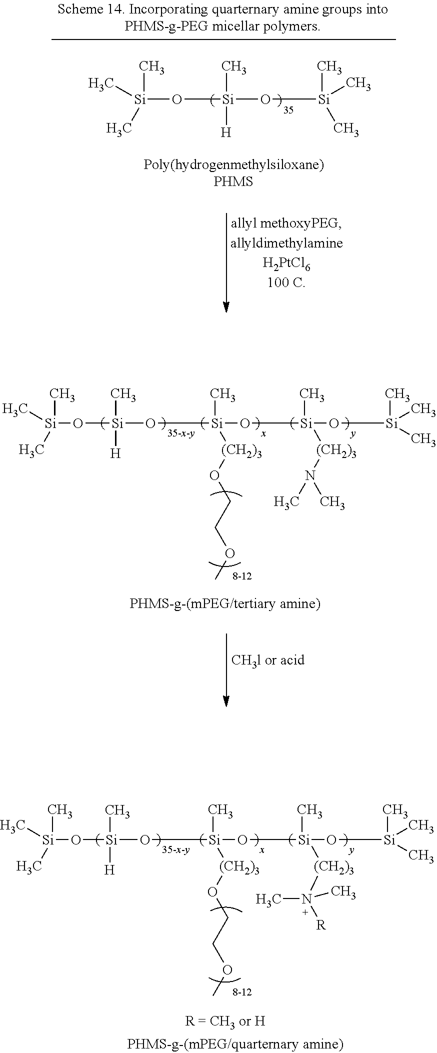

There is a crosslinking reaction that eliminates the need for the addition of the hydrophobic disilanol-terminated PDMS to crosslink PHMS-g-PEG. This reaction grafts the silicon-hydride groups of PHMS with carboxylate groups or PEG chains. The resulting micellar polymers can then be crosslinked by reaction with various multifunctional amines (e.g., tris(2-aminoethyl)amine, ethylenediamine, 1,3-diaminopropane, diethylene triamine, triethylamine tetramine, 1,4-diaminobutane, and the like), polyamines (e.g., polyethyleneimine (PEI), poly(allyl amine), poly(lysine), polyvinyl amine, polyguanidine, poly(arginine), and the like) and adding a carbodiimide such as 1-ethyl-3-[3-dimethylaminopropyl]carbodiimide hydrochloride (EDC) as a catalyst to react the carboxylate groups with the amine groups. Because these amine-containing compounds are also hydrophilic, the resulting crosslinked polymer would be significantly more hydrophilic than one that uses disilanol-terminated PDMS as a crosslinker. Further, since not all of the carboxylates and amines will react during the crosslinking reaction, excess ionic groups will remain to improve ionic conductivity of the polymer.

Additionally, this reaction provides for tethering of CA inside the micellar or inverted micellar polymer. The enzyme can be added to the micellar polymer/polyamine mixture before EDC addition. Upon EDC catalyst addition, carboxylates and/or amines on the enzyme surface will react to provide covalent attachment of the enzyme to the hydrophilic regions of the micellar polymer.

PHMS-g-PEG can be crosslinked through zwitterionic carboxylate groups attached to the PHMS backbone and does not require hydrophobic PDMS as a crosslinker. While this crosslinking reaction is similar to the reaction above, it provides a facile way to covalently bond the enzyme to the micellar or inverted micellar polymer mixture during EDC-catalyzed crosslinking. Also, this reaction provides more flexibility in the type and number of ionic groups incorporated into the crosslinked polymer. In this reaction, a bromine- or chlorine-containing group is grafted onto the PHMS backbone. This reactive group then serves as an initiator for the growth of zwitterionic methacrylate polymer side chains via atom transfer radical polymerization (ATRP). The length of these ionic polymer grafts as well as the types of ionic groups in them are controlled by changing the types and molar ratios of the monomers used in the reaction. Additionally, nonionic but hydrophilic monomers such as hydroxyethylmethacrylate (HEMA) or dimethylaminoethyl methacrylate (DMAEMA) can be incorporated during polymerization.

Further, charged polymers were added to the immobilization material reaction mixture during crosslinking. The charged polymers can be a polymer containing a group that is positively or negatively charged or both. For example, a positively charged polymer can be poly(diallyldimethylammonium chloride)[polyquaternary ammonium] (PQA) and a negatively charged polymer can be a sodium salt of poly(acrylic acid) (PAA-Na). A person of skill in the art would have known that other polymers containing amine groups or acid groups would be suitable for this purpose. For example, polymers containing a primary amine, a secondary amine, a tertiary amine, a quaternary amine, a carboxylic acid, a phosphonic acid, a sulfonic acid, a sulfuric acid, a phosphoric acid, a sulfamic acid, or a combination thereof could be used. These polymers become entangled in the immobilization matrix during cross-linking. These charged polymers can be added to the crosslinking mixture at a concentration of from 5 wt. % to 30 wt. %, 10 wt. % to 30 wt. %, 15 wt. % to 25 wt. % or 20 wt. % to 25. wt. % based on the total weight of components of the immobilization matrix.

### Encapsulated Enzyme Packing Materials

The immobilized enzyme can be incorporated into various packing materials including pellets, coatings, or films. In some cases, when the immobilized enzyme is coated on an inert support, the inert support may be modified to facilitate adhesion of the immobilized enzyme.

After adding a crosslinking catalyst to the enzyme/micellar polymer mixture, this liquid can be transferred into a mold for the allotted time to allow the enzyme-containing micellar polymer to crosslink into a rubbery material. These molds can contain any number of differently sized and/or shaped voids to produce a variety of geometries for the pellets.

Alternately, after the crosslinking catalyst is added, the enzyme/micellar polymer mixture can be sprayed to form small beads of enzyme-containing polymer packing. The size of these beads is affected by solution viscosity and sprayer velocity.

Before adding the crosslinking catalyst, the enzyme/micellar polymer mixture is a liquid and can be applied to a variety of supports as a film or coating. For example, three-dimensional supports such as random or dumped packing can be tumble coated, spray coated, or dip coated with the enzyme/micellar polymer liquid mixture followed by addition of the crosslinking catalyst to form the film or coating on the packing material. Structured packing can also be coated using the same techniques to create a higher void volume in the absorber column. The film thickness is affected by the solution viscosity in the dip coating or spray coating or the amount of enzyme/polymer mixture used per given volume of packing in tumble coating. The film thickness and specific surface area of the packing are two important variables in determining the maximum enzyme loading in the absorber unit.

Initial experiments in coating various supports showed that the immobilized enzyme did not adhere well to the substrate and could be easily removed by agitation. To improve the adhesion, surface functionalization of packing materials (e.g., ceramics, graphite, stainless steel, plastics) with groups capable of coupling to the micellar polymer coating during crosslinking can be performed.

Scheme 15 shows functionalization of ceramic or graphite-based support materials. The support materials are etched to introduce surface hydroxide groups, then isocyanate groups are produced and coupled to disilanol-terminated PDMS. The surface silanol groups react with the micellar polymer layer during crosslinking to covalently attach the immobilized enzyme to the support. These surfaces can also be functionalized with alkoxy-silane groups or allyl groups to react with the micellar polymer by the reactions in Scheme 35. The R groups can be alkyl.

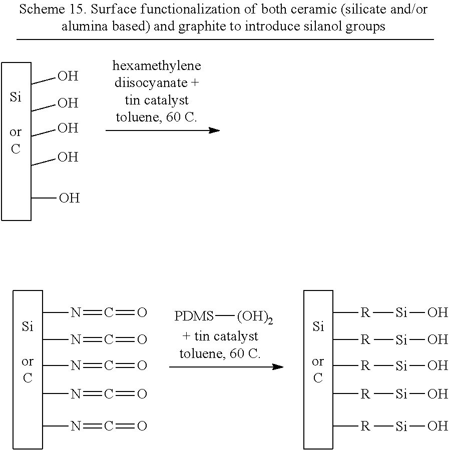

Surface functionalization of the support materials was confirmed using attenuated total reflectance Fourier transform infrared spectroscopy (ATR FTIR). Since the reaction between isocyanates and silanols is catalyzed by the same tin catalysts used during the crosslinking reaction, it may be possible to functionalize the surface with isocyanate groups only. The isocyanate groups could then react with the silanol groups in the enzyme micellar polymer mixture during crosslinking to covalently attach the enzyme and micellar polymer to the support.

Commercially available plastics such as poly(methyl methacrylate), cellulose, or polyurethanes (Banuls, M. J.; Gonzalez-Pedro, V.; Puchades, R.; Maquieira, A. Bioconjugate Chemistry 2007, 18, 1408-1414; Ly, E. B.; Bras, J.; Sadocco, P.; Belgacem, M. N.; Dufresne, A.; Thielemans, W. Materials Chemistry and Physics 2010, 120, 438-445; Tan, K.; Obendorf, S. K. Journal of Membrane Science 2006, 274, 150-158) can be similarly functionalized with isocyanate groups and then disilanol-terminated PDMS according to published procedures. Several commercially available plastic materials can be etched and/or oxidized, including polystyrene, polypropylene/polyethylene (PP/PE), or fluoropolymers (e.g., poly(tetrafluoroethylene) (PTFE), and poly(ethylene terephthalate)). For instance, structured packing is commercially available made from a variety of plastics, including PP, poly(vinyl chloride (PVC), poly(vinylidene fluoride) (PVDF), and poly(ether ether ketone) (PEEK). Due to the presence of functional moieties in the backbone capable of being chemically etched and modified, PVC and PEEK can be desirable plastic substrates for enzymes immobilized in micellar polymer films.

After acid pickling or electrochemical reduction to remove the surface oxide layer, stainless steel can be functionalized with a variety of amine or sulfur-containing compounds that coordinate with the exposed metal atoms of the freshly etched steel surface. (Ruan, C. M.; Bayer, T.; Meth, S.; Sukenik, C. M. Thin Solid Films 2002, 419, 95-104) Aminoethylaminopropyltrimethoxysilane (AEAPTMS) functionalizes steel with methoxysilane groups that can couple to the silanol groups of the micellar polymer mixture during crosslinking Aminoethylaminopropylsilsesquioxane-methylsilsesquioxane (AEAPSSQ-MSSQ) copolymer functionalizes steel with silanol groups that can react with silicon hydride groups during crosslinking. The IR peaks for Si—O—Si bonds show surface functionalization by the amine-siloxane. Since these IR peaks were still present after extensive sonication in solvents in which they were readily soluble, both AEAPTMS and AEAPSSQ-MSSQ are covalently attached to the steel surface.

Ceramic and graphite packings can be functionalized with —Si—OH groups that further require with allyl trichlorosilane to produce allyl groups on the surface of the packing material that can react with the micellar polymer during crosslinking.

Unless otherwise indicated, the alkyl groups described herein are preferably lower alkyl containing from one to eight carbon atoms in the principal chain and up to 20 carbon atoms. Alkyls may be substituted or unsubstituted and straight or branched chain. Examples of unsubstituted alkyls include methyl, ethyl, n-propyl, i-propyl, n-butyl, i-butyl, s-butyl, t-butyl, n-pentyl, i-pentyl, s-pentyl, t-pentyl, and the like. The term “substituted,” as in “substituted alkyl,” means that various heteroatoms such as oxygen, nitrogen, sulfur, phosphorus, and the like can be attached to the carbon atoms of the alkyl group either in the main chain or as pendant groups. For example, the substituted alkyl groups can have —C—X—C— fragments in the main chain wherein the X is a heteroatom. Further, the substituted alkyl groups can have at least one hydrogen atom bound to a carbon atom replaced with one or more substituent groups such as hydroxy, alkoxy, alkylthio, phosphino, amino, halo, silyl, nitro, esters, ketones, heterocyclics, aryl, and the like.

The term “aryl” as used herein alone or as part of another group denotes an optionally substituted monovalent aromatic hydrocarbon radical, preferably a monovalent monocyclic or bicyclic group containing from 6 to 12 carbons in the ring portion, such as phenyl, biphenyl, naphthyl, substituted phenyl, substituted biphenyl or substituted naphthyl. Phenyl and substituted phenyl are the more preferred aryl groups. The term “aryl” also includes heteroaryl.

The term “-ene” as used as a suffix as part of another group denotes a bivalent radical in which a hydrogen atom is removed from each of two terminal carbons of the group, or if the group is cyclic, from each of two different carbon atoms in the ring. For example, alkylene denotes a bivalent alkyl group such as methylene (—CH₂—) or ethylene (—CH₂CH₂—), and arylene denotes a bivalent aryl group such as o-phenylene, m-phenylene, or p-phenylene. For clarity, addition of the -ene suffix is not intended to alter the definition of the principal word other than denoting a bivalent radical. Thus, continuing the example above, alkylene denotes an optionally substituted linear saturated bivalent hydrocarbon radical.

The term “hydrocarbon” as used herein describes a compound or radical consisting exclusively of the elements carbon and hydrogen.

The term “substituted” as in “substituted aryl,” “substituted alkyl,” and the like, means that in the group in question (i.e., the alkyl, aryl or other group that follows the term), at least one hydrogen atom bound to a carbon atom is replaced with one or more substituent groups such as hydroxy (—OH), alkylthio, phosphino, amido (—CON(R_{A})(R_{B}), wherein R_{A} and R_{B} are independently hydrogen, alkyl, or aryl), amino(—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like. When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

Having described the invention in detail, it will be apparent that modifications and variations are possible without departing from the scope of the invention defined in the appended claims.

### EXAMPLES

The following non-limiting examples are provided to further illustrate the present invention.

### Example 1

### Phase Behavior of PHMS-g-PEG Micellar Polymers

Synthesis of Sample 1 (29 Wt. % PEG) Sample for SEM.

A micellar polymer mixture was made with the following components: 3 mL of PHMS-g-PEG (62 wt. % PEG), 3 mL of PHMS-g-PEG (71 wt. % PEG), 3 mL of PHMS-g-PEG (82 wt. % PEG), 9 mL of disilanol-terminated PDMS 4200 g/mol, 2 mL of PHMS 2250 g/mol, and 2 mL of a 50 wt. % solution of silanol trimethylsilyl-modified Q resin in decamethylcyclopentasiloxane. This micellar polymer mixture has 29 wt. % PEG overall.

The above micellar polymer mixture (3 mL) was vortexed with 0.1 mL of dibutyldilauryltin crosslinking catalyst in a glass vial until thoroughly mixed. This solution was then transferred to an acrylic mold (1 inch×1 inch×¼ inches deep) and annealed in an oven at 50° C. for 30 minutes.

Synthesis of Sample 2 (32 wt. % PEG) Sample for SEM.

A micellar polymer mixture was made with the following components: 3 mL of PHMS-g-PEG (62 wt. % PEG), 3 mL of PHMS-g-PEG (71 wt. % PEG), 3 mL of PHMS-g-PEG (82 wt. % PEG), 9 mL of disilanol-terminated PDMS 4200 g/mol, 0.6 mL of PHMS 2250 g/mol, and 1.4 mL of a 50 wt. % solution of silanol trimethylsilyl-modified Q resin in decamethylcyclopentasiloxane. This micellar polymer mixture has 32 wt. % PEG overall.

To directly image its hydrophilic regions and determine their size, distribution, and polydispersity; PHMS-g-PEG micellar polymers were stained with a phosphotungstic acid (PTA) solution according to published procedures and imaged via scanning electron microscopy (SEM). (Ferrer, G. G.; Sanchez, M. S.; Gomez Ribelles, J. L.; Romero Colomer, F. J.; Monleon Pradas, M. European Polymer Journal 2007, 43, 3136-3145; Du, W.; Li, Y.; Nystrom, A. M.; Cheng, C.; Wooley, K. L. Journal of Polymer Science Part A: Polymer Chemistry 2010, 48, 3487-3496) As PTA is a polar, water soluble salt, it preferentially partitions into the hydrophilic PEG domains of the PHMS-g-PEG micellar polymer network, where the tungsten (W) creates contrast for electron micrograph collection. To perform the analysis, bulk pieces of polymer were first cut with a razor blade so that all of the edges were removed and then sectioned into thirds. This was done to open up the bulk material to staining and subsequent imaging. After exposure to a 1% (w/v) aqueous PTA solution for 12 hours, a polymer section was sliced with a scalpel, creating a thin cross-section, which was mounted onto an SEM substrate using two-sided carbon tape.

### Example 2

### SEM of PHMS/PDMS Polymer

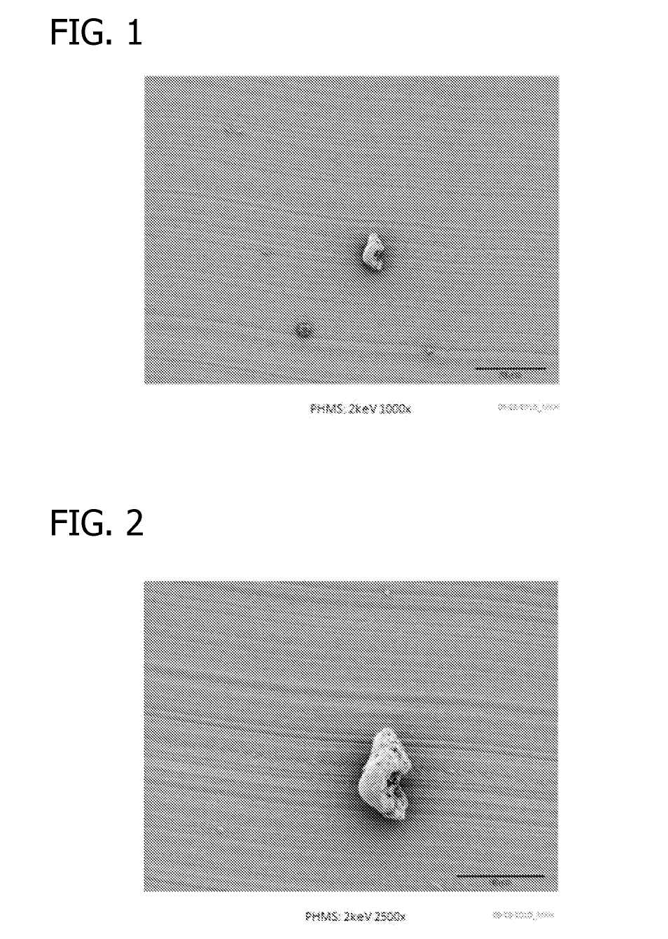

A polymer sample made exclusively of PHMS crosslinked with disilanol-terminated polydimethylsiloxane (PDMS) was created as a control against the PHMS-g-PEG micellar polymer samples. FIG. 1 shows a 1000× magnification electron micrograph of a cross-section of the polysiloxane-only polymer. As can be seen in the figure, the polymer appears to have a smooth, striated texture, with no evidence of any phase-separated, micellar features.

FIG. 2 shows a 2500× magnification electron micrograph of the polysiloxane-only polymer sample, again depicting the smooth, striated surface of the polymer. A piece of debris was used as a focal point for image focusing.

### Example 3

### SEM of PHMS-g-PEG Micellar Polymer

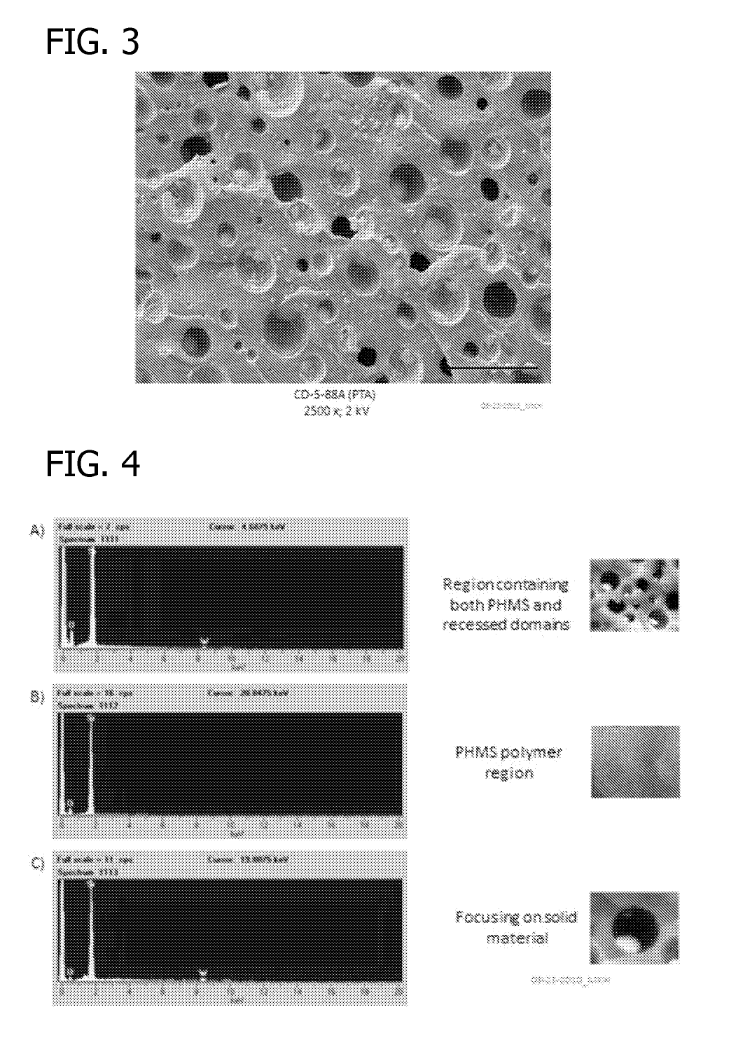

A representative sample (Sample 1) of the crosslinked PHMS-g-PEG micellar polymer mixture (29 wt. % PEG overall) is shown in FIG. 3 at 2500× magnification. This electron micrograph clearly shows the formation of a several hemispherical regions within the polymer matrix with a brightly contrasting material (tungsten) collecting within them.

The bright contrasting material appearing in the recessed areas was proven by x-ray analysis to be tungsten. First a region containing both the polysiloxane matrix and the recessed regions was analyzed indicating the presence of silicon, oxygen, and tungsten (FIG. 4A). Next, the microscope was focused on a region between the recessed domains where only the polysiloxane polymer resides. In this electron micrograph analysis (FIG. 4B), the peaks for oxygen and silicon were present, but the peak for tungsten was not. Finally, the microscope was focused directly on a recessed region containing a large piece of the solid material (FIG. 4C). The x-ray analysis once again showed the presence of oxygen, silicon, and tungsten. The reappearance of the tungsten peak in this spectrum confirmed that the high contrast material in the recessed regions is in fact tungsten.

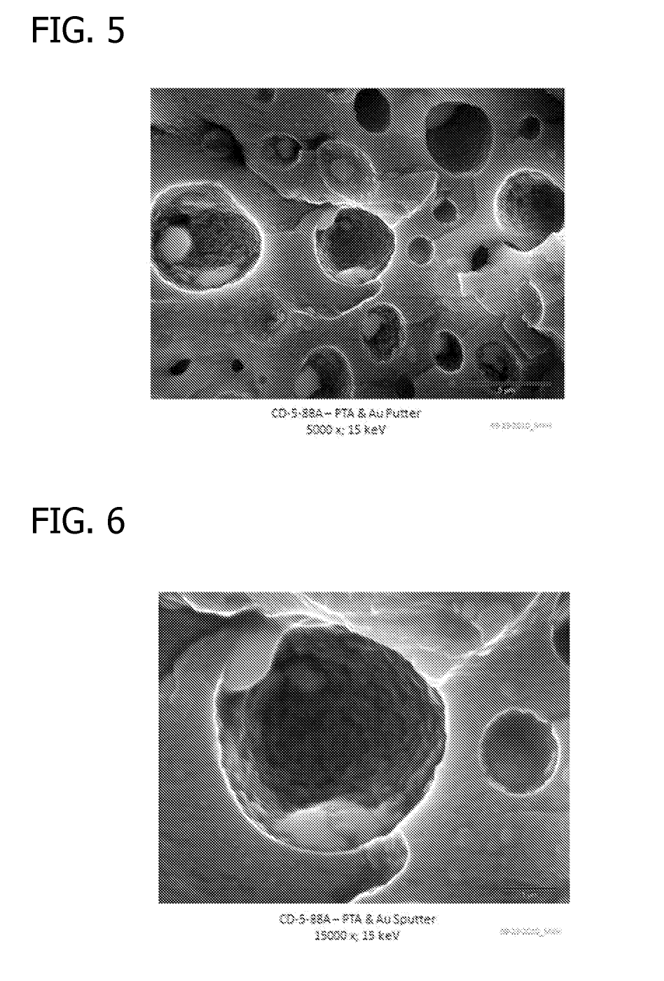

In an attempt to image with higher resolution, the sample was sputtered with a thin layer of Au, which made the material surface much more conductive, reduced sample charging, and allowed electron micrographs to be taken at higher magnifications with higher accelerated potentials. FIG. 5 shows a 5000× magnification electron micrograph of the Au coated sample using a 15 keV acceleration voltage. In these electron micrographs, the difference in surface texture between the interior of the recessed domains and the surface of the PHMS can be seen more clearly. The large solid pieces inside the micelles were tungsten. The varying depth of the recessed domains can also be seen more clearly. FIG. 6 shows a 15,000× magnification electron micrograph of the recessed domain sitting in the middle of FIG. 5.

Based on the above electron micrographs, it can be seen that the inclusion of PEG into the polymer matrix resulted in the formation of phase-separated domains not found in the PHMS/PDMS control sample. The association of tungsten with the recessed domains of the polymer further supported that the region is hydrophilic (i.e., PEG-rich) and phase separated.

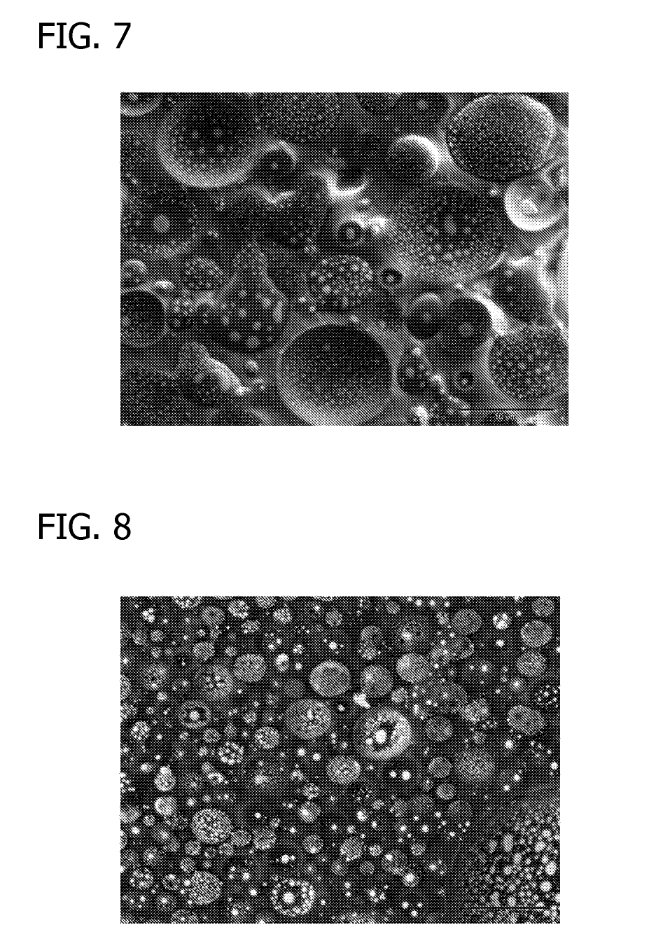

A series of different polymer samples were prepared and imaged using the same techniques described above. Variables such as crosslinking temperature, crosslinking density, and water content were varied to determine their effect on the polymer morphology. One particular crosslinked PHMS-g-PEG micellar polymer mixture (32 wt. % PEG overall) was prepared using less of the silanol Q crosslinking resin and less PHMS, resulting in a lower crosslinking density. FIG. 7 shows a 2,500× magnification of the polymer sample after staining with 1% (w/v) PTA. While the large, hemispherical domains seen in previous samples were still present, small discrete regions were apparent within them, as indicated by the tungsten staining.

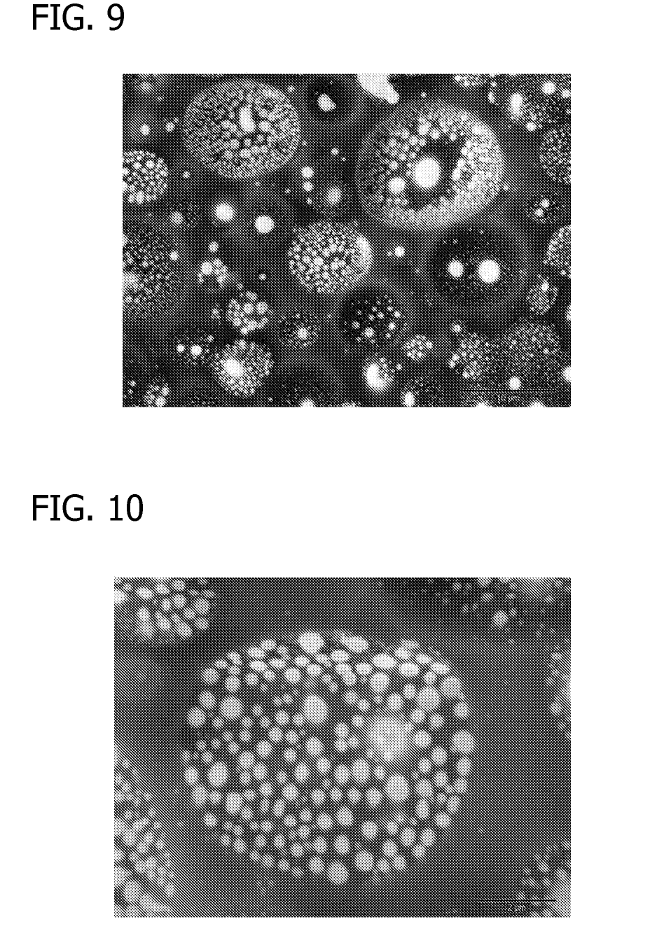

To create a greater contrast between the tungsten and the polymer, a backscattering electron (BSE) detector was used. The phenomenon of electron backscattering is significantly increased in heavy atoms (i.e. tungsten). As a result, areas containing heavy atoms appear significantly brighter than areas without. FIG. 8 and FIG. 9 show a 1,000× and 2,500× magnification (respectively) electron micrograph of the same crosslinked PHMS-g-PEG micellar polymer mixture (32 wt. % PEG overall; lower crosslink density) using a BSE detector. The electron micrographs clearly revealed the presence of tungsten in small, discrete domains within the larger hemispherical recessions.

FIG. 10 shows a 10,000× magnification electron micrograph focusing on one of the larger domains (˜5 μm). In this electron micrograph it is clear that the discrete tungsten-stained regions were sub-micron in diameter, appearing to range in size from tens to hundreds of nm. It is worth noting that the range of size distribution could be an artifact of the tungsten staining technique; some of the larger areas could be caused by the accumulation of excess tungsten.

SEM with PTA staining showed that the crosslinked PHMS-g-PEG micellar polymer mixtures contained a phase-separated, micellar architecture. The PHMS/PDMS (polysiloxane only) control sample consisted of a smooth textured surface with no evidence of any phase-separated, or micellar, features. However, all of the PHMS-g-PEG micellar polymer mixtures imaged via SEM contained visible, recessed features in the bulk material. The association of tungsten in these recessed regions further support that these polymers are phase-separated by indicating that these regions are made up of a hydrophilic, PEG-rich domains. The fact that the tungsten stain did not affect any regions in the PHMS/PDMS sample or any of the regions surrounding the recessed features in the PHMS-g-PEG samples indicated that the staining technique is selective for hydrophilic domains. It was also determined that lowering the crosslinking density has an effect on the morphology of the PHMS-g-PEG polymer that created a distribution of tens to hundreds of nanometer-sized PEG-rich domains within the larger, micron-sized features of the polymer.

The observed sizes of these hydrophilic regions within the PHMS-g-PEG micellar polymers analyzed via SEM were larger than a typical monomeric carbonic anhydrase enzyme, however. For example, recently published data for bovine carbonic anhydrase II (bCAII) determined a hydrated enzyme size of approximately 6.7 nm×6.7 nm×12 nm. (Saito, R.; Sato, T.; Ikai, A.; Tanaka, N. Acta Crystallographica 2004, D60, 792-795) As such, enzyme leaching from these PHMS-g-PEG micellar polymers, particularly from the larger hydrophilic pockets, could have reduced the effective lifetime of the immobilized enzyme packing material in a carbon dioxide absorber unit.

### Example 4

### PEG Derivatives that React with the Amino Group of CA

### Example 4(a)

### PEG Epoxides

PEG epoxides were purchased from Creative PEG Works or synthesized using a modified literature procedure (Kaul, G.; Amiji, M., Pharmaceutical Research 2002, 19, 1061-1067).

The bioconjugation reaction of PEG-550 Da epoxide with bovine carbonic anhydrase II (bCAII) occurred at a very slow rate. After performing the reaction at different pHs and temperatures, it was concluded that the best conditions for the bioconjugation reaction between PEG epoxides and bCAII was high pH, such as 11, and high temperature, such as 35-40° C.

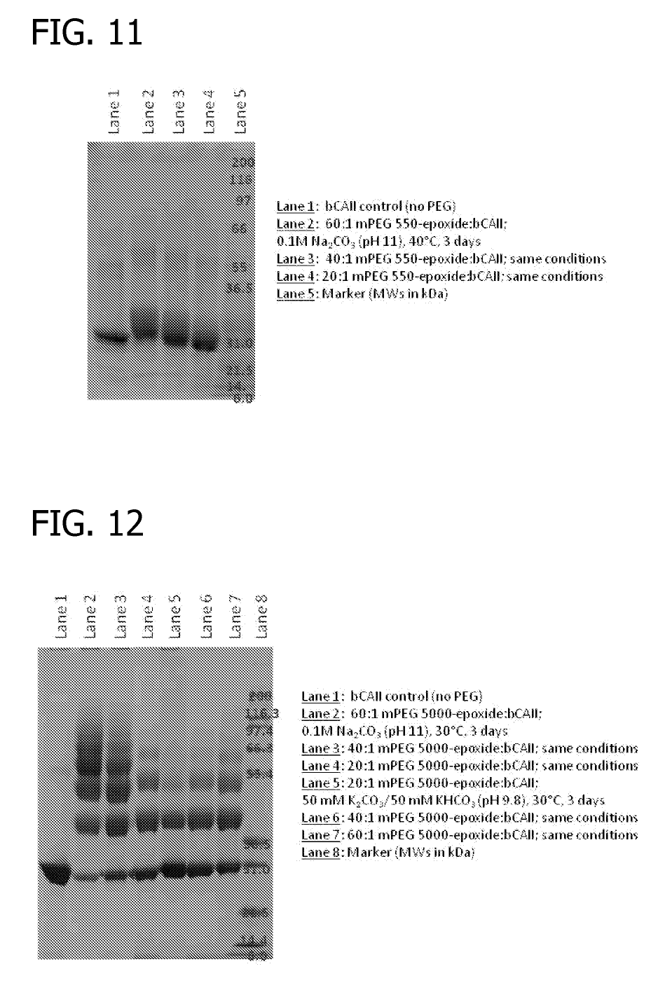

FIG. 11 shows the sodium dodecyl sulfate polyacrylamide gel electrophoresis (SDS PAGE) results for the bioconjugation reaction between PEG-550 Da epoxide and bCAII. There was movement on the gel where there was broadening of the band. Also, the bCAII enzyme was not completely functionalized. Because the SDS PAGE provided a very quick way to determine if the bioconjugation reaction occurred, higher molecular weight PEGs were studied, as their movement on the gel was very clear. Thus, PEG-5000 Da epoxide was purchased from Creative PEG Works and used in the bioconjugation reactions. The SDS PAGE results for the bioconjugation reaction of PEG-5000 Da epoxide and bCAII at 30° C. at pH 9.8 and pH 11 are compared in FIG. 12. With the larger molecular weight PEGs, the bands were clearly more defined and one could tell unambiguously if the PEGylation reaction occurred.

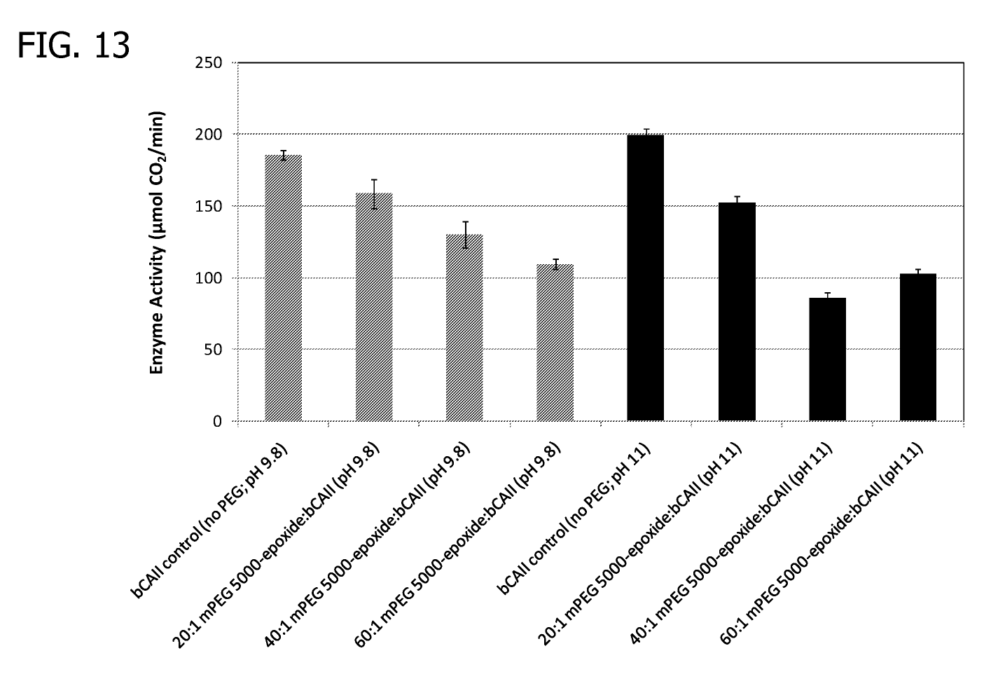

Overall, it was determined that conditions of higher pH and temperature favored the bioconjugation reaction between bCAII and PEG epoxides. However, since the reaction was very slow and there was considerable loss of enzyme activity, the epoxide moiety was not the most desirable chemistry for tethering CA into the micellar polymer (FIG. 13).

### Example 4(b)

### PEG Sulfonate Esters

The nosylate (4-nitrophenylsulfonyl) and tosylate (4-toluenesulfonyl) functional groups were identified as suitable groups for attachment at the end of polyethylene glycol (PEG) chains and subsequent grafting to polysiloxane-based micellar polymers. This rationale was based on the fact that these groups are excellent leaving groups in solution-based organic chemistry. Also, the chemistry for grafting these groups onto the polysiloxane backbone was published in the literature.

A 500 ml 3-necked round bottomed flask was charged with poly(ethylene glycol monomethyl ether) methoxyPEG-550 Da (50 g, 0.09 mol) and 200 ml of chloroform. The resulting solution was cooled to 0° C. To an addition funnel was charged para nitro sulfonyl chloride (nosyl chloride), (30.2 g, 0.14 mol) in 75 ml of chloroform. Powdered sodium hydroxide (14.5 g, 0.36 mol) was added to the PEG solution. After 10 minutes, the nosyl chloride was added dropwise over 30 minutes. After the addition was complete, the reaction mixture was allowed to stir at 0° C. for 2 hours. The bath was removed and the reaction mixture was stirred at room temperature for an additional 16 hours.

The reaction mixture was filtered and the filtrate was treated with pyridine (50 ml) and the solution was stirred at room temperature for 2 hours. The mixture was transferred to a reparatory funnel and washed with 5% HCl (3×200 ml), followed by washing with water (1×200 ml), and finally with brine (1×200 ml). The organic layer containing the product was then dried over anhydrous magnesium sulfate, filtered, and evaporated under reduced pressure to give 48 g (84% yield) of the product as an amber oil. ¹H NMR (CDCl₃) δ 8.38 (d, J=10.00 Hz, 2H) 8.13 (dd, J=10.00 Hz, 2H), 4.28 (m, 2H), 3.78 (m, 2H), 3.63 (m, 44H), 3.36 (s, 3H).

The procedure was straightforward for lower molecular weight PEGs, but had to be modified by changing the solvent and base used due to solubility issues for larger molecular weight PEGs. Changing the solvent was also important for isolation of the higher molecular weight PEGs. The derivatives synthesized were the mPEG-550 Da nosylate as well as the mPEG-550, 1900, and 5000 Da tosylates.

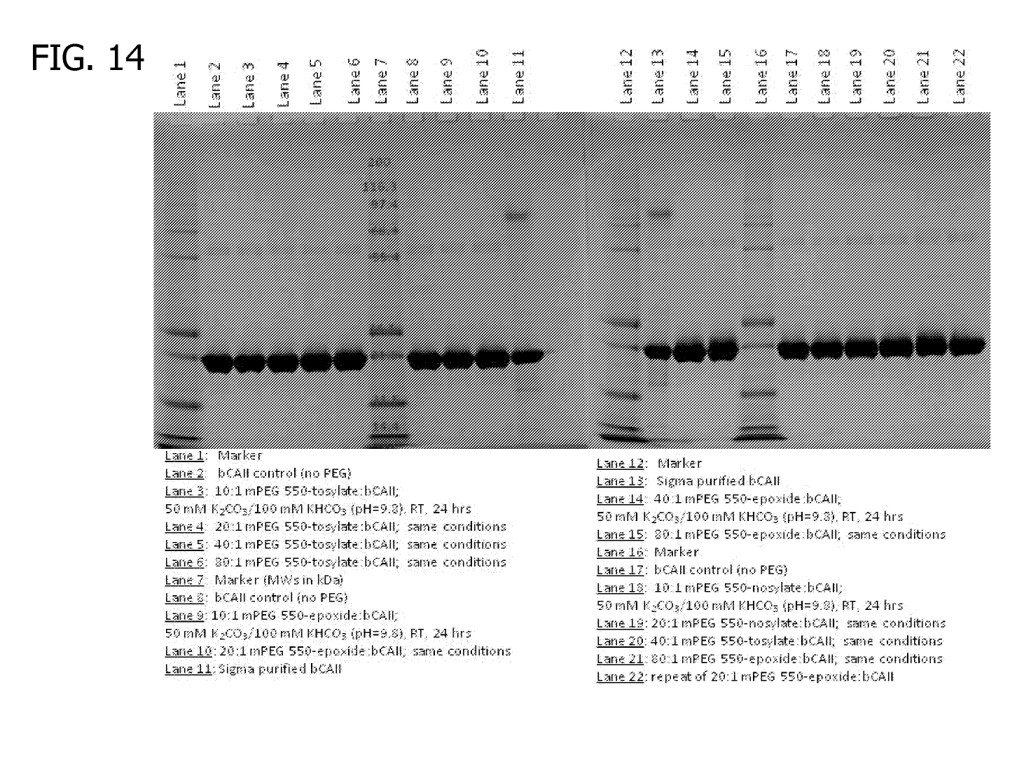

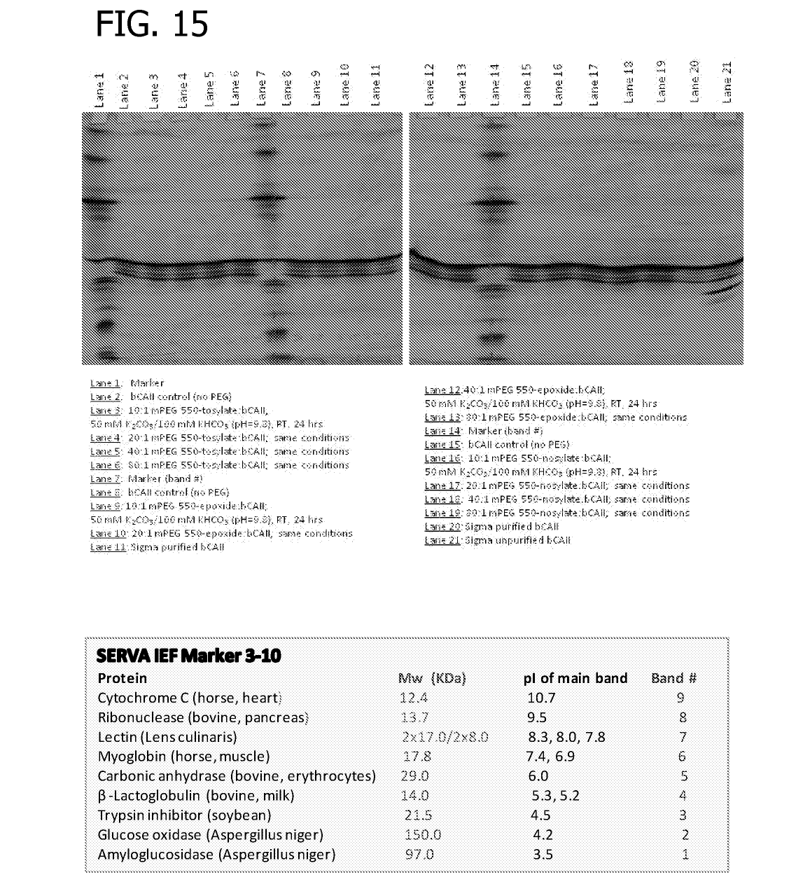

FIG. 14 shows the SDS PAGE results for the reactions between PEG-550 Da nosylate, tosylate, and epoxide with bCAII at various ratios for 24 hours at pH 9. Visually nothing seemed to have happened under these reaction conditions. If a reaction had occurred, it would have been difficult to determine because addition of PEG 550 Da to the enzyme of molecular weight 30 KDa did not provide a large enough molecular weight increase to significantly move the protein on the gel. Thus, larger molecular weight PEGs were either synthesized or purchased.

Since not much information could be obtained from the SDS PAGE results using mPEG 550 Da, isoelectric focusing (IEF) gels were also performed using these samples and the reactivity of functionalized PEGs with enzymes under these reaction conditions was investigated. Those results are shown in FIG. 15. As with the SDS PAGE results, these reactions showed no differences between native bCAII and bCAII exposed to these functionalized PEGs at any molar ratios, and indicated that the nosyl and tosyl functionalized PEGs did not couple to the enzymes under these reaction conditions.

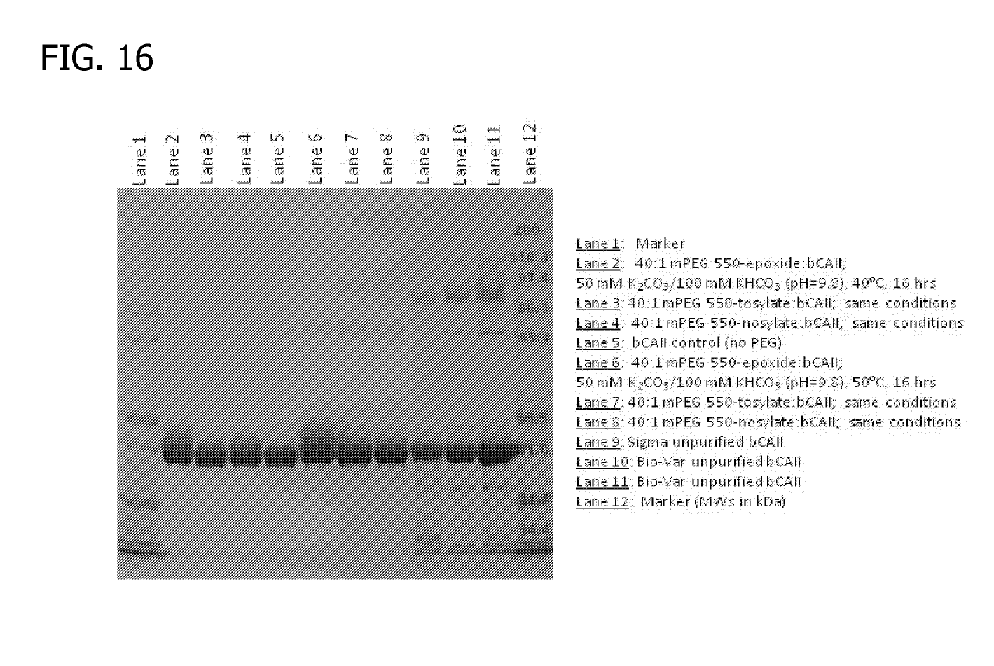

Literature reports recommended elevated temperatures for these reactions. Two different reaction series were performed, one at 40° C. and the other at 50° C., at the same molar ratios of functionalized PEG to CA. The SDS PAGE results of those two series are highlighted in FIG. 16. FIG. 16 showed a broadening of the protein band towards higher molecular weights with the bCAII enzymes exposed to functionalized PEGs, particularly the epoxide, both at 40° C. (Lanes 2-4) and 50° C. (Lanes 6-8) as compared to the control (Lane 5). This result suggested that the functionalized PEGs could have coupled to bCAII at these reaction conditions and caused an increase in the overall molecular weight of the protein band.

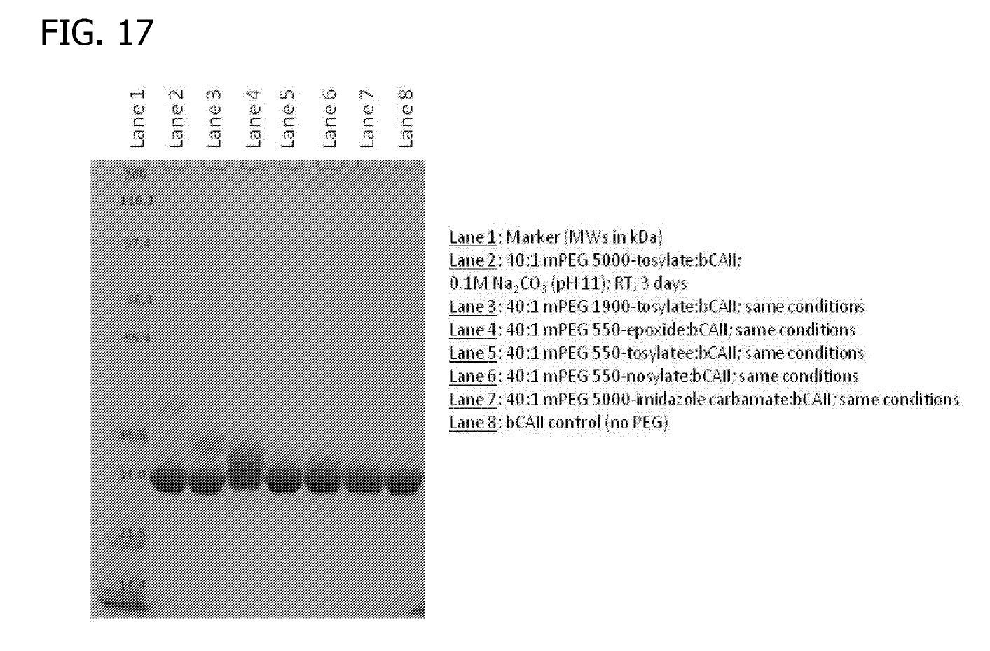

A new series of reactions with these functional moieties were set up at a higher pH (11) for a longer time (3 days) to determine if bioconjugation occurred in higher yield under these conditions. The SDS PAGE results are shown in FIG. 17. The bioconjugation reaction provided a very low yield, even after 3 days at room temperature at pH 11. Even under high pH and elevated temperatures, the coupling reaction between bCAII and PEG nosylates and tosylates was extremely sluggish. Several different reaction conditions were tried and no reaction occurred. Additionally, other CA isoforms like human carbonic anhydrase IV (hCAIV) were also tried, and there no significant bioconjugation occurred. Based on these results, the tosylate and nosylate PEGS were found less than optimal for tethering CA into the micellar polymer.

As discussed earlier, the nosylate and tosylate functionalized PEGs did not readily undergo bioconjugation with bCAII, as the reactions were very slow. Next, mPEG mesylates and tresylates were studied. The mPEG-5000 Da mesylate was purchased from Creative PEG Works in South Carolina, while mPEG-5000 Da tresylate was synthesized according to a literature procedure where temperature, solvent, and product isolation where modified to improve yield. The reaction conditions for the bioconjugation of mPEG-5000 Da mesylate with bCAII that were performed are shown in Table 4.1.

TABLE 4.1
Reaction Conditions for Bioconjugation between
mPEG 5000 Mesylate and bCAII

| | mPEG- | | | | Enzyme |
|---|---|---|---|---|---|
| Sample # | 5000 Da | Ratio | Buffer | Temperature, Time | (4 mg) |
| TA57-65-1 | Mesylate | 20:1 | 50 mM K₂CO₃/50 mM | 30° C., 3 days | bCAII |
| | | | KHCO₃, pH | | |
| | | | 9.8 | | |
| TA57-65-2 | Mesylate | 40:1 | 50 mM K₂CO₃/50 mM | 30° C., 3 days | bCAII |
| | | | KHCO₃, pH | | |
| | | | 9.8 | | |
| TA57-65-3 | Mesylate | 60:1 | 50 mM K₂CO₃/50 mM | 30° C., 3 days | bCAII |
| | | | KHCO₃, pH | | |
| | | | 9.8 | | |
| TA57-65-4 | Mesylate | 20:1 | 0.1M Na₂CO₃, pH | 30° C., 3 days | bCAII |
| | | | 11 | | |
| TA57-65-5 | Mesylate | 40:1 | 0.1M Na₂CO₃, pH | 30° C., 3 days | bCAII |
| | | | 11 | | |
| TA57-65-6 | Mesylate | 60:1 | 0.1M Na₂CO₃, pH | 30° C., 3 days | bCAII |
| | | | 11 | | |

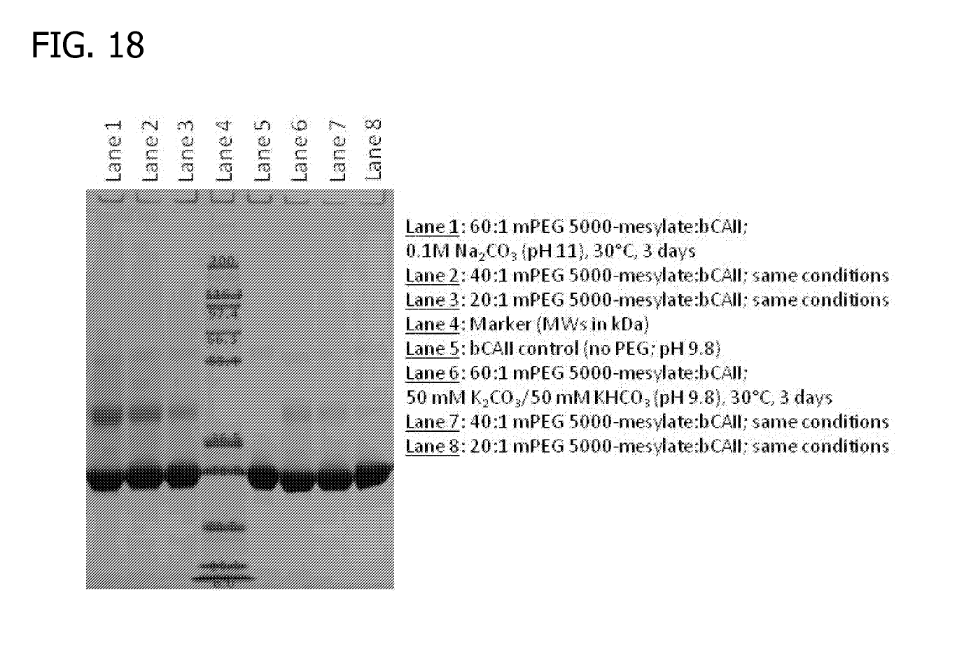

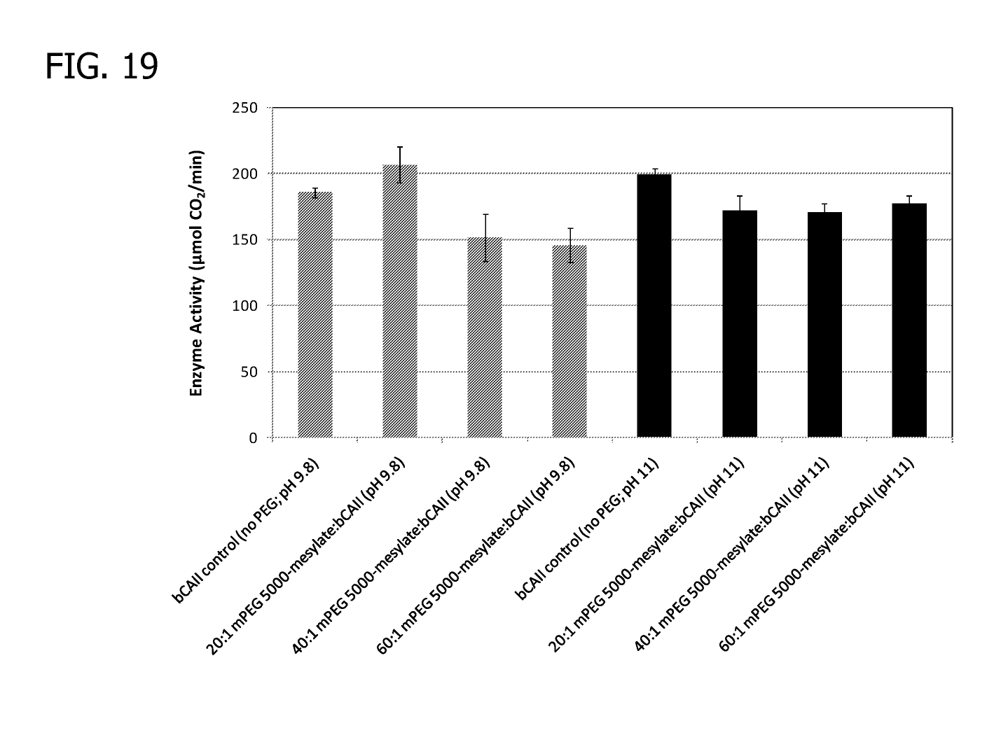

After 3 days at 30° C., SDS PAGE results showed that the reactions were incomplete (FIG. 18). These results showed the bioconjugation reaction was too slow. Enzyme activity was tested using the pH stat, shown in FIG. 19; the observed activity was attributed both to the native enzyme (as the reactions were incomplete) and to the PEGylated enzyme.

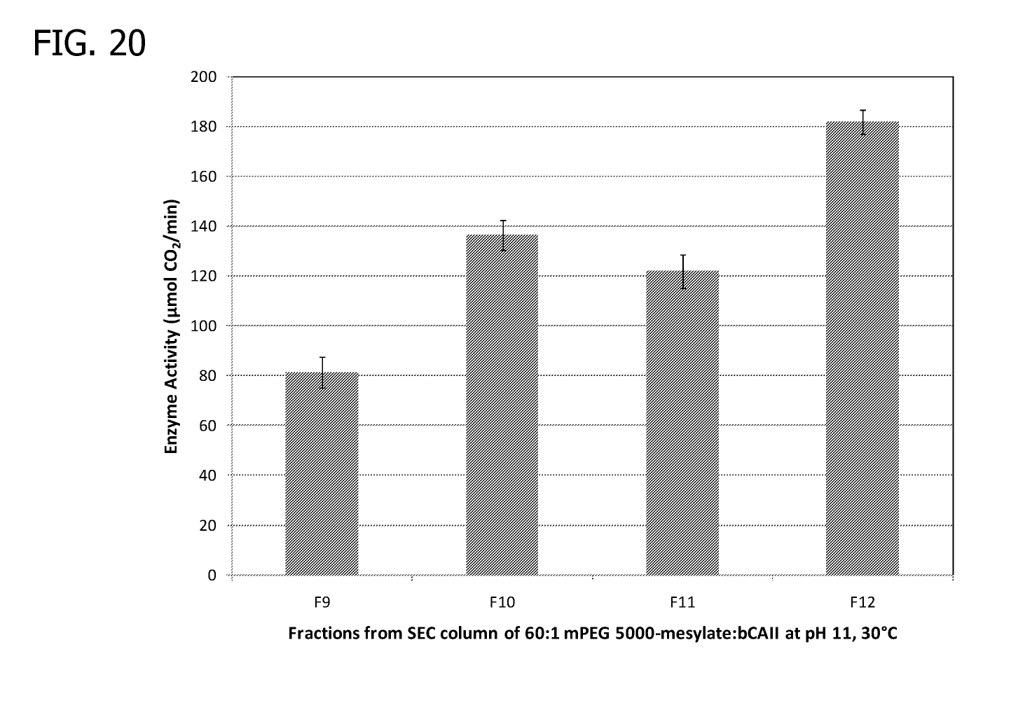

Using a preparatory size exclusion (SEC) column, fractions from the reaction of 60:1 mPEG 5000 Da-mesylate:bCAII at pH 11 were isolated, and enzymatic activity of these fractions measured. The activity of the collected fractions is shown in FIG. 20. Fraction F10, which according to SDS PAGE had predominately monoPEGylated enzyme (Lane 4, FIG. 21), showed ˜75% activity retention upon bioconjugation.

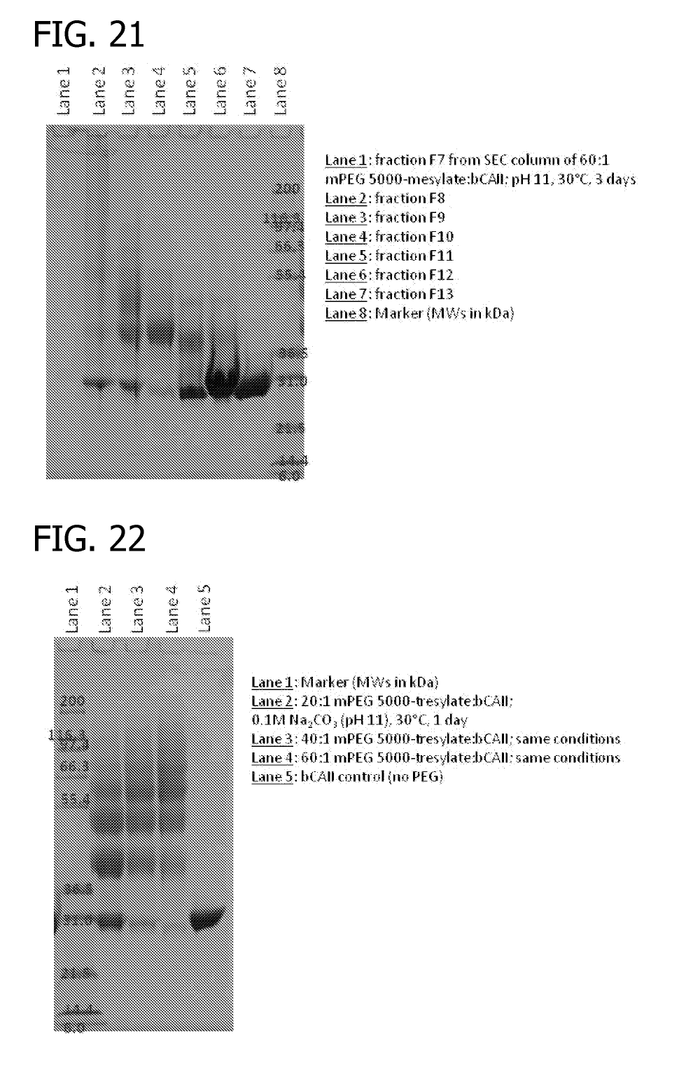

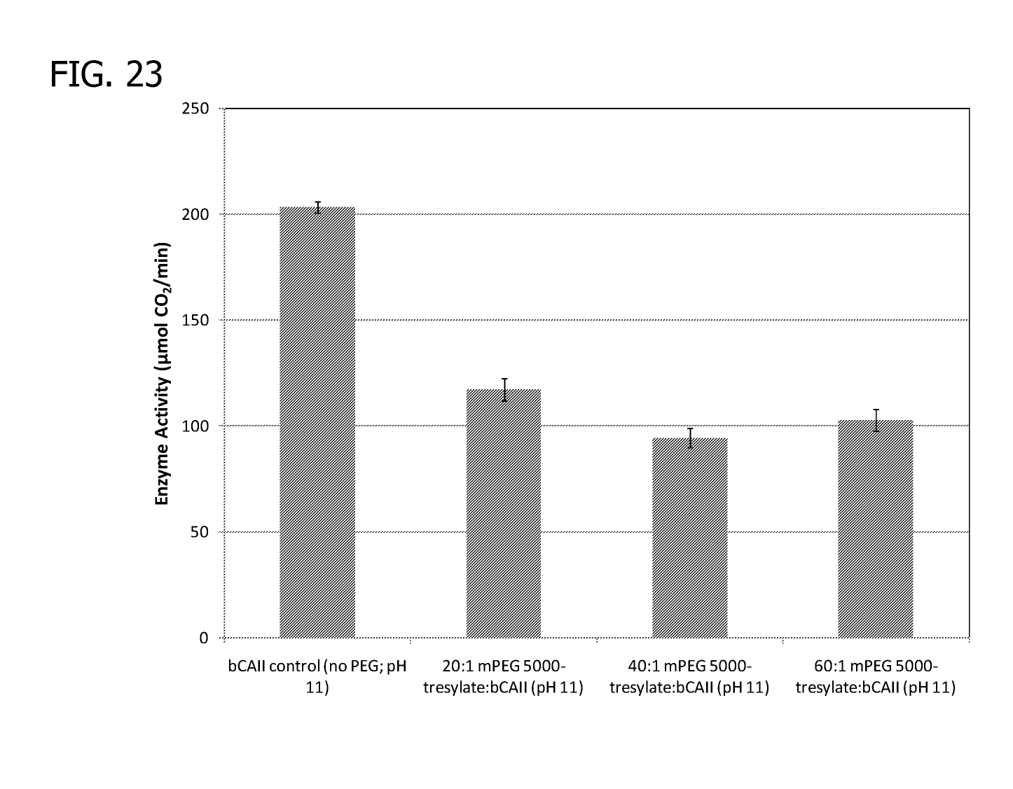

Next, the bioconjugation reaction between bCAII and mPEG-5000 Da tresylate was evaluated. The reaction conditions are shown in Table 4.2. The initial results on the SDS PAGE gel were very encouraging. The enzyme was almost completely PEGylated at a PEG tresylate:bCAII ratio of 40:1 or 60:1 (FIG. 22, lane 3 and 4). According to the pH stat activity measurement, there was about 50% retention of enzymatic activity after bioconjugation (FIG. 23). These results demonstrated a successful bioconjugation reaction between the CA amino group and PEG derivative with retention of enzymatic activity.

TABLE 4.2
Reaction Conditions for Bioconjugation between
mPEG 5000 Tresylate and bCAII

| | mPEG- | | | Temperature, | Enzyme |
|---|---|---|---|---|---|
| Sample # | 5000 Da | Ratio | Buffer | Time | (4 mg) |
| CD-5-164A | Tresylate | 20:1 | 0.1M Na₂CO₃, | 30° C., 24 h | bCA II |
| | | | pH 11 | | |
| CD-5-164B | Tresylate | 40:1 | 0.1M Na₂CO₃, | 30° C., 24 h | bCA II |
| | | | pH 11 | | |
| CD-5-164C | Tresylate | 60:1 | 0.1M Na₂CO₃, | 30° C., 24 h | bCA II |
| | | | pH 11 | | |

Ionic strength of buffer, pH, temperature, reaction times, and ratio of PEG to enzyme were factors for optimization of enzymatic activity with the PEG tresylate.

### Example 4(c)

### PEG Succinimide Esters

Another functional group considered for tethering the bCAII enzyme onto the polyethylene glycol (PEG) chain of the polymer was a succinimide ester. PEG-NHS valerate (SVA) and PEG-NHS propionates (SPA) were investigated as bioconjugation candidates. Both PEGs were purchased from Laysan Bio company. Both compounds were very effective in the bioconjugation reaction and complete PEGylation of CA was observed in most cases.

Reaction of the primary amino group of the enzyme with a PEG succinimide ester results in formation of an amide. PEG-NHS esters have been extensively used in PEGylating enzymes, as they are extremely reactive towards the amino groups on the CA enzyme.

The bioconjugation reaction of PEG-NHS esters with CA was found to be very rapid. The reaction was usually complete in less than an hour depending on the pH, temperature, and ratio of PEG NHS ester to protein used. Conditions for the bioconjugation reaction were carefully chosen to minimize hydrolysis of the PEG-succinimide ester. It was found that hydrolysis was an issue, particularly for esters with short chain lengths between the reactive carboxyl and the last PEG ether group (Table 4.3).

TABLE 4.3
Hydrolysis Half-lives of PEG NHS Ester at pH 8, 25° C.

| | | Half-life |
|---|---|---|
| PEG NHS Ester | Ester (Symbol) | (minutes) |
| PEG-O—CH₂CH₂CH₂CH₂—CO₂- | Succinimidyl Valerate | 33.6 |
| NHS | (SVA) | |
| PEG-O—CO₂-NHS | Succinimidyl Carbonate | 20.4 |
| | (SC) | |
| PEG-O—CH₂CH₂—CO₂-NHS | Succinimidyl Propionate | 16.5 |
| | (SPA) | |
| PEG-O—CH₂—CO₂-NHS | Succinimidyl | 0.75 |
| | Carboxymethyl (SCM) | |

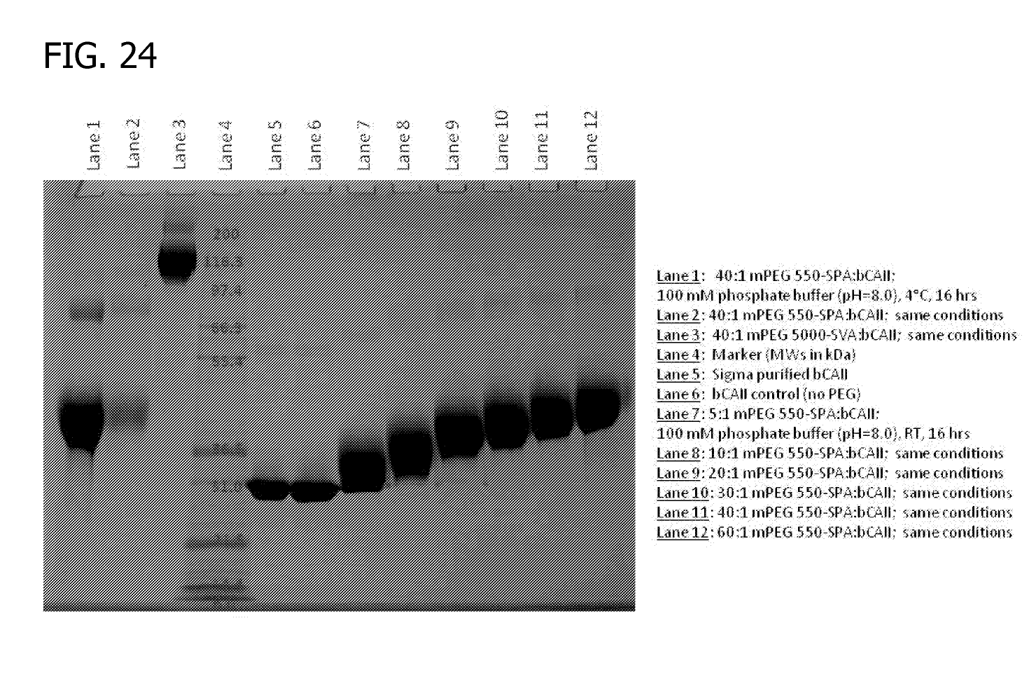

A series of reactions were performed using PEG succinimidyl propionate ester (mPEG 550 Da-SPA) at varying PEG:CA ratios (from 5:1 to 60:1) at room temperature in pH 8.0 phosphate buffer (100 mM) for 16 hours. The SDS PAGE results of that series are shown in FIG. 24 (Lanes 7-12). As seen in this figure, increasing the PEG molar ratio caused clear shifts in the protein band to higher molecular weights and indicated a greater extent of PEGylation. At a 5:1 molar ratio of functionalized PEG to bCAII, the protein band was not completely resolvable from the parent protein band, and possibly indicated the presence of unPEGylated enzyme. At higher PEG ratios, however, the band was completely shifted, meaning that all of the enzymes had been PEGylated by at least one PEG chain.

Comparing these results with that of epoxides and the sulfonate esters indicated that the succinimides were several orders of magnitude more reactive. A reaction time study was done with mPEG-5000 Da-SVA and bCAII, and it was observed that the bioconjugation reaction was complete within an hour, depending on the ratio of PEG to enzyme and pH of the reaction.

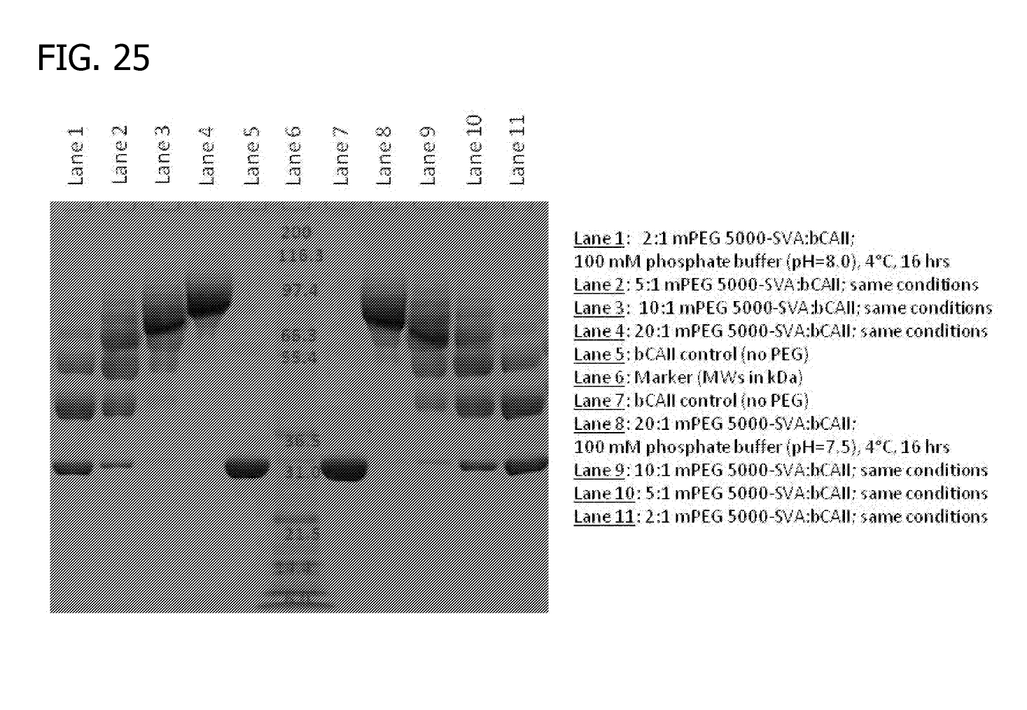

A series of reactions was set up using the methoxy PEG 500 Da-SVA (mPEG 5000 Da-SVA) at varying PEG:CA ratios (from 2:1 to 20:1) at 4° C. either in pH 8.0 or pH 7.5 phosphate buffer (100 mM) for 16 hours. The SDS PAGE results of that series are shown in FIG. 25. As seen in this figure, increasing the PEG molar ratio caused clear shifts in the protein band to higher molecular weights and indicated a higher extent of PEGylation. At a 5:1 molar ratio of functionalized PEG to bCAII, a faint band was still evident where the native protein appeared (at ˜31 kDa), which indicated the presence of some unPEGylated enzyme. This result was consistent with the work done with the mPEG 550 Da-SPA, which also indicated the presence of unmodified bCAII at 5:1 molar ratios of PEG to CA under these conditions.

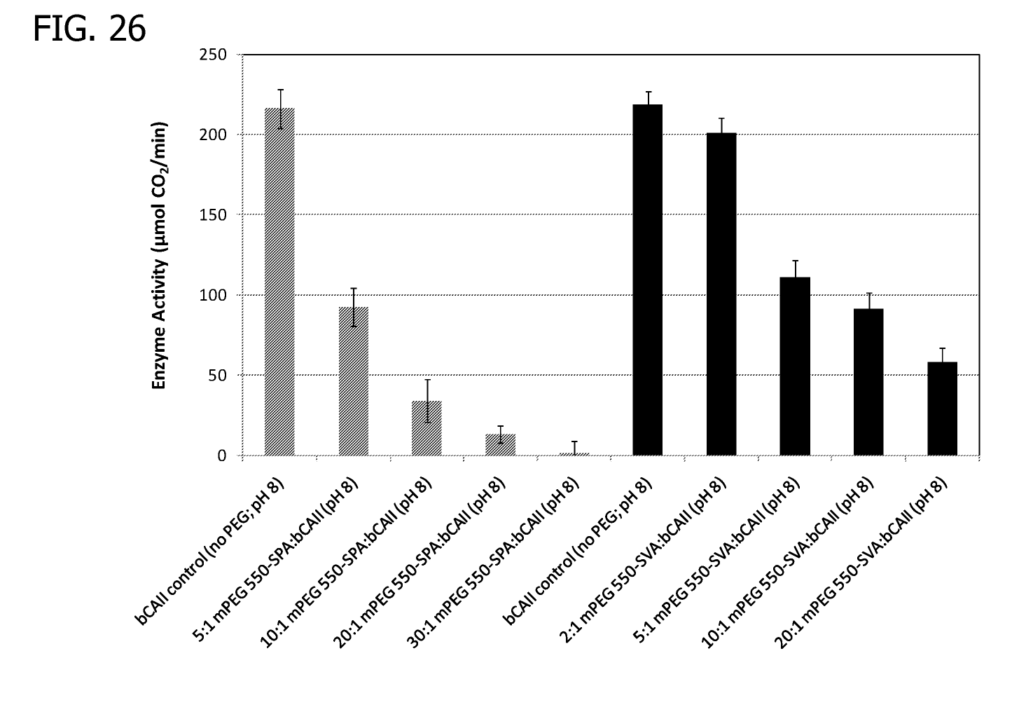

The enzymatic activity of PEGylated bCAII using both mPEG 550 Da-SPA and mPEG 5000 Da-SVA were obtained using the pH stat. Those results are shown in FIG. 26. The 2:1 mPEG 5000-SVA:CA samples retained most of their activity, but higher PEG ratios had a significant negative impact on enzyme activity. However, the higher mPEG 5000 Da-SVA ratio samples with no unmodified bCAII present still had some measurable activity, in contrast to the activity results using the shorter mPEG 550-SPA.

In our hands, PEGylation of bCAII with PEG NHS esters was very effective, but enzymatic activity after PEGylation as measured by the pH stat was minimal, especially in the cases of complete PEGylation of the enzyme. Because of the enzyme activity results, this class of chemistry was determined to be less than optimal for tethering bCAII into the micellar polymer.

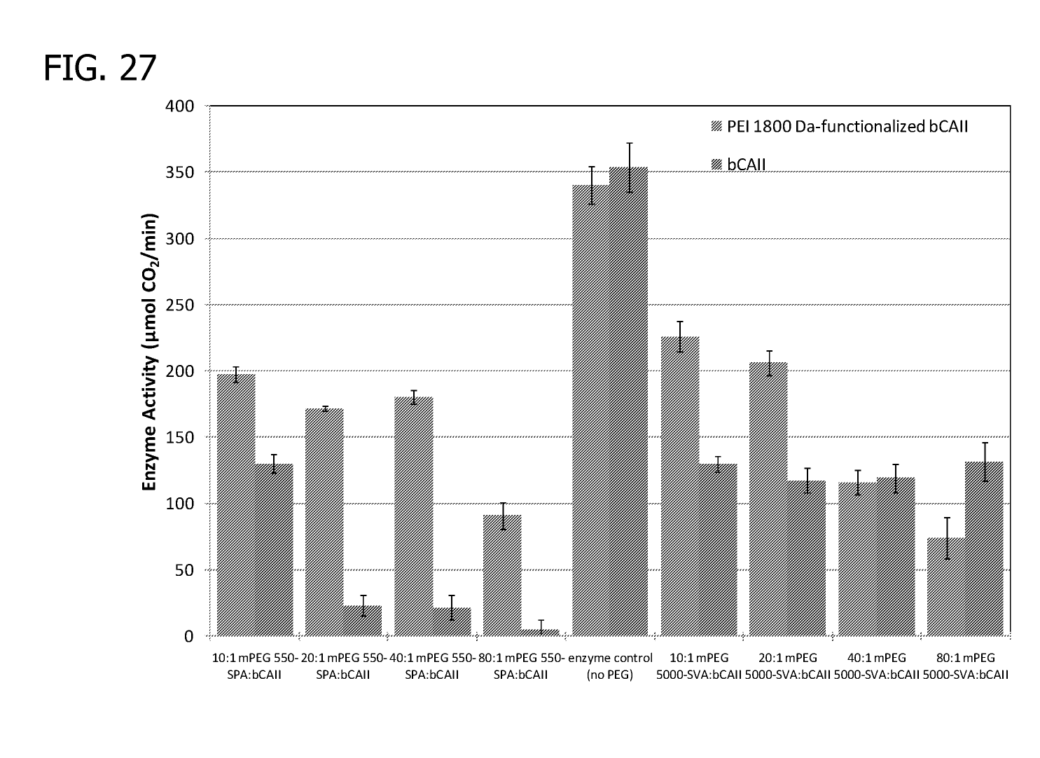

FIG. 27 compared the activity of PEG-succinimide conjugation at increasing molar ratios of mPEG 550 Da-succinimidyl propionate (SPA) and mPEG 5000 Da-succinimidyl valerate (SVA) using unmodified bCAII as well as PEI 1800 Da-functionalized bCAII. During the PEI functionalization of bCAII, the PEI 1800 Da was in 100-fold molar excess with a 60:1 ratio of EDC to bCAII. As seen in FIG. 27, the presence of PEI on the CA surface preserved some of the enzyme activity after bioconjugation. At higher ratios of PEG-succinimide to bCAII, however, the activities using PEI-functionalized bCAII became closer to those seen using unmodified bCAII.

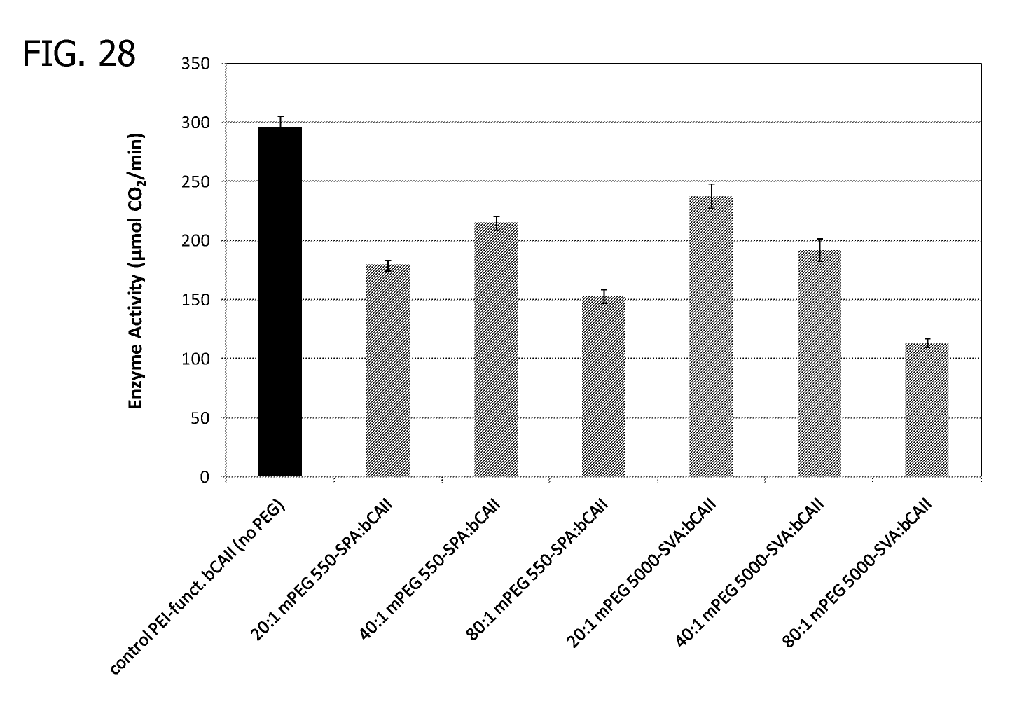

FIG. 27 used a low molecular weight PEI (1800 Da) at a low molar excess relative to the EDC used (100:60:1 PEI:EDC:bCAII). After lyophilization, this PEI-functionalized bCAII was mostly protein by weight (˜75%). By changing the molecular weight and/or the molar excess of the polyamine used, it was possible to introduce more primary amino functionalities to the bCAII surface, which provided better preservation of enzyme activity after reaction with PEG-succinimides. For instance, PEI functionalization with a higher molar excess (500:60:1 PEI 1800 Da:EDC:bCAII) gave a PEI-functionalized bCAII that was only 40% protein by weight after lyophilization, which indicated a higher surface coverage of PEI chains. As seen in FIG. 28, bioconjugation between this PEI-functionalized bCAII and PEG-succinimides at various ratios resulted in higher enzymatic activity than the corresponding reaction with bCAII with a lower polyamine content, particularly at higher molar excesses of PEG-succinimides.

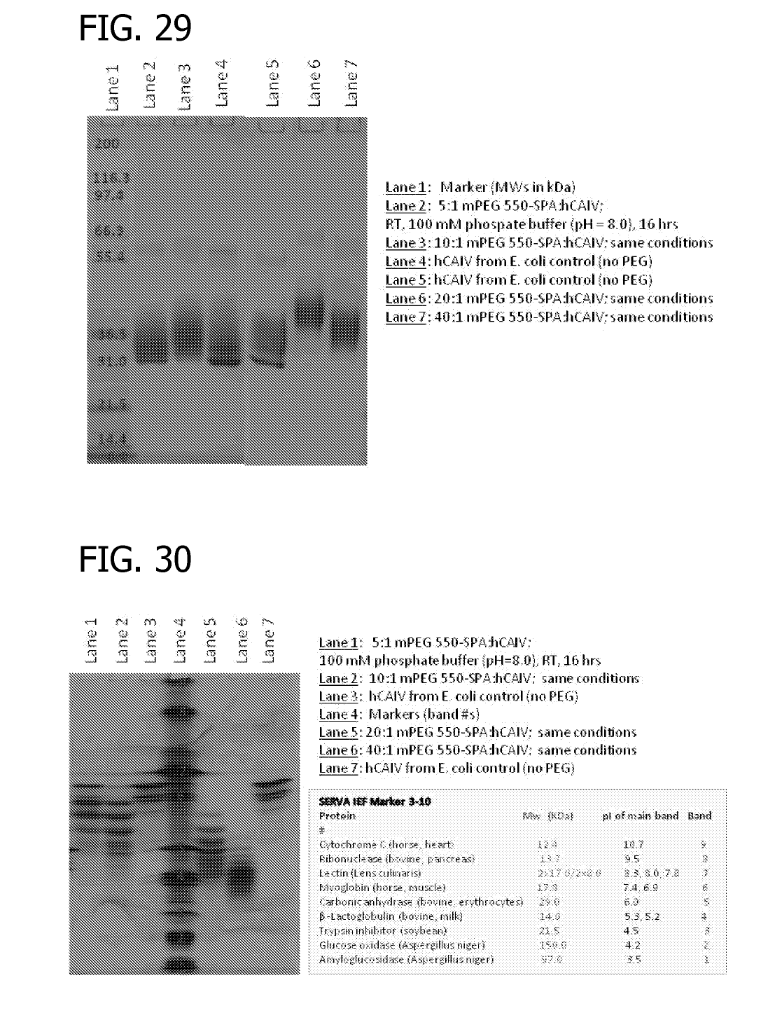

The above bioconjugation experiments using mPEG 500 Da-SPA were repeated using a different CA enzyme (hCAIV) to determine if the activity loss upon conjugation was specific to the isoform used. The SDS PAGE results using hCAIV are highlighted in FIG. 29. Because the native protein band was so broad for hCAIV (Lanes 4 and 5), it was difficult to ascertain if any unmodified enzyme remained in these reactions based on the band shift alone. Due to the difficulty of determining the amount of unmodified enzyme present via SDS PAGE analysis, isoelectric focusing (IEF) gels were also performed using these samples. Those results are shown in FIG. 30. This gel indicated that the 5:1 PEG to hCAIV sample still had some unreacted CA present (Lane 1), but higher ratios did not.

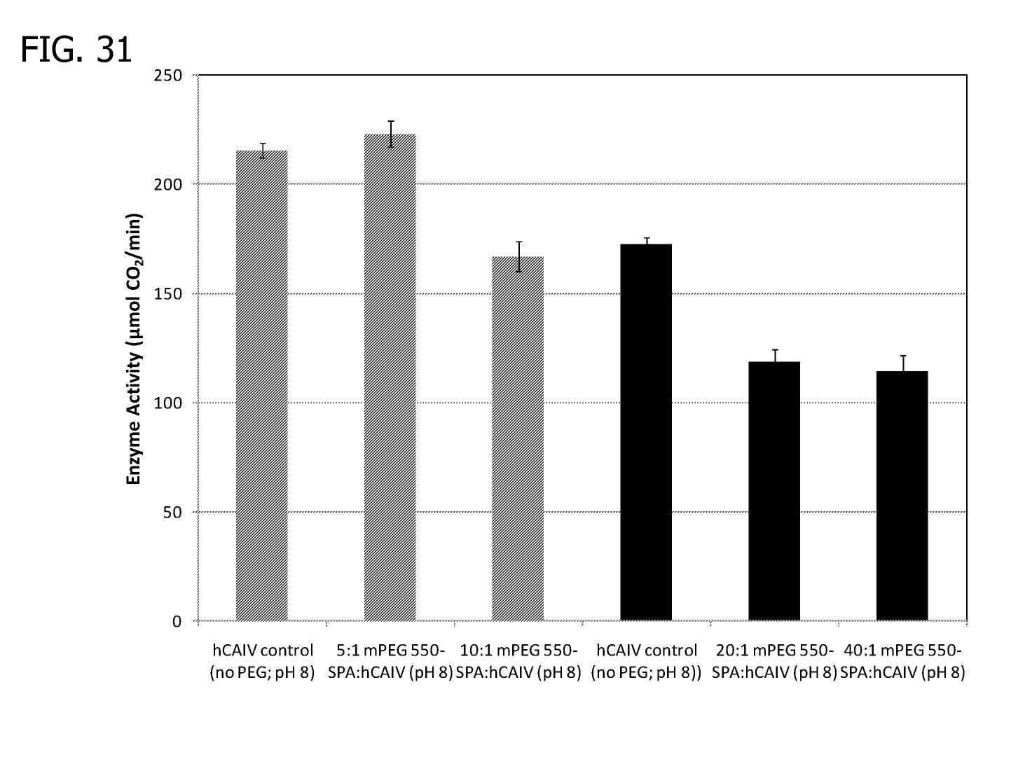

The enzymatic activity of these samples was determined using the pH stat, as shown in FIG. 31. In contrast to the results with bCAII, (FIG. 26), PEGylation of hCAIV using mPEG 550 Da-SPA did not result in a complete loss of activity. In fact, at the highest PEG ratios where all of the enzyme has reacted with at least one PEG chain, >50% of the activity still remained. As such, the succinimide conjugation chemistry was considered a potential route for tethering CA into the micellar polymers with isoforms other than bCAII.

### Example 4(d)

### PEG Aldehydes

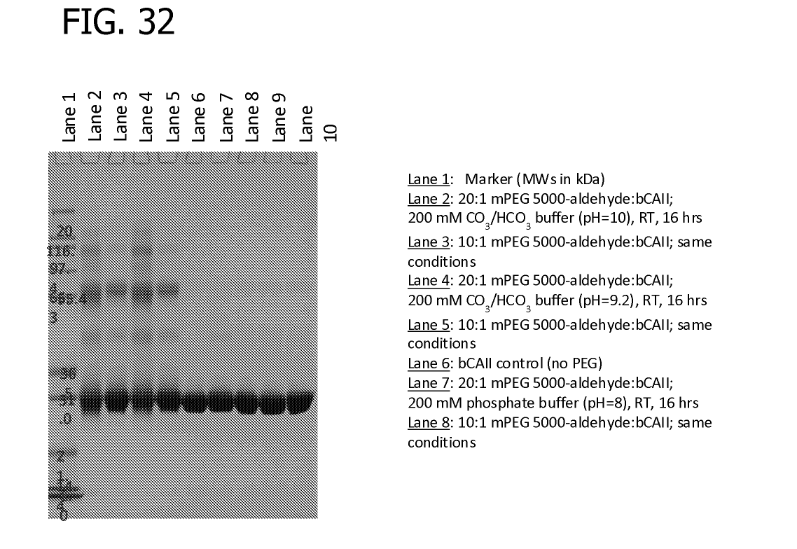

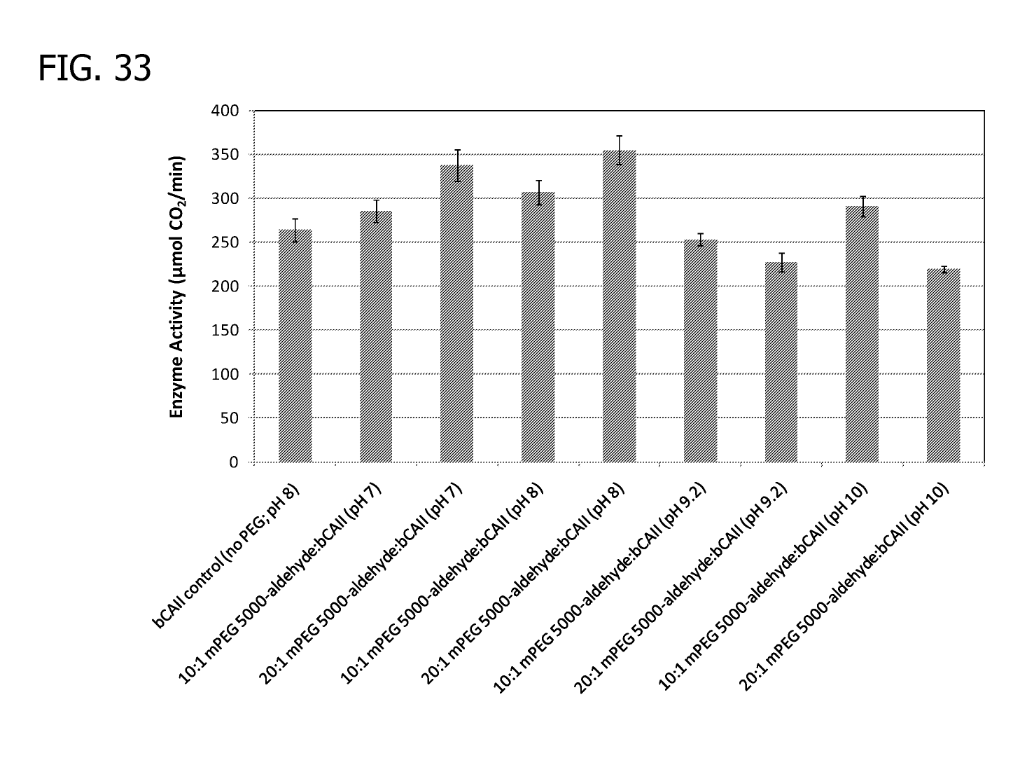

FIG. 32 shows the SDS PAGE results of PEG aldehyde bioconjugation with bCAII at room temperature as a function of pH. Below pH of 9.2, little to no reaction occurred. At higher pH, some reaction occurred, particularly at a 20:1 molar ratio of PEG-aldehyde to bCAII. The majority of the enzyme present in the mixture, however, was still unmodified. Thus, the aldehyde bioconjugation reaction was not very efficient under these conditions. The enzymatic activity of these samples are shown in FIG. 33. While these activities are high, little of the bCAII reacted with PEG aldehydes under these conditions. Thus these results did not provide a definite indication of the activities of PEGylated bCAII via aldehyde conjugation.

### Example 4(e)

### PEG Carbonyl Imidazoles

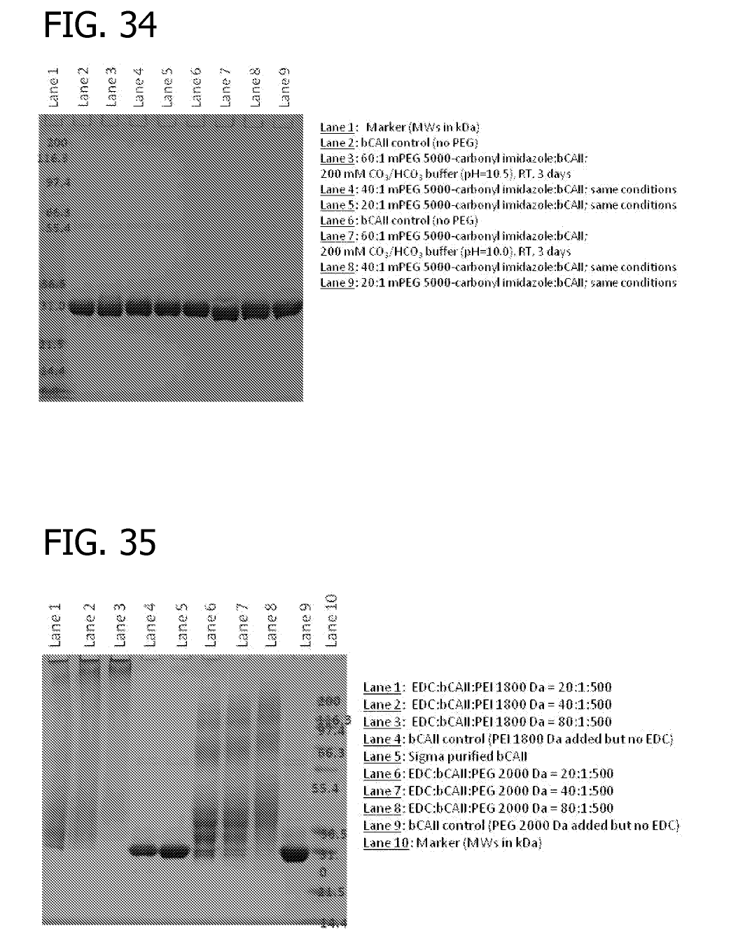

PEG carbonyl imidazole react with the primary amino groups of proteins to form a carbamate. FIG. 34 shows the SDS PAGE results of several bioconjugation experiments using PEG 5000 Da-carbonyl imidazole and bCAII. No reaction between this type of functionalized PEG and bCAII was observed.

### Example 5

### Functionalization of CA to Increase its Size

In an effort to minimize enzyme leaching from the micellar polymer matrix, several strategies were developed to increase the effective size of CA by conjugating it to various bulky substituents such as hydrophilic polymers, other proteins, or inert supports such as nanobeads.

### Example 5(a)

### Attachment of Hydrophilic Polymers to CA Surface

A myriad of hydrophilic polymers can be attached to the surface of CA via traditional bioconjugation techniques. Such hydrophilic polymers include polyamines such as polyethyleneimine (PEI), polyalkylene glycols such as polyethylene glycol (PEG), polyelectrolytes (e.g., polylysine, polyarginine, polyacrylic acid), carbohydrates (e.g., cellulose, chitosan, dextran, alginate), and poly(vinyl alcohol). A variety of polymer molecular weights and architectures can be used in this approach to tailor the resulting size of the modified CA.

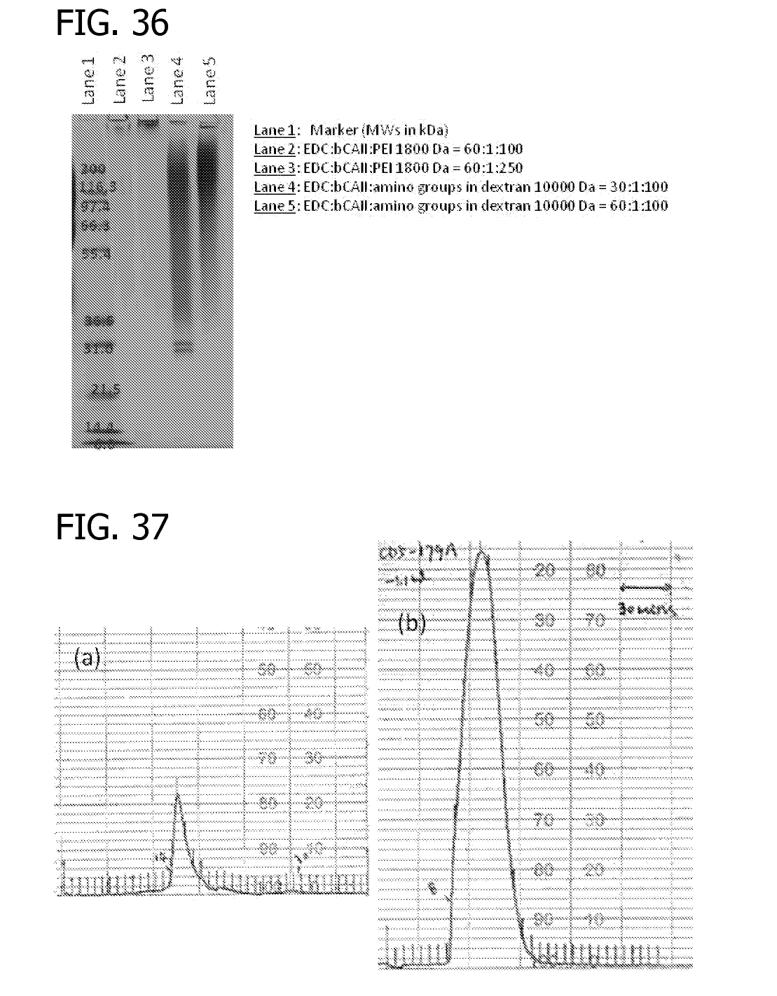

For example, carbodiimide (such as 1-ethyl-3-(3-dimethylaminopropyl)carbodiimide or EDC) conjugation chemistry was employed with hydrophilic amine-containing polymers to couple to the carboxylic acid groups of CA. FIG. 35 and FIG. 36 show the sodium dodecyl sulfate polyacrylamide gel electrophoresis (SDS-PAGE) results of a series of reactions with differing ratios of EDC:CA using polyethyleneimine (PEI) 1800 Da, polyethylene glycol (PEG) diamine 2000 Da, or amino-dextran 10000 Da in excess. As seen in the gel results, the increased EDC ratios did not improve the extent of functionalization of bCAII. At ratios of EDC:CA of 40:1 and higher, it appeared that all of the bCAII had been functionalized with at least one hydrophilic polymer chain. Once functionalized with hydrophilic polymers, these modified CA enzymes shifted to significantly higher equivalent molecular weights, which indicated an increase in their hydrodynamic diameters.

The shift to increased molecular weights seen in the SDS PAGE results of FIG. 35 and FIG. 36 with bCAII was verified using a preparatory SEC column (Sephacryl® 300-HR, molecular weight range of 10-1500 kDa for globular proteins). The chromatograph of bCAII functionalized with amino-dextran 10000 Da at an EDC:CA ratio of 60:1 is shown in FIG. 37(b), as compared to the native enzyme in FIG. 37(a). Upon functionalization with amino-dextran, bCAII shifted to higher molecular weights, with the largest functionalized enzyme eluting at the upper molecular weight limit of the column (1.5 million Da protein equivalent).

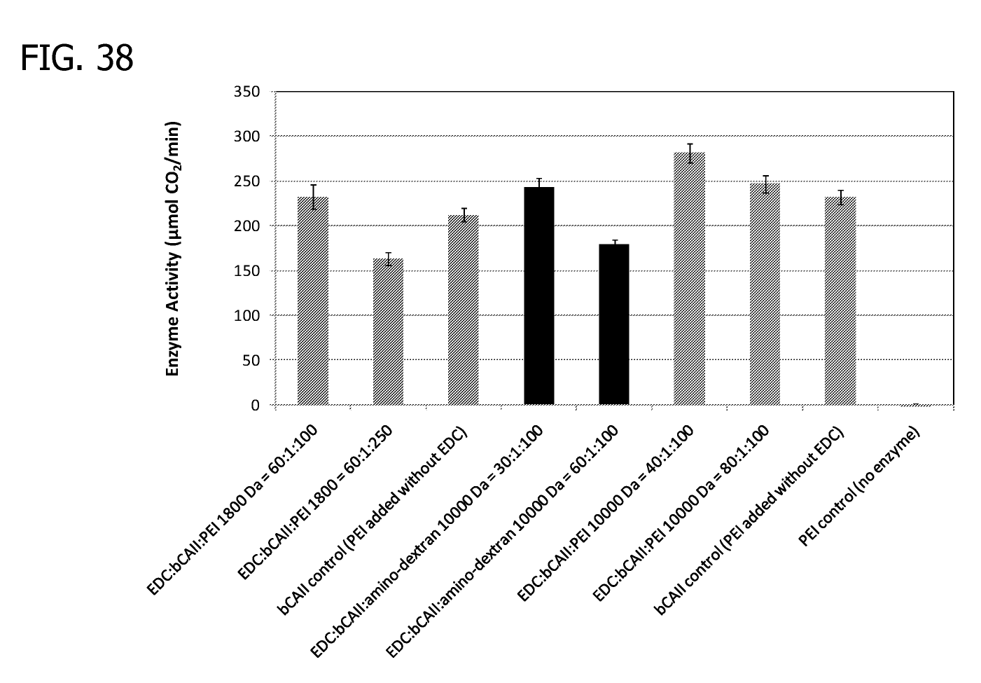

The CO₂ hydration activities of bCAII samples functionalized with various hydrophilic polymers were assayed using the pH stat, as shown in FIG. 38. As seen in this figure, functionalization of bCAII with polymers such as PEI and amino-dextran did not significantly impact the enzyme's activity at any of the EDC:CA ratios investigated. Furthermore, the hydrophilic polymers themselves did not catalyze CO₂ hydration and thus did not demonstrate any measurable activity on the pH stat.

### Example 6

### Cross Linking of CA

Since the polysiloxane-based micellar polymer mixtures have both silicon hydride (Si—H) and silanol (Si—OH) bonds, these polymers can be crosslinked into rubbery solids using several mechanisms. Thus, carbonic anhydrase can be functionalized with a variety of functional groups that can then react with the micellar polymer during crosslinking, including silanols (Si—OH), methoxysilanes (Si—OCH₃), and vinyl or allyl groups (CH₂═CH₂—R).

One procedure used carbodiimide chemistry (such as 1-ethyl-3-(3-dimethylaminopropyl)carbodiimide or EDC) to couple hydrophilic amine-silanols such as N-(2-aminoethyl)-3-aminopropylsilanetriol (AEAPS) or 3-aminopropylsilanetriol (APS) to the carboxylic acid groups of carbonic anhydrase.

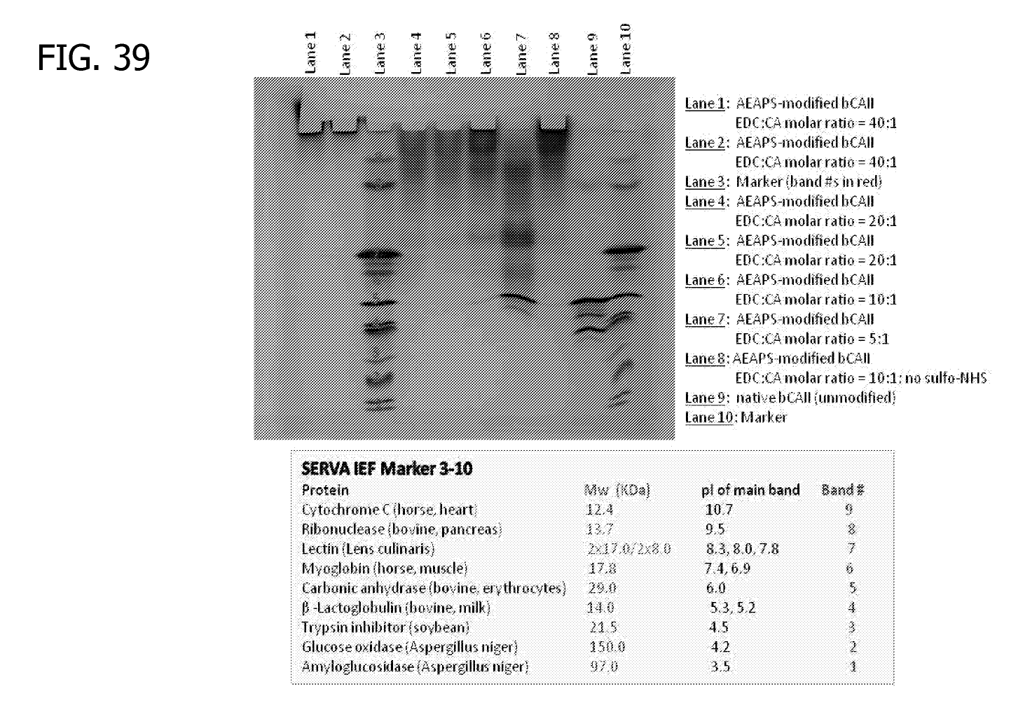

Changing the EDC:CA molar ratio changed the degree of functionality, which was analyzed using isoelectric focusing (IEF) gel electrophoresis. FIG. 39 shows the IEF gel results of functionalizing bCAII with AEAPS at differing EDC:CA molar ratios. As seen in this figure, at EDC:CA ratios of greater than 5:1, no unmodified enzyme was left in the reaction mixture. Increasing the EDC:CA ratio shifted the functionalized enzyme to higher isolelectric points (pI values), which indicated a higher degree of functionality.

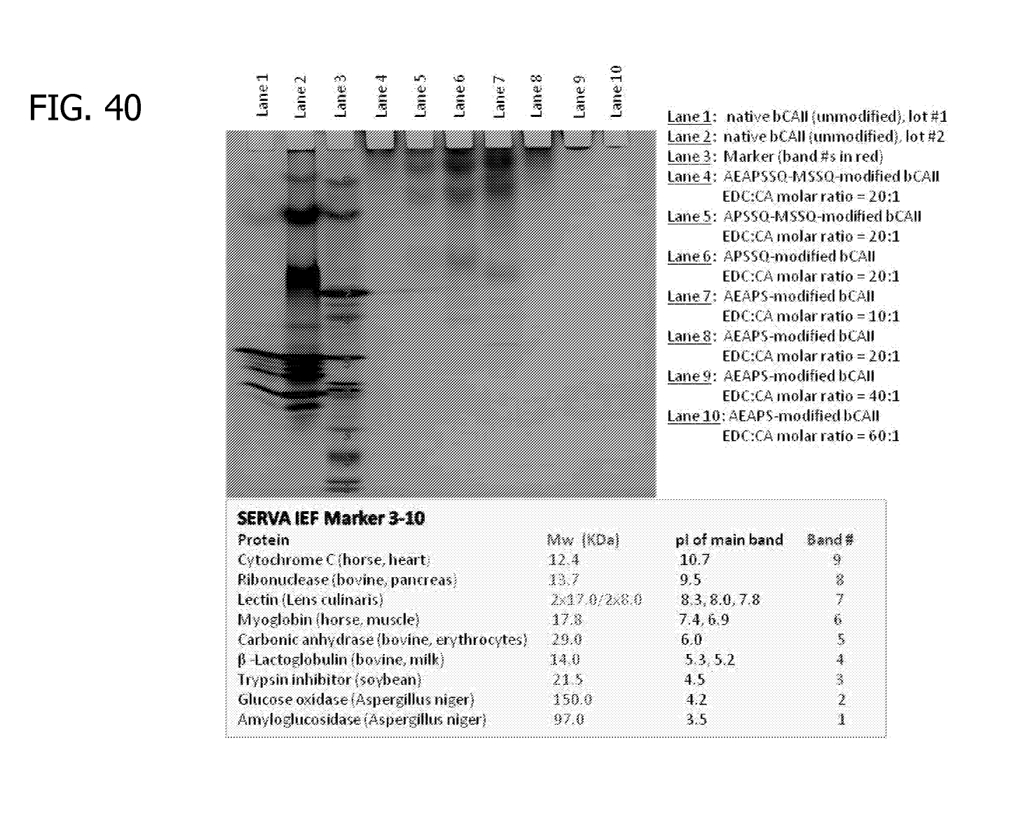

Alternately, CA was functionalized with oligomeric hydrophilic amine-silanols known as silsesquioxanes that have more silanol groups available for reacting with the micellar polymer during crosslinking IEF gels on bCAII functionalized with these polymeric amine-containing silsesquioxanes showed comparable results to the small-molecule silanols, such as AEAPS (FIG. 40).

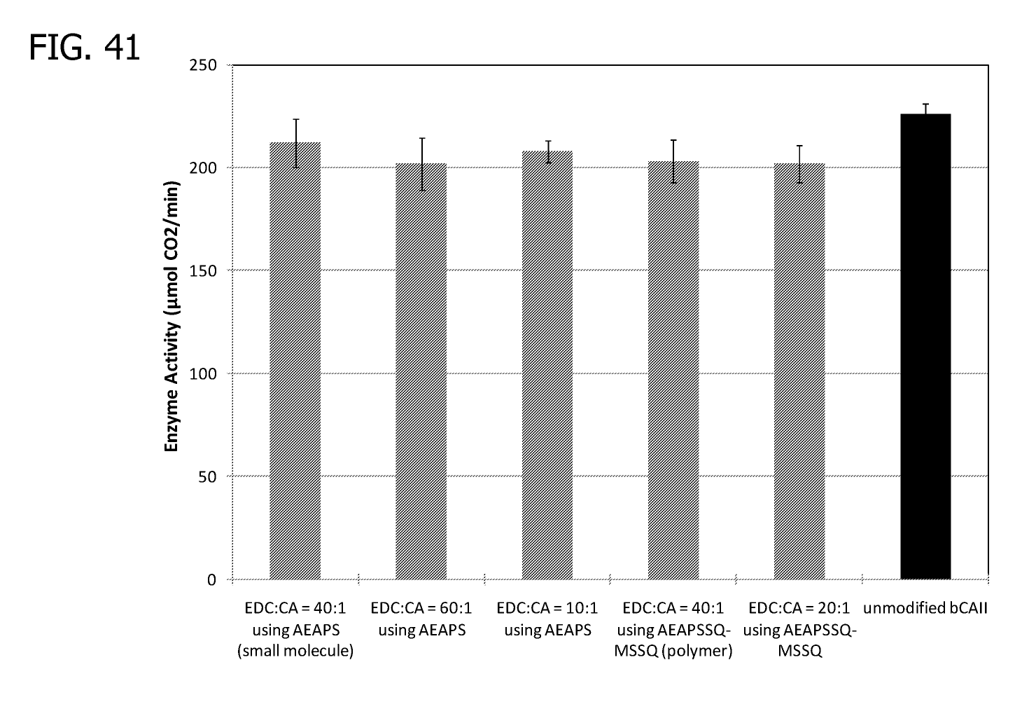

Activity comparisons of modified enzymes after undergoing EDC coupling of various amine-silanol modifiers with different degrees of functionality are shown in FIG. 41. As seen in this figure, modifying bCAII with both the small molecule amine-silanols (AEAPS) and the copolymer AEAPSSQ-MSSQ with differing EDC:CA ratios resulted in no appreciable loss in enzyme activity.

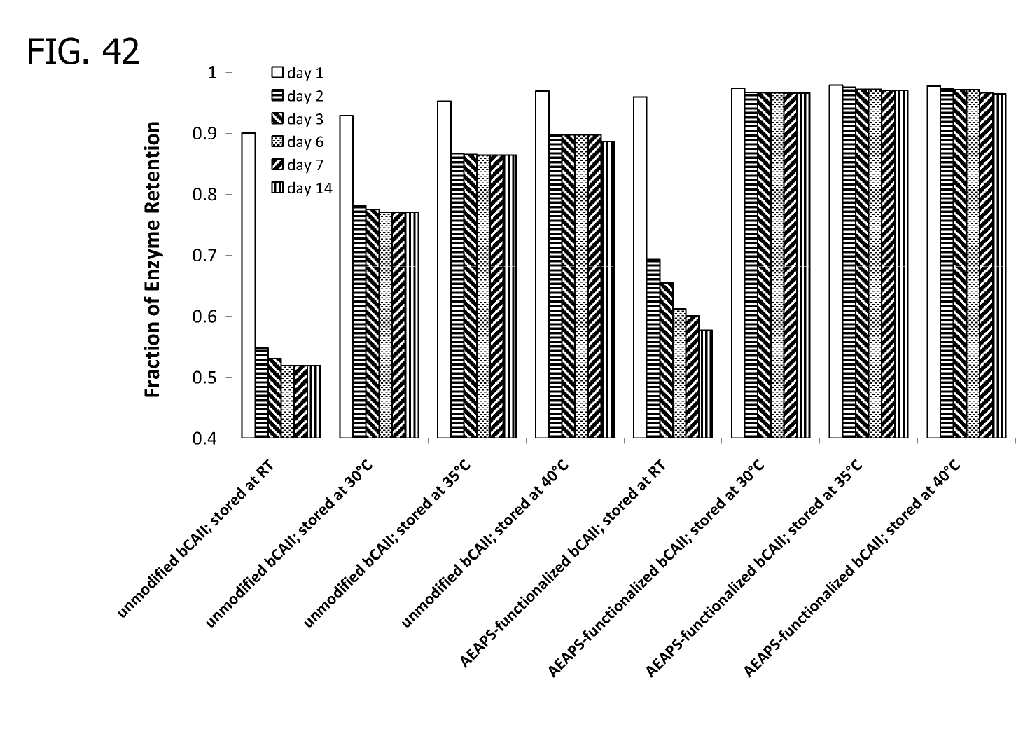

Initial leaching studies of modified vs. unmodified bCAII immobilized in a micellar polymer coating on functionalized ceramic indicated an improvement in physical enzyme retention at all storage temperatures investigated, as shown in FIG. 42.

### Example 7

### Surface Functionalization of Support Material to Improve Adhesion

Initial experiments in coating various supports showed that the immobilization matrix did not adhere well to the underlying substrate and would easily slough off under agitation. As such, various surface functionalization chemistries were identified to modify common column packing materials (e.g., ceramics, graphite, stainless steel, plastics) with groups capable of coupling to the micellar polymer coating during crosslinking

### Example 7(a)

### Ceramic/Graphite

Scheme 15 shows the experimental procedure for functionalizing ceramic or graphite-based support materials. After etching to introduce surface hydroxyl groups, isocyanate groups were introduced and then coupled to disilanol-terminated PDMS. The resulting silanol groups were reacted with the micellar polymer layer during crosslinking to covalently attach the immobilization matrix to the support.

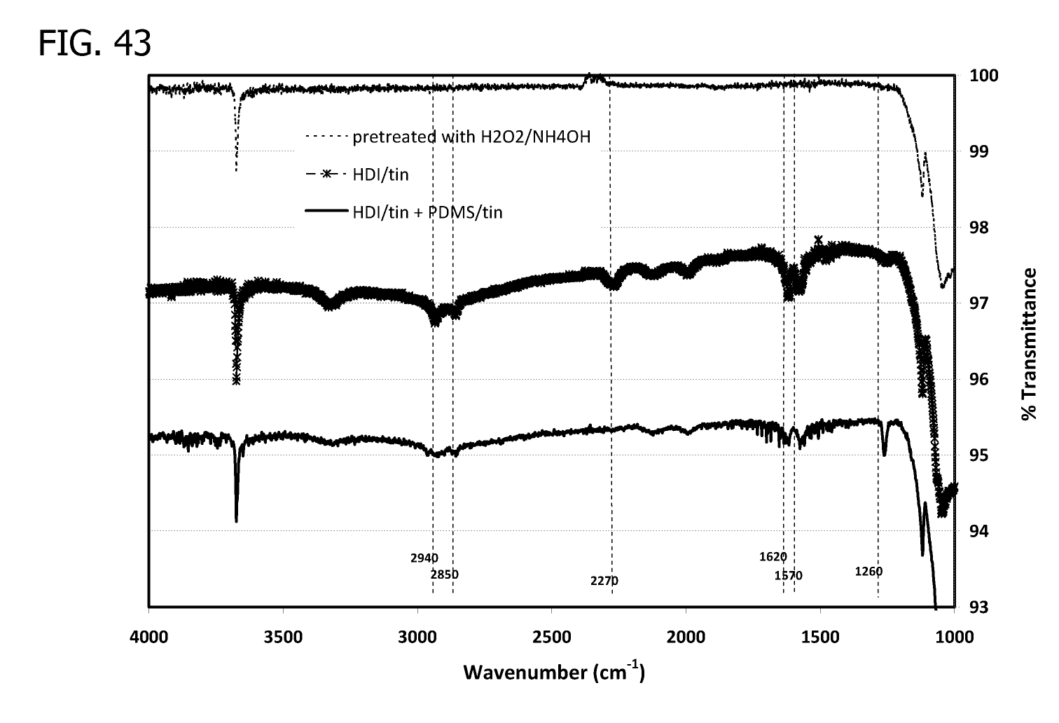

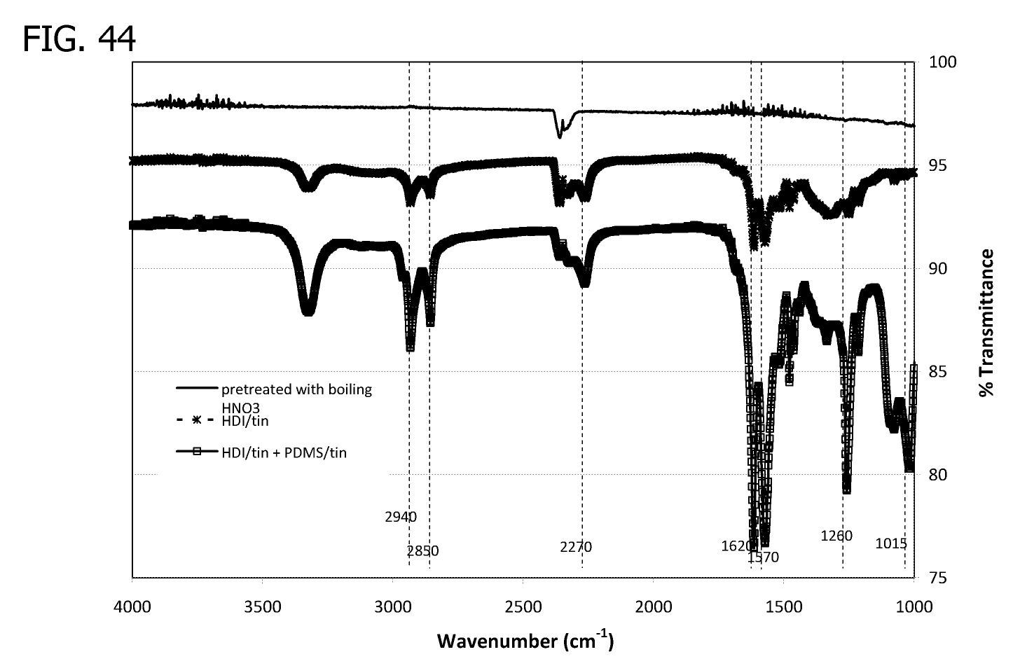

Surface functionalization of the support materials was confirmed using attenuated total reflectance Fourier transform infrared spectroscopy (ATR FTIR). FIG. 43 shows the FTIR spectra results for functionalized ceramic, while FIG. 44 shows the same functionalization of graphite. The top spectrum in both figures (blue line) shows the support material after a pretreatment etching step. The second spectrum (red line) is the same material after functionalizing it with hexamethylene diisocyanate at 60° C. catalyzed by a tin catalyst such as dibutyldilauryltin. The presence of isocyanate groups on the surface was confirmed by the isocyanate (N═C═O) stretching peak located at 2270 cm⁻¹ as well as the C═O stretching peaks at 1620 and 1570 cm⁻¹ and the CH₂ peaks at 2940 and 2850 cm⁻¹. The same reaction without the tin catalyst, even at the higher temperature of 90° C., did not show any appreciable isocyanate groups on the surface. The third spectrum (green line) was the same isocyanate-functionalized support after a second functionalization step with disilanol-terminated PDMS (MW=4200 g/mol), again catalyzed by an organometallic tin compound. This spectrum shows an additional peak at 1260 cm⁻¹, corresponding to the Si—CH₃ peaks of the PDMS grafted on the surface. In FIG. 44, the peak in the green spectrum at 1015 cm⁻¹ was due to the presence of Si—O—Si bonds from PDMS. The presence of the isocyanate peaks in the green spectra indicated an incomplete surface coverage of PDMS. Since the reaction between isocyanates and silanols was catalyzed by the same tin catalysts used during the crosslinking reaction, it may be sufficient to functionalize the surface with isocyanate groups only. These groups could then react with the silanol groups in the micellar polymer mixture during crosslinking to covalently attach the micellar polymer film to the support.

### Example 7(b)

### Steel

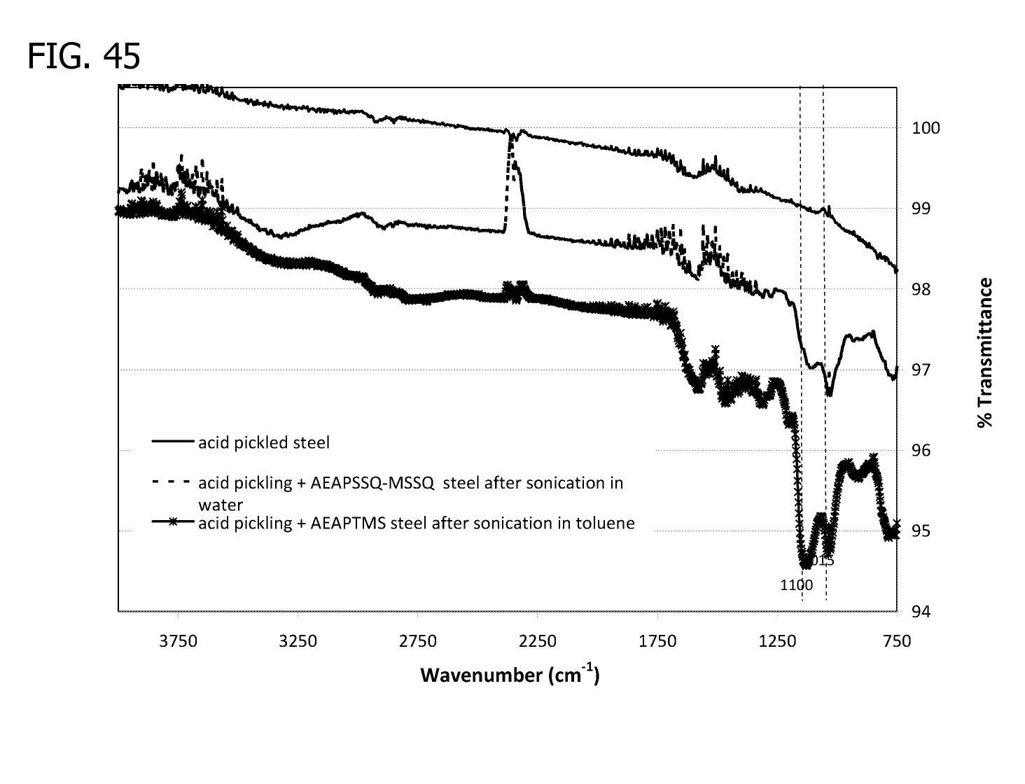

After an acid pickling or electrochemical reduction step to remove the surface oxide layer, stainless steel was functionalized with a variety of amine or sulfur-containing compounds that coordinated with the exposed metal atoms of the freshly etched steel surface. (Ruan, C. M.; Bayer, T.; Meth, S.; Sukenik, C. M. Thin Solid Films 2002, 419, 95-104) FIG. 45 shows the FTIR spectra of stainless steel functionalized with two different amine compounds. Aminoethylaminopropyltrimethoxysilane (AEAPTMS) functionalized steel with methoxysilane groups that can couple to the silanol groups of the micellar polymer mixture during crosslinking. Aminoethylaminopropylsilsesquioxane-methylsilsesquioxane (AEAPSSQ-MSSQ) copolymer functionalized steel with silanol groups that react with silicon hydride groups during crosslinking. The peaks at 1100 cm⁻¹ and 1015 cm⁻¹ are due to the Si—O—Si bonds present on the surface and indicated surface functionalization by the amine-siloxanes. These peaks were still present after extensive sonication in solvents in which they were readily soluble and indicated that both AEAPTMS and AEAPSSQ-MSSQ were covalently attached to the steel surface rather than physically adsorbed.

### Example 8

### Synthesis of Allyl-PEG-Epoxide

A 3 neck round bottom flask was charged with allyl PEG-OH 500 Da (˜50 g, 0.10 mol) and the PEG was chilled to 0° C. using an ice bath. Note: The PEG does solidify if it gets too cold. Epichlorohydrin (32 ml, 0.32 mol) was charged to an addition funnel and added to the reaction mixture slowly. After this charge, sodium hydroxide (14 g, 0.35 mol) was added portion wise three times. Approximately 1 hour after the charge of sodium hydroxide was complete, the bath was removed and the reaction mixture was allowed to warm to room temperature and stirred for 5 hours.

Dichloromethane (200 ml) was added and the reaction mixture was filtered through a plug of silica gel. The filtrate was evaporated under reduced pressure to a reduced volume. Pyridine (25 mL) was added and the mixture was stirred at room temperature for 1 hour. The product mixture was then transferred to a reparatory funnel and washed with 5% aqueous HCl solution (2×100 mL), water (1×100 mL), and finally with brine (1×100 mL). The organic phase containing the product was then dried over anhydrous magnesium sulfate, filtered, and evaporated under reduced pressure to give 49 g of the product as an oil in 89% yield. ¹H NMR (CDCl₃) δ 5.89, (m, 1H), 5.22 (dd, J=8.35, 13.7 Hz, 2H), 4.01, (d, J=4.52 Hz, 2H), 3.77, (dd, J=6.9, 9.30 Hz), 3.64, (m, 42-44H), 3.42, (m, 1H), 3.28 (m, 1H), 2.60 (m, 1H), 2.29 (m, 1H).

### Example 9

### Synthesis of Allyl-PEG-Tresylate

An oven-dried 250 mL 3 neck round bottom flask equipped with a thermocouple was charged with anhydrous THF (30 mL). Allyl-PEG-OH 500 Da (10 g, 0.02 mol) was added, and the clear solution was cooled to −10° C. with an ice and salt bath. Tresyl chloride (4.38 g, 0.02 mol) was added. To the cold solution was added powdered sodium hydroxide (3 g, 0.08 mol) in 3 portions over 45 minutes. After addition of the base was complete, the reaction mixture was stirred at 0-8° C. for 2 hours. The temperature of the reaction was maintained at 0° C. or lower. The reaction mixture was then dissolved with 200 mL of diethyl ether, and the salts were removed by vacuum filtration. The filtrate was evaporated under reduced pressure to give 12 g of crude allyl PEG-500. The product was purified by filtration through a plug of silica gel (eluent 30% ethyl acetate/hexanes (400 mL) then with 400 ml of 90% chloroform methanol). The filtrate was evaporated under reduced pressure to give 7 g of desired product. ¹H NMR (CDCl₃) 6 (m, 1H), 5.21 (dd, J=13.80, 38.75, 2H), 4.21 (m, 2H), 3.99 (m, 2H), 3.58 (m, 44H).

### Example 10

### Grafting Reaction of Allyl-PEG-Monomethyl Ether and Allyl Glycidyl Ether onto PHMS

Poly(hydrogenmethylsiloxane) (PHMS, MW_{avg}=2250 g/mol, 35 Si—H groups; 70 mL), allyl poly(ethylene glycol) monomethyl ether (PEG, MW_{avg}=500 g/mol; 75 mL), allyl glycidyl ether (AGE; 15 mL), and 400 mL dry toluene were added to a 3-neck 1 L round bottom flask equipped with teflon stir bar. The round bottom flask was fitted with a thermostat and heating mantle, a condenser with bubbler, and a nitrogen line. This setup was then purged with nitrogen for 20 minutes. Afterwards, 1 mL of 5 mM hexachloroplatinic acid (H₂PtCl₆) in isopropanol solution was injected into the reaction mixture via a gas-tight syringe. This reaction mixture was then slowly heated to 100° C. and allowed to react for 2 days under nitrogen while stirring. The reaction was then cooled to room temperature and stirred over activated carbon to remove the platinum catalyst. The carbon was then removed via filtration, and the toluene was removed under reduced pressure to yield a viscous clear liquid poly(hydrogenmethylsiloxane)-graft-poly(ethylene glycol)/glycidyl ether (PHMS-g-PEG/GE). The average molecular weight, PEG content, and epoxide (glycidyl ether) content can be determined via ¹H NMR spectroscopy in deuterated chloroform. By comparing the epoxide peaks (2.6 ppm, 2.8 ppm, and 3.1 ppm; 1H each per epoxide unit), methoxy peak of PEG (3.4 ppm; 3H per PEG chain), and silicon hydride peak (4.7 ppm) and knowing that each PHMS chain has 35 silicon hydride groups initially, it was determined that this graft copolymer has an average molecular weight of 7000 g/mol, 67 wt. % PEG, and 2.6 epoxide moieties per PHMS chain. The PEG content and number of epoxide moieties can be manipulated by changing the ratios of starting materials.

### Example 11

### Grafting Reaction of Allyl-PEG-Monomethyl Ether and Functionalized Allyl-PEG onto PHMS Using Speier's Catalyst

Functional groups that can couple to carbonic anhydrase can be incorporated into the micellar polymer by grafting allyl-PEGs that have been previously functionalized with these reactive moieties. For example, the synthesis below describes in detail the addition of a tosylate group into the micellar polymer.

Poly(hydrogenmethylsiloxane) (PHMS, MW_{avg}=2250 g/mol, 35 Si—H groups; 25 mL), allyl poly(ethylene glycol) monomethyl ether (PEG, MW_{avg}=500 g/mol; 20 mL), allyl-PEG-tosylate (MW_{avg}=700 g/mol; 9 mL), and 120 mL dry toluene were added to a 3-neck 500 mL round bottom flask equipped with teflon stir bar. The round bottom flask was fitted with a thermostat and heating mantle, a condenser with bubbler, and a nitrogen line. This setup was then purged with nitrogen for 20 minutes. Afterwards, 0.4 mL of 5 mM hexachloroplatinic acid (H₂PtCl₆) in isopropanol solution was injected into the reaction mixture via a gas-tight syringe. This reaction mixture was then slowly heated to 100° C. and allowed to react for 2 days under nitrogen while stirring. The reaction was then cooled to room temperature and stirred over activated carbon to remove the platinum catalyst. The carbon was then removed via filtration, and the toluene was removed under reduced pressure to yield a viscous clear liquid poly(hydrogenmethylsiloxane)-graft-poly(ethylene glycol)/poly(ethylene glycol)-tosylate (PHMS-g-PEG/PEG-tosylate). The average molecular weight, PEG content, and tosylate content can be determined via ¹H NMR spectroscopy in deuterated chloroform. By comparing the tosylate peaks [7.4 ppm and 7.8 ppm (2H each per tosylate group) and 2.3 ppm (3H per tosylate group)], methoxy peak of PEG (3.4 ppm; 3H per PEG chain), and silicon hydride peak (4.7 ppm) and knowing that each PHMS chain has 35 silicon hydride groups initially, it was determined that this graft copolymer has an average molecular weight of 7000 g/mol, 66 wt. % PEG, and 2.0 tosylate moieties per PHMS chain. The PEG content and number of functional moieties can be manipulated by changing the ratios of starting materials. Table 11.1 contains the details for additional synthesized micellar polymers containing functional moieties such as tosylate, nosylate, and epoxide groups at the end of hydrophilic PEG side chains.

TABLE 11.1
Synthetic details of additional grafting reactions of PHMS with allyl-PEG-methyl
ether and functionalized allyl PEGs

| | Allyl PEG | | | | | | | |
|---|---|---|---|---|---|---|---|---|
| | mono- | Functionalized | | H₂PtCl₆ | | | | # |
| PHMS | methyl | allyl PEG | Toluene | soln | Reaction | MWₜₒₜ | wt. % | functional |
| (mL) | ether (mL) | (mL) | (mL) | (mL) | temp° C. | (g/mol) | PEG | groups |
| 18 | 15 | 3.5; tosylate | 120 | 0.2 | 100 | 6300 | 62 | 1.3 |
| 50 | 40 | 20; tosylate | 240 | 0.7 | 100 | 7700 | 68 | 3.4 |
| 27.8 | 22.2 | 10; nosylate | 135 | 0.4 | 100 | 7900 | 69 | 2.0 |
| 25 | 20 | 8.4; nosylate | 110 | 0.35 | 100 | 7900 | 68 | 3.0 |
| 10 | 0 | 10; epoxide | 100 | 0.3 | ~115 | 9000 | 72 | 11.3 |
| | | | | | (reflux) | | | |

### Example 12

### Grafting Reaction of Allyl-PEG-Monomethyl Ether and/or Functionalized Allyl-PEG onto PHMS Using Platinum Oxide

Functional groups that can couple to carbonic anhydrase can be incorporated into the micellar polymer by grafting allyl-PEGs that have been previously functionalized with these reactive moieties. For example, the synthesis below describes in detail the addition of an epoxide group into the micellar polymer.

Poly(hydrogenmethylsiloxane) (PHMS, MW_{avg}=2250 g/mol, 35 Si—H groups; 10 mL), allyl poly(ethylene glycol) epoxide (epoxy-PEG, MW_{avg}=500 g/mol; 10 mL), 50 mL dry toluene, and 20 mg platinum oxide (PtO₂) were added to a 3-neck 500 mL round bottom flask equipped with teflon stir bar. The round bottom flask was fitted with a thermostat and heating mantle, a condenser with bubbler, and a nitrogen line. This setup was then purged with nitrogen for 20 minutes. This reaction mixture was then slowly heated to 70° C. and allowed to react overnight under nitrogen while stirring. The reaction was then cooled to room temperature and filtered to remove the platinum catalyst. The toluene was removed under reduced pressure to yield a viscous clear liquid poly(hydrogenmethylsiloxane)-graft-poly(ethylene glycol)-epoxide (PHMS-g-PEG-epoxide). The average molecular weight, PEG content, and epoxide content can be determined via ¹H NMR spectroscopy in deuterated chloroform. By comparing the epoxide peaks [3.1, 2.7, and 2.5 ppm (1H each peak per epoxide moiety) and silicon hydride peak (4.7 ppm) and knowing that each PHMS chain has 35 silicon hydride groups initially, it was determined that this graft copolymer has an average molecular weight of 4900 g/mol, 54 wt. % PEG, and 5.3 epoxide moieties per PHMS chain. The PEG content and number of functional moieties can be manipulated by changing the ratios of starting materials. Table 12.1 contains the details for additional synthesized PHMS-g-PEG micellar polymers using platinum oxide.

TABLE 12.1
Synthetic details of additional grafting reactions of PHMS with
allyl-PEG-methyl ether using platinum oxide

| | Allyl PEG | | | Reaction | | |
|---|---|---|---|---|---|---|
| PHMS | monomethyl | Toluene | PtO₂ | temperature | MWₜₒₜₐₗ | wt. % |
| (mL) | ether (mL) | (mL) | (mg) | (° C.) | (g/mol) | PEG |
| 10 | 40 | 100 | 18 | 80 | 13200 | 83 |
| 10 | 40 | 100 | 18 | 70 | 13700 | 84 |
| 10 | 10 | 50 | 23 | 50 | 5200 | 57 |
| 10 | 10 | 50 | 23 | 60 | 5600 | 60 |

### Example 13

### Grafting Reaction of Functional Groups onto PHMS-g-PEG Using Karstedt's Catalyst

Because the Karstedt's catalyst is a “hot” catalyst, it can be used at room temperature to react the remaining Si—H groups of PHMS-g-PEG micellar polymers with a variety of functional groups.

### Example 13(a)

### 4-Vinylbenzyl Chloride

Poly(hydrogenmethylsiloxane)-graft-poly(ethylene glycol) (PHMS-g-PEG, MW_{avg}=15500 g/mol, 85 wt. % PEG; 10 mL), 4-vinylbenzyl chloride (3.4 mL; 2-fold excess to Si—H groups) and 30 mL dry toluene were added to a 100 mL round bottom flask equipped with teflon stir bar. The round bottom flask was fitted with a nitrogen line and purge needle. This setup was then purged with nitrogen for 20 minutes. Afterwards, 0.05 mL of a platinum divinyltetramethyldisiloxane complex in xylenes (˜2% Pt; Karstedt's catalyst) was injected into the reaction mixture via a gas-tight syringe. This reaction mixture was then allowed to react for 3 hours under nitrogen while stirring. The reaction was then precipitated into 2 L of cold hexanes. The hexane supernatant was decanted, and the precipitate was dried in the vacuum oven to yield a viscous clear liquid poly(hydrogenmethylsiloxane)-graft-poly(ethylene glycol)/4-vinylbenzyl chloride (PHMS-g-PEG/4-benzyl chloride). The average molecular weight, PEG content, and benzyl chloride content can be determined via ¹H NMR spectroscopy in deuterated chloroform. By comparing the benzyl chloride peaks [4.6 ppm (2H each per vinylbenzyl group) and methoxy peak of PEG (3.4 ppm; 3H per PEG chain) as well as knowing that each PHMS chain has 35 silicon hydride groups initially, it was determined that this graft copolymer has an average molecular weight of 17000 g/mol, 85 wt. % PEG, and 8.5 benzylchloride moieties per PHMS chain.

### Example 13(b)

### Undecenoic Acid

Poly(hydrogenmethylsiloxane)-graft-poly(ethylene glycol) (PHMS-g-PEG, MW_{avg}=13800 g/mol, 84 wt. % PEG; 10 mL), undecenoic acid (4.4 g; 2-fold excess to Si—H groups) and 30 mL dry toluene were added to a 100 mL round bottom flask equipped with teflon stir bar. The round bottom flask was fitted with a nitrogen line and purge needle. This setup was then purged with nitrogen for 20 minutes. Afterwards, 0.05 mL of a platinum divinyltetramethyldisiloxane complex in xylenes (˜2% Pt; Karstedt's catalyst) was injected into the reaction mixture via a gas-tight syringe. This reaction mixture was then allowed to react for 3 hours under nitrogen while stirring. The reaction was then precipitated into 2 L of cold hexanes. The hexane supernatant was decanted, and the precipitate was dried in the vacuum oven to yield a viscous clear liquid poly(hydrogenmethylsiloxane)-graft-poly(ethylene glycol)/undecenoic acid (PHMS-g-PEG/undecanoic acid). The average molecular weight, PEG content, and carboxylic acid can be determined via ¹H NMR spectroscopy in deuterated chloroform. By comparing the (CH₂)₇ peak of undecanoic acid [1.3 ppm (14H per undecanoic acid group) and methoxy peak of PEG (3.4 ppm; 3H per PEG chain) as well as knowing that each PHMS chain has 35 silicon hydride groups initially, it was determined that this graft copolymer has an average molecular weight of 16000 g/mol, 84 wt. % PEG, and 12 undecanoic acid moieties per PHMS chain.

### Example 14

### Functionalization of Ceramic Packing for Improved Adhesion of Micellar Polymer film

Ceramic packing (250 mL) was etched first in either a 10% HF solution overnight (for silicate ceramics) or a 1:1:3 mixture of concentrated ammonium hydroxide: 30 wt. % hydrogen peroxide solution:water at 80° C. (for alumina ceramics) for 2 hours. The packing was rinsed thoroughly with water until the pH of the rinse was neutral, then with methanol, and dried in a vacuum oven at 40° C. overnight. The ceramic was then added to a 1 L round bottom flask with 200 mL dry toluene and 10 mL of hexamethylene diisocyanate (HDI) and 0.5 mL of bis(2-ethylhexanoate)tin catalyst. The flask was capped with a septum, purged with nitrogen for 10 minutes, and then put in a 60° C. shaker oven to react overnight. The packing was then rinsed with 250 mL toluene twice, 250 mL acetone twice, and dried in the vacuum oven at 40° C. for several hours. The ceramic was then put back in a 1 L round bottom flask with 200 mL dry toluene, 30 mL disilanol-terminated PDMS 4200 Da, and 0.5 mL of bis(2-ethylhexanoate)tin catalyst. The flask was again capped with a septum, purged with nitrogen for 10 minutes, and then put in a 60° C. shaker oven to react overnight. The packing was then rinsed with 250 mL toluene twice, 250 mL of methanol twice, and then dried in the vacuum oven at 40° C. for several hours before use.

### Example 15

### Functionalization of Graphite Packing for Improved Adhesion of Micellar Polymer Film

Graphite packing (25 mL) was etched first in boiling concentrated nitric acid for 4 hours. It was then rinsed thoroughly with water until pH was neutral and dried in vacuum oven at 40° C. overnight. The graphite was then added to a 100 mL round bottom flask with 30 mL dry toluene, 3 mL of HDI, and 0.1 mL of bis(2-ethylhexanaote)tin. The flask was capped with a septum, purged with nitrogen for 10 minutes, and then placed in a 60° C. shaker oven to react overnight. The graphite was rinsed with 50 mL toluene and 50 mL acetone (twice each) and then dried in vacuum oven for several hours. The graphite was then added to another 100 mL round bottom flask with 20 mL dry toluene, 1 mL of disilanol-terminated PDMS 550 Da, and 0.1 mL of bis(2-ethylhexanoate)tin. The flask was capped with a septum, purged with nitrogen for 10 minutes, and then placed in a 60° C. shaker oven to react overnight. The graphite was then rinsed with 50 mL toluene twice, 50 mL methanol twice, and dried in vacuum oven for several hours before use.

### Example 16

### Functionalization of Stainless Steel for Improved Adhesion of Micellar Polymer Film

The stainless steel (10 mL) was etched to remove the surface oxide layer by an acid pickling step by soaking the packing material in a 1.5% hydrofluoric acid, 7% nitric acid solution at 40° C. for 1 hour. The steel was then rinsed with water until pH was neutral and dried in the vacuum oven overnight.

### Example 16(a)

### Organic Phase Deposition

Freshly-etched steel was placed in a 100 mL round bottom flask with 25 mL dry toluene and 1 mL of N-(2-aminoethyl)-3-aminopropyltrimethoxysilane (AEAPTMS). The solution was stirred on a shaker plate at room temperature overnight. The steel was then rinsed with 20 mL toluene twice and 20 mL acetone twice and then dried in the vacuum oven for several hours before use.

### Example 16(b)

### Aqueous Phase Deposition

Freshly-etched steel was placed in a 100 mL round bottom flask with 4 mL of a 25 wt. % solution of aminoethylaminopropylsilsesquioxane-methylsilsesquioxane copolymer (AEAPSSQ-MSSQ) and 20 mL of water. This flask was stirred on a shaker plate at room temperature for 15 minutes. The steel was then rinsed with 20 mL water twice and then annealed in an oven at 100° C. for 10 minutes.

### Example 17

### Functionalization of Carbonic Anhydrase with Amine-Silanols

Carbonic anhydrase (CA) can be functionalized with silanol groups by coupling commercially available amine-silanols to the carboxylic acid groups of CA using carbodiimide chemistry. Below is an example using bCAII and N-(2-aminoethyl)-3-aminopropylsilanetriol (AEAPS).

AEAPS (5 mL, 25 wt. % solution in water) and 15 mL of water were added to a 100 mL beaker equipped with a stir bar. The pH of the solution was adjusted to ˜5.5 using concentrated hydrochloric acid. 400 mg of bCAII was then added with stirring. A catalytic amount of N-hydroxysulfosuccinimide sodium salt (sulfo-NHS; 10 mM=44 mg) was then added to the beaker. N-(3-dimethylaminopropyl)-N′-ethylcarbodiimide hydrochloride (101 mg, EDC; 40:1 molar ratio of EDC to CA) was then added to the beaker with stirring. The beaker was then wrapped in foil and allowed to react overnight at room temperature with stirring. The solution was then purified by dialysis in dialysis tubing with a molecular weight cut-off (MWCO) of 3400 Da against reverse osmosis (RO) water (5 L) for three days, changing the water twice a day. The AEAPS-functionalized bCAII was then lyophilized to dryness before use. The extent of functionalization can be manipulated by changing the amount of EDC used in the above procedure. Additionally, the type of amine-silanol and CA isoform can be changed in the above procedure. When using polymeric amine-silanols, dialysis tubing with a 12,000 Da MWCO was used. Table 17.1 contains the details for additional examples of successful amine-silanol functionalization of CA reactions.

TABLE 17.1
Synthetic details of additional functionalization reactions of
CA with amine-silanols

| Amine- | | | | | |
|---|---|---|---|---|---|
| silanol | | | | | Molar ratio |
| solution | | Water | Carbonic | EDC | of |
| (mL) | Type of amine silanol | (mL) | anhydrase | (mg) | EDC:CA |
| 5 | 25 wt. % solution of AEAPS | 15 | 400 mg; | 153 | 60:1 |
| | | | bCAII | | |
| 5 | 25 wt. % solution of AEAPS | 15 | 400 mg; | 52 | 20:1 |
| | | | bCAII | | |
| 5 | 25 wt. % solution of AEAPS | 15 | 400 mg; | 26 | 10:1 |
| | | | bCAII | | |
| 1 | 25 wt. % solution of AEAPS | 6 | 51 mg; | 14.5 | 45:1 |
| | | | hCAIV | | |
| 7.5 | 25 wt. % solution of | 7.5 | 300 mg; | 39 | 20:1 |
| | aminopropylsilsesquioxane (APSSQ) | | bCAII | | |
| 7.5 | 25 wt. % solution of | 7.5 | 300 mg; | 39 | 20:1 |
| | aminopropylsilsesquioxane- | | bCAII | | |
| | methylsilsesquioxane | | | | |
| | copolymer(APSSQ-MSSQ) | | | | |
| 7.5 | 25 wt. % solution of | 7.5 | 300 mg; | 39 | 20:1 |
| | aminoethylaminopropylsilsesquioxane- | | bCAII | | |
| | methylsilsesquioxane | | | | |
| | copolymer(AEAPSSQ-MSSQ) | | | | |
| 7.5 | 25 wt. % solution of AEAPSSQ-MSSQ | 7.5 | 300 mg; | 20 | 10:1 |
| | | | bCAII | | |
| 7.5 | 25 wt. % solution of AEAPSSQ-MSSQ | 7.5 | 300 mg; | 78 | 40:1 |
| | | | bCAII | | |

### Example 18

### Functionalization of Carbonic Anhydrase with Amine-Containing Polymers

Carbonic anhydrase (CA) can be functionalized with commercially available primary amine-containing polymers using carbodiimide chemistry. Below is an example using bCAII and branched poly(ethyleneimine) (PEI), which contains a mixture of primary, secondary, and tertiary amines at a 25:50:25 ratio.

13.3 g of PEI 1800 Da 50 wt. % solution in water was added to 100 mL beaker equipped with a Teflon stir bar and adjusted pH to ˜5.5 using concentrated hydrochloric acid. The final volume was brought to 20 mL using water. 200 mg of bCAII was then added, resulting in a 500:1 molar excess of PEI to CA. A catalytic amount of N-hydroxysulfosuccinimide sodium salt (sulfo-NHS; 10 mM=44 mg) was then added to the beaker. N-(3-dimethylaminopropyl)-N′-ethylcarbodiimide hydrochloride (51 mg, EDC; 40:1 molar ratio of EDC to CA) was then added to the beaker with stirring. The beaker was then wrapped in foil and allowed to react overnight at room temperature with stirring. The solution was then purified by dialysis in dialysis tubing with a MWCO of 12,000 Da against reverse osmosis (RO) water (5 L) for three days, changing the water twice a day. The PEI-functionalized bCAII was then lyophilized to dryness before use. The extent of functionalization could be manipulated by changing the amount of EDC used in the above procedure. Additionally, the type of amine-containing polymer could be changed in the above procedure. When using larger molecular weight amine-containing polymers, dialysis tubing with a 50,000 or 100,000 Da MWCO was used. Table 18.1 contains the details for additional examples of successful amine-containing polymer functionalization of CA reactions.

TABLE 18.1
Synthetic details of additional functionalization reactions of CA with amine-
containing polymers

| | Total | | | | |
|---|---|---|---|---|---|
| | volume | | Molar | | |
| | of | | ratio of | | |
| Amount and type of amine- | reaction | Carbonic | polymer | EDC | Molar ratio |
| containing polymer | (mL) | anhydrase | to CA | (mg) | of EDC:CA |
| 13.3 g of PEI 1800 Da 50 wt. % | 20 | 200 mg; | 500:1 | 26 | 20:1 |
| solution | | bCAII | | | |
| 13.3 g of PEI 1800 Da 50 wt. % | 20 | 200 mg; | 500:1 | 102 | 80:1 |
| solution | | bCAII | | | |
| 8.33 g of PEI 1800 Da 50 wt. % | 25 | 250 mg; | 250:1 | 96 | 60:1 |
| solution | | bCAII | | | |
| 3.33 g of PEI 1800 Da 50 wt. % | 25 | 250 mg; | 100:1 | 96 | 60:1 |
| solution | | bCAII | | | |
| 25 mL of a 50 wt. % PEI 10,000 Da | 25 | 200 mg; | 500:1 | 26 | 20:1 |
| solution in water | | bCAII | | | |
| 25 mL of a 50 wt. % PEI 10,000 Da | 25 | 200 mg; | 500:1 | 51 | 40:1 |
| solution in water | | bCAII | | | |
| 25 mL of a 50 wt. % PEI 10,000 Da | 25 | 200 mg; | 500:1 | 102 | 80:1 |
| solution in water | | bCAII | | | |
| 6.67 g of PEG 2000 Da diamine | 20 | 200 mg; | 500:1 | 26 | 20:1 |
| | | bCAII | | | |
| 6.67 g of PEG 2000 Da diamine | 20 | 200 mg; | 500:1 | 51 | 20:1 |
| | | bCAII | | | |
| 6.67 g of PEG 2000 Da diamine | 20 | 200 mg; | 500:1 | 102 | 20:1 |
| | | bCAII | | | |
| 0.24 g of amino-dextran 10,000 Da | 5 | 50 mg; | 100:1 | 9.6 | 30:1 |
| | | bCAII | of | | |
| | | | amine | | |
| | | | groups | | |
| | | | to CA | | |
| 0.24 g of amino-dextran 10,000 Da | 5 | 50 mg; | 100:1 | 19.2 | 60:1 |
| | | bCAII | of | | |
| | | | amine | | |
| | | | groups | | |
| | | | to CA | | |

### Example 19

### Scanning Electron Microscopy (SEM)

Micellar polymer mixture (3 mL) was vortexed with 0.1 mL of dibutyldilauryltin crosslinking catalyst in a glass vial until thoroughly mixed. This solution was then transferred to an acrylic mold (1 inch×1 inch×¼ inches deep) and annealed in an oven at 50° C. for 30 minutes. The edges of the molded polymer samples were removed by slicing with a razor blade, exposing the bulk material of the polymer, allowing more efficient sample staining Sections of the trimmed polymer samples were then placed in a solution of 1 wt. % phosphotungstic acid (PTA) and allowed to soak overnight. After soaking, the polymer was removed from solution and allowed to air dry. Thin sections were removed from the dried sample using a scalpel and mounted on an SEM substrate holder using double-sided carbon tape. Some samples were coated with a thin Au layer using a commercial sputter coater and Au target. Electron micrographs were collected using a field-emission SEM with both secondary electron and backscattering electron detectors. Acceleration voltages ranged from 2 keV-20 keV depending on the conductivity of the sample and the detector used.

### Example 20

### Sodium Dodecyl Sulfate Polyacrylamide Gel Electrophoresis (SDS PAGE)

SDS-PAGE was performed using 10 w/v % Bis(2-hydroxyethyl)-amino-tris(hydroxymethyl)-methane polyacrylamide gels (Invitrogen catalog #NP0302BOX). Samples were prepared in a lithium dodecylsulfate sample buffer and run on the gels with a 3-(N-morpholino)propanesulfonic acid (MOPS) running buffer. Separation occurred by constantly applying a 200 V field for 50 minutes. After separation, gels were fixed by shaking in a solution of RO water, methanol, and acetic acid (4:5:1) for 10 minutes. The fixing solution was then removed and the gels were shaken in a staining solution containing RO water, methanol, and commercially purchased staining solution (Stainer A) in ratios of 5.5:2:2 for an additional 10 minutes. After 10 minutes of initial staining, a second component, Stainer B, was added (5% by volume) to the original staining solution and the gel was shaken for a minimum of 3 hours. After staining the gels were rinsed in RO water for at least 7 hours.

### Example 21

### Isoelectric Focusing (IEF) Gel Electrophoresis

IEF was performed using 5 w/v % polyacrylamide gels (Invitrogen catalog #EC66552BOX). Samples were prepared in a premixed, 3-10 pH sample buffer and run on the gels using premixed cathode (glycerol and sodium azide) and anode (phosphoric acid) buffers. Running conditions included: (1) 100 V applied constantly for 1 hour; (2) 200 V applied constantly for 1 hour; (3) 500 V applied constantly for 30 minutes. After separation, the gels were fixed by shaking in a solution of trichloroacetic acid (12 wt. %), sulfosalicyclic acid (3.5 wt. %), and water for 1 hour. After fixation, the gels were stained by shaking in a solution containing RO water, methanol, and a commercially purchased colloidal staining solution.

### Example 22

### Fourier Transform Infrared (FTIR) Spectroscopy

Attenuated total reflectance (ATR) FTIR was performed on surface-functionalized steel, graphite, glass, and ceramic samples. Each spectrum consisted of 100 total scans through a range of 750-4000 cm⁻¹ with a resolution setting of 2 cm⁻¹.

### Example 23

### Immobilization of bCAII in PHMS-g-PEG Micellar Polymer Pellets

Sample 23(a).

A micellar polymer mixture was made with the following components: 3 mL of PHMS-g-PEG (62 wt. % PEG), 3 mL of PHMS-g-PEG (71 wt. % PEG), 3 mL of PHMS-g-PEG (82 wt. % PEG), 9 mL of disilanol-terminated PDMS 4200 g/mol, 2 mL of PHMS 2250 g/mol, and 2 mL of a 50 wt. % solution of silanol trimethylsilyl-modified Q resin in decamethylcyclopentasiloxane. This micellar polymer mixture had 29 wt. % PEG overall.

100 mg of bCAII powder was vortexed with 2.4 mL of the above micellar polymer mixture in a glass vial until thoroughly mixed. 75 μL of dibutyldilauryltin crosslinking catalyst was then added and vortexed to mix. This solution was transferred into an acrylic mold of cylinders (¼ inches deep×⅛ inches in diameter) using a transfer pipet and annealed in an oven at 60° C. for 1 hour. The polymer pellets were then removed from the mold and then equilibrated in 1M potassium carbonate overnight before testing.

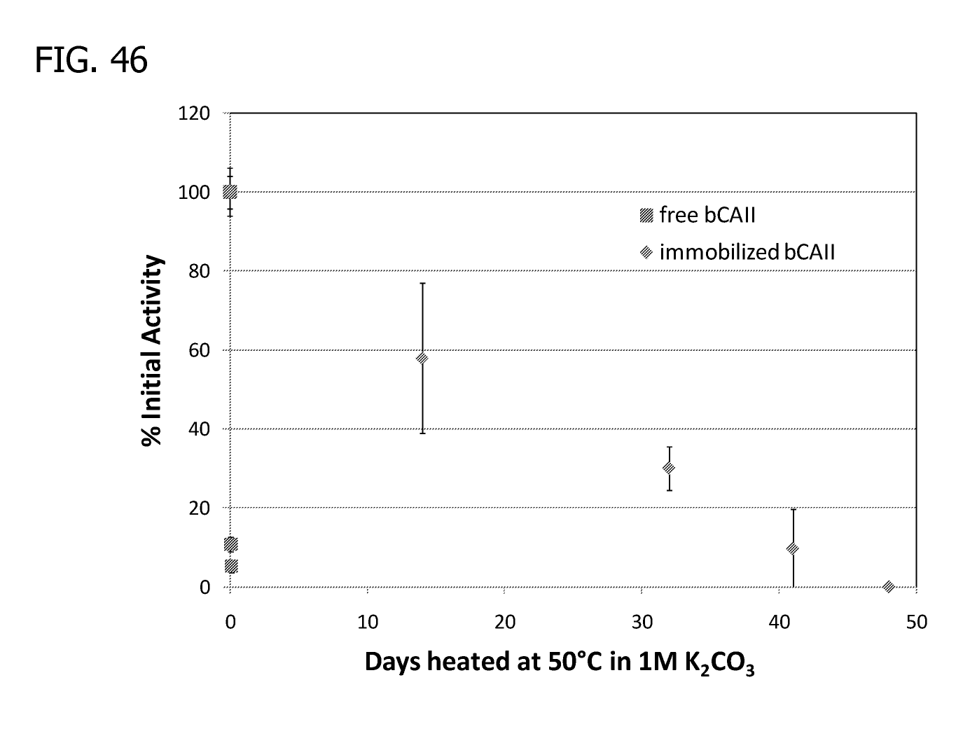

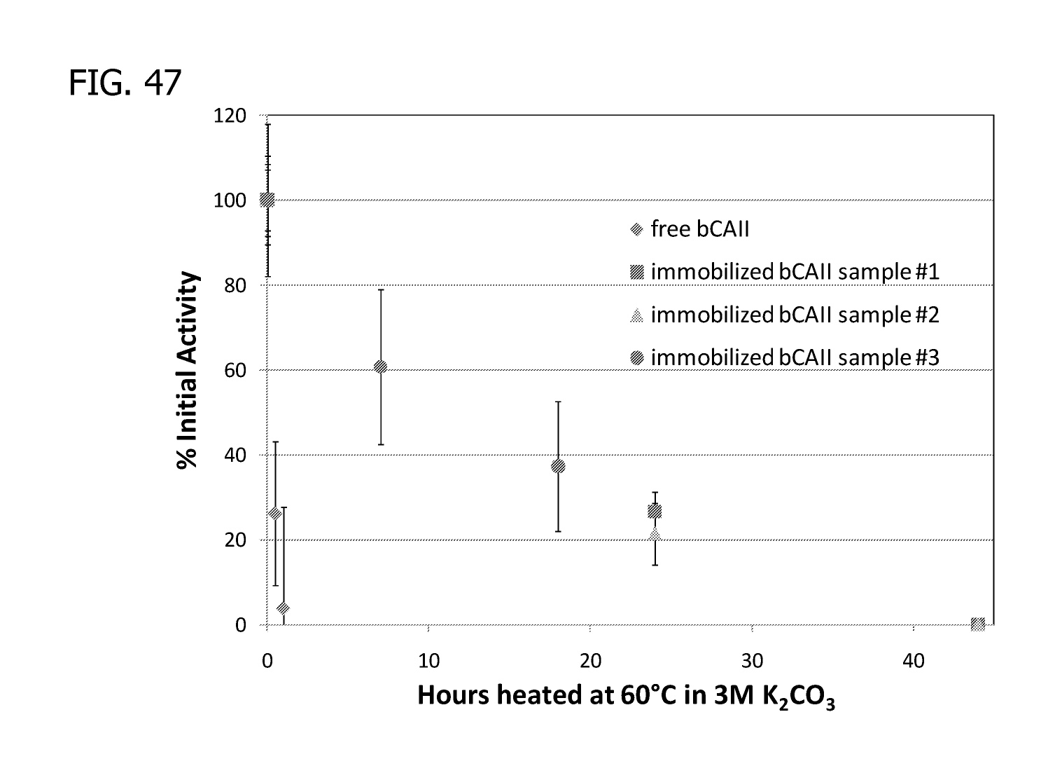

The pellets were then divided into 4 aliquots of equal volume for stability testing at elevated temperatures, ionic strengths, and pH, shown in the figures below. FIG. 46 shows a lifetime comparison of bCAII free in solution in 1M potassium carbonate at 50° C. vs. immobilized in the pellets as described above and showed ˜650 times improvement in lifetime upon immobilization. FIG. 47 shows a lifetime comparison of bCAII free in solution in 3M potassium carbonate at 60° C. vs. immobilized in the pellets as described above and showed ˜50 times improvement in lifetime upon immobilization.

The above sample had an enzyme loading of 4 wt. % in the micellar polymer pellets. The enzyme loading can be adjusted by changing the amount of enzyme powder added before crosslinking, with an upper limit of ˜10%. Above this value, the enzyme/polymer mixture becomes too viscous to be able to transfer it easily into an acrylic mold with such small dimensions.

Sample 23(b).

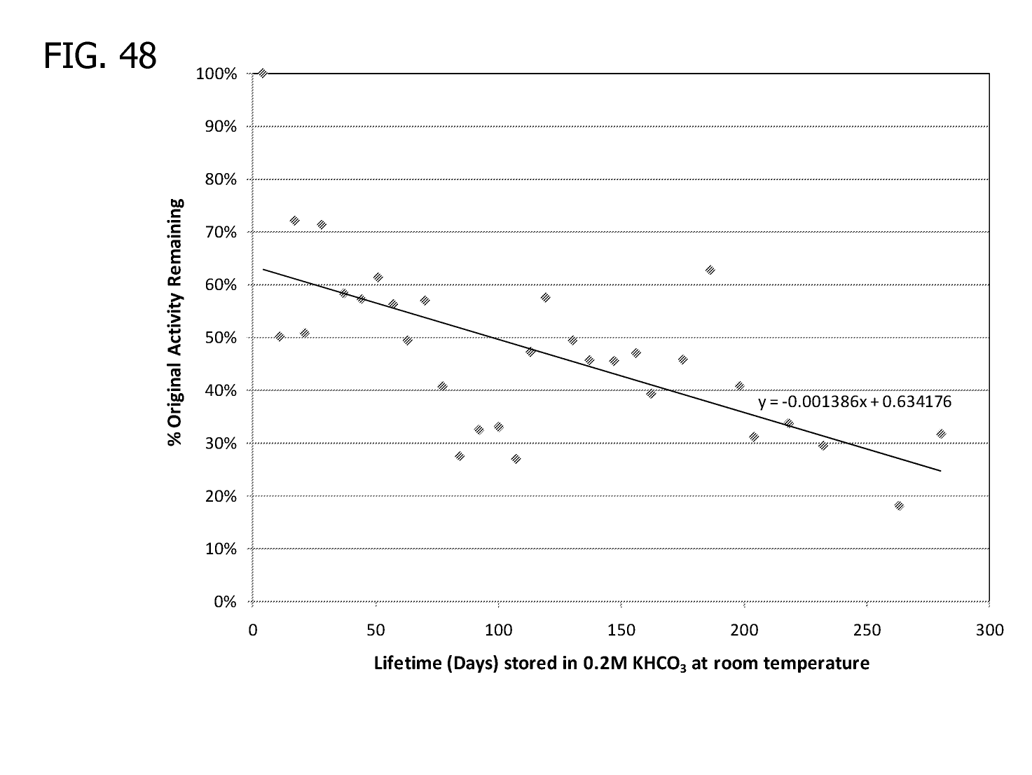

In a glass vial, 1 mL of PHMS-g-PEG (62 wt. % PEG), 1 mL of disilanol-terminated PDMS 4200 g/mol, and 0.1 mL of 50 wt. % solution of silanol trimethylsilyl-modified Q resin in decamethylcyclopentasiloxane were added and vortexed together (29 wt. % PEG overall). bCAII powder (50 mg) was added to this micellar polymer mixture and vortexed to mix. Dibutyldilauryltin catalyst (80 μL) was vortexed into this mixture, and it was transferred via pipet to an acrylic mold of cylinders (¼ inches deep× 3/16 inches in diameter). The mold was placed in the refrigerator at 4° C. to allow the polymer to crosslink overnight. The pellets were then removed from the mold and equilibrated in 0.2M potassium bicarbonate overnight before analysis. FIG. 48 shows the lifetime of bCAII immobilized in the above pellets as assayed by the pH stat and stored in 0.2M potassium bicarbonate at room temperature in between analyses. This data predicts an enzyme lifetime of 460 days under these conditions, according to a linear regression analysis of the activity decay of the sample.

Sample 23(c).

In a 50 mL glass beaker, 3 mL of PHMS-g-PEG (62 wt. % PEG), 3 mL of PHMS-g-PEG (71 wt. % PEG), 3 mL of PHMS-g-PEG (82 wt. % PEG), 9 mL of disilanol-terminated PDMS 4200 g/mol, 2 mL of PHMS 2250 g/mol, and 2 mL of a 50 wt. % solution of silanol trimethylsilyl-modified Q resin in decamethylcyclopentasiloxane were added and vortexed thoroughly. This micellar polymer mixture had 29 wt. % PEG overall. To this micellar polymer mixture, 300 mg of bCAII powder was added and vortexed to mix. Dibutyldilauryltin crosslinking catalyst (0.6 mL) was then vortexed in, and this enzyme/polymer mixture was poured into the bottom of a multi-well acrylic mold of cylinders (¼ inches deep×⅛ inches in diameter). The upper piece of the mold was placed on top, forcing the polymer liquid into the cylinder wells. The polymer was then crosslinked at room temperature for several hours before removing from the acrylic mold and transferring to a solution of 2M KHCO₃/1M K₂CO to equilibrate overnight. This procedure was repeated two more times to get ˜50 mL of polymer pellets for analysis in a batch reactor, described in more detail below.

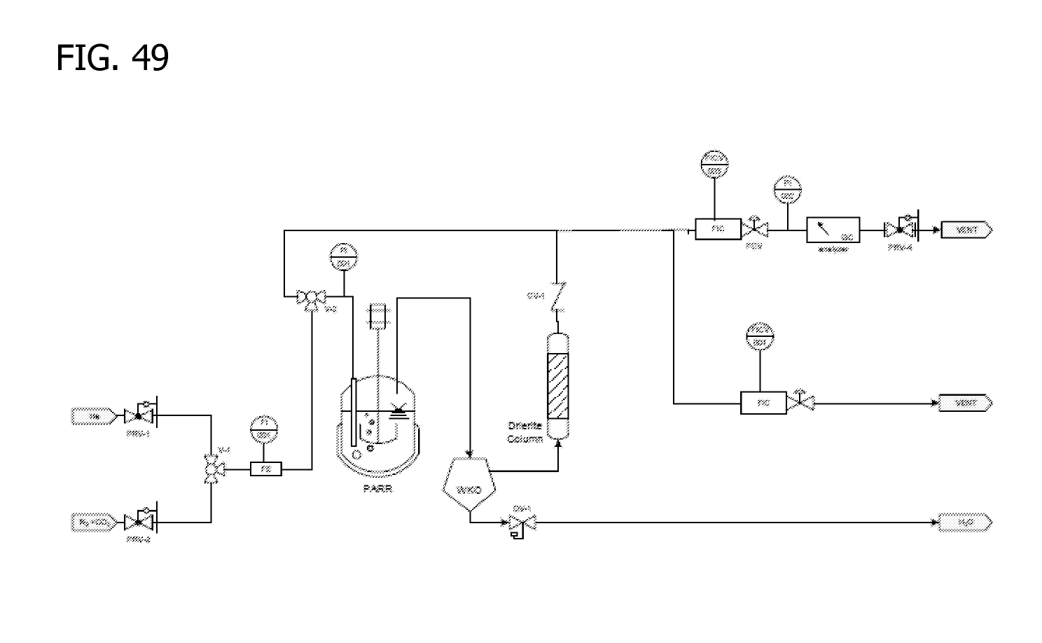

To test multiple enzyme/polymer systems for immobilized activity, small batches (50 mL) were tested in a batch reactor. The batch reactor allowed the immobilized enzyme sample to be placed in a vessel submerged in a HCO₃⁻/CO₃²⁻ solution and purged with gas. A schematic of the batch reactor analysis system is shown in FIG. 49. Ceramic packing material (50 mL) coated with a polymer containing the immobilized enzyme sample was placed in the batch reactor vessel. A 1M HCO₃⁻/0.5M CO₃²⁻ solution, pH ˜9.4, (30 mL) was then added to the vessel containing the packing material. An 85% N₂/15% CO₂ gas mixture was purged into the vessel at a flow rate of 100 SCCM at 30 psig. The gas mixture exiting the batch reactor passed through a water knock out (WKO) and a Drierite column before being fed, by a mass flow controller, to a gas chromatograph (GC) equipped with a thermal conductivity detector (TCD). The CO₂ in the mixture was separated from other gas components by the GC and quantitated using the TCD.

Enzyme activity was calculated based on the magnitude of CO₂ conversion and was reported as the average activity (rate) in μmol/L·min. This average was calculated by summing the quantity (μmol) of CO₂ converted over a 15 minute time frame and dividing this sum by the time interval (15 min.) to yield activity units of μmol/L·min. The volume unit (L) in the activity calculation represents the rate achieved per liter of packing material (50 mL of packing material was used for these experiments). All activities reported in the figures have been background subtracted (i.e., the rate associated with CO₂ hydration in the solvent alone has been subtracted from the total rate achieved by the enzyme sample).

At the end of the run a pH measurement was made on the reactor solution. A single pH measurement was taken at the end of the run because the reactor system was not outfitted with the appropriate equipment to continually monitor pH during the analysis. All analyses were conducted without stirring and at room temperature.

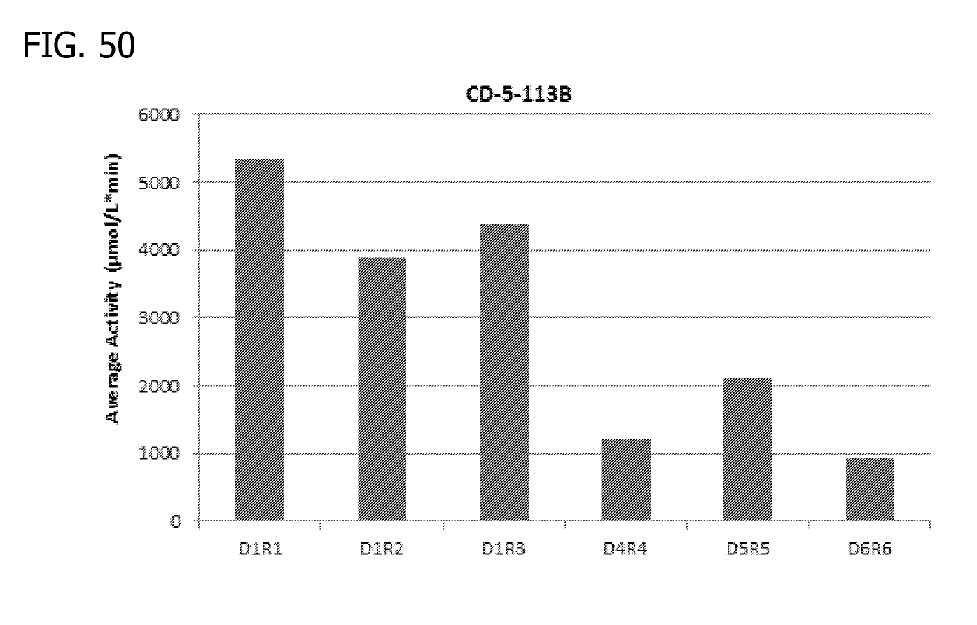

FIG. 50 shows the batch reactor analysis for bCAII enzyme immobilized within PHMS-g-PEG micellar polymer pellets (sample preparation described above). The notation in this figure (DXRY) represents the day of analysis (X) and cumulative run total (Y).

### Example 24

### Immobilization of bCAII in PHMS-g-PEG Micellar Polymer Coatings on Ceramic

A micellar polymer mixture was made with the following components: 3 mL of PHMS-g-PEG (62 wt. % PEG), 3 mL of PHMS-g-PEG (71 wt. % PEG), 3 mL of PHMS-g-PEG (82 wt. % PEG), 9 mL of disilanol-terminated PDMS 4200 g/mol, 2 mL of PHMS 2250 g/mol, and 2 mL of a 50 wt. % solution of silanol trimethylsilyl-modified Q resin in decamethylcyclopentasiloxane. This micellar polymer mixture had 29 wt. % PEG overall.

The above micellar polymer mixture (1.5 mL) was placed in a 150 mL glass beaker with 0.3 mL of an aqueous bCAII solution (˜300 mg/mL) and stirred thoroughly to mix. Bis(2-ethylhexanoate)tin crosslinking catalyst (60 μL) was stirred in quickly to mix, followed by 30.3 g of PDMS-functionalized Berl saddle ceramic packing pieces (˜180 pieces=30 mL; specific surface area of 6.28 cm²/cm³). The ceramic pieces were thoroughly stirred to coat them with the enzyme/polymer mixture and then spread out on a piece of stainless steel mesh to prevent pieces adhering together during crosslinking. The mesh was then transferred into a 50° C. oven to allow the polymer coating to crosslink for 30 minutes. This procedure was repeated to get the 50 mL of polymer-coated packing required for batch reactor testing. The sample was equilibrated in 2M KHCO₃/1M K₂CO₃ overnight prior to analysis.

The above sample had a 5.7 wt. % enzyme loading in the micellar polymer mixture. This enzyme loading could be changed in several ways. The amount of aqueous enzyme solution added to the micellar polymer mixture could be adjusted, with an upper limit of ˜30% (0.3 mL of aqueous enzyme solution to 1 mL micellar polymer mixture). Adding more water than that to the micellar polymer mixture hindered crosslinking and prevented good coating formation. Also, the concentration of the enzyme solution could be changed. Instead of using an aqueous enzyme solution, the enzyme could also be added to the micellar polymer mixture as a powder before crosslinking into a coating.

Based on the measured transfer efficiency of ˜80% in the above method (i.e., 80% of the enzyme/polymer mixture was actually coated onto the ceramic pieces) and the specific surface area of the ceramic, the film thickness of this coating was estimated to be ˜60 μm. This thickness could be easily adjusted by changing the volume/number of ceramic pieces added to the same amount of enzyme/micellar polymer mixture.

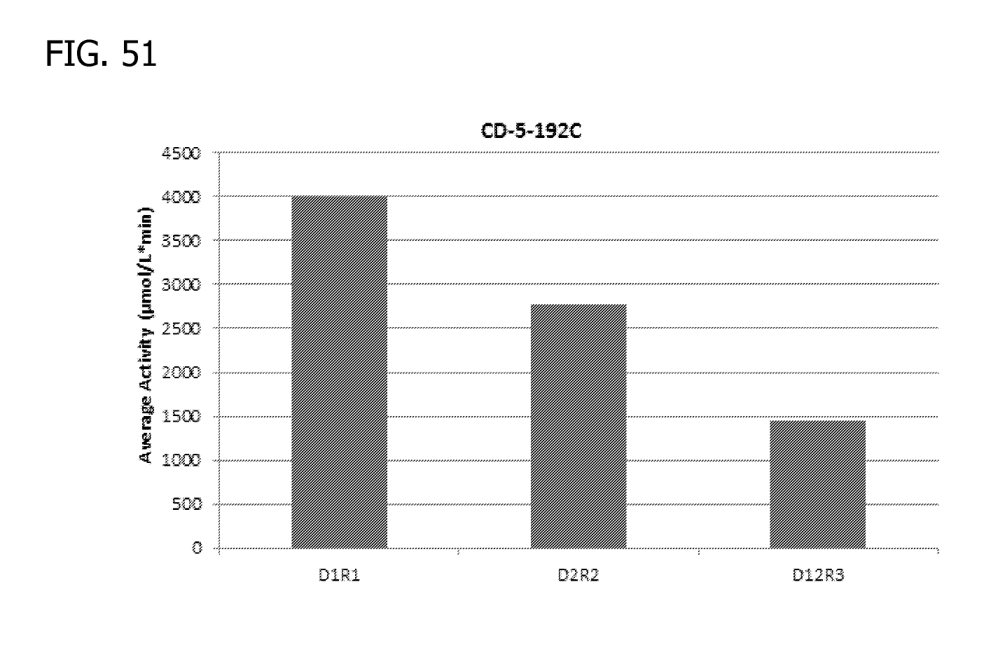

FIG. 51 shows the batch reactor analysis over the course of 12 days for the sample described above, 5.7 wt. % bCAII immobilized in PHMS-g-PEG ˜60 μm thick micellar polymer coating on ceramic.

### Example 25

### Immobilization of AEAPS-Functionalized bCAII in PHMS-g-PEG Micellar Polymer Coatings on Ceramic

A micellar polymer mixture was made with the following components: 4.5 mL of PHMS-g-PEG (62 wt. % PEG), 4.5 mL of PHMS-g-PEG (82 wt. % PEG), 9 mL of disilanol-terminated PDMS 4200 g/mol, 2 mL of PHMS 2250 g/mol, and 2 mL of a 50 wt. % solution of silanol trimethylsilyl-modified Q resin in decamethylcyclopentasiloxane. This micellar polymer mixture had 29 wt. % PEG overall.

The above micellar polymer mixture (1.5 mL) was placed in a 150 mL glass beaker with 0.3 mL of a aqueous AEAPS-functionalized bCAII solution (˜250 mg/mL using AEAPS-functionalized bCAII with an EDC:CA molar ratio of 10:1) and stirred thoroughly to mix. Bis(2-ethylhexanoate)tin crosslinking catalyst (60 μL) was stirred in quickly to mix, followed by 30.3 g of PDMS-functionalized Berl saddle ceramic packing pieces (˜180 pieces=30 mL; specific surface area of 6.28 cm²/cm³). The ceramic pieces were thoroughly stirred to coat them with the enzyme/polymer mixture and then spread out on a piece of stainless steel mesh to prevent pieces adhering together during crosslinking. The mesh was then transferred into a 50° C. oven to allow the polymer coating to crosslink for 30 minutes. This procedure was repeated to get the 50 mL of polymer-coated packing required for batch reactor testing. The sample was equilibrated in 2M KHCO₃/1M K₂CO₃ overnight prior to analysis.

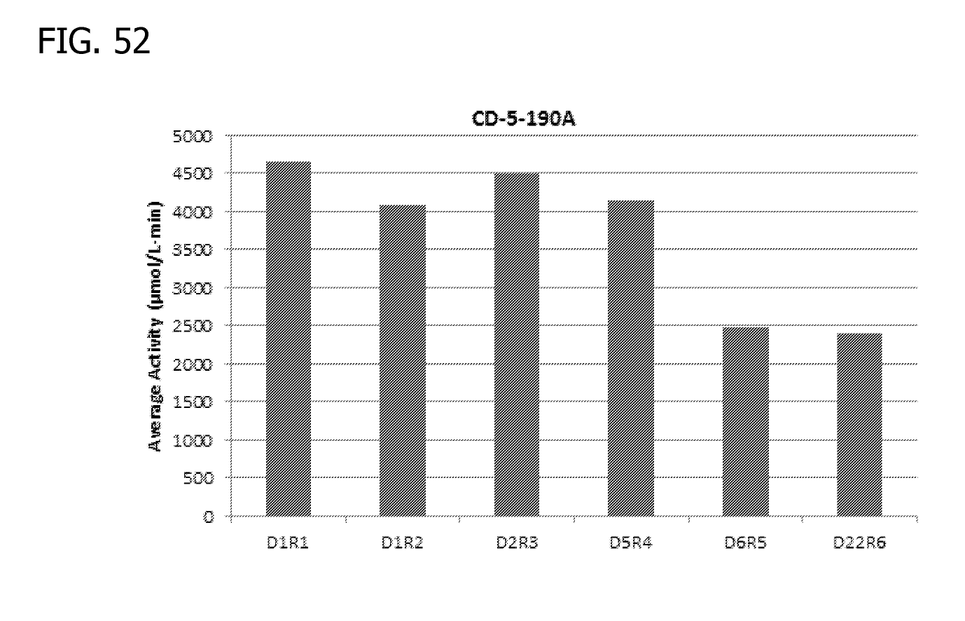

FIG. 52 shows the batch reactor analysis over the course of 22 days for the sample described above, 4.8 wt. % AEAPS-functionalized bCAII immobilized in PHMS-g-PEG ˜60 μm thick micellar polymer coating on ceramic.

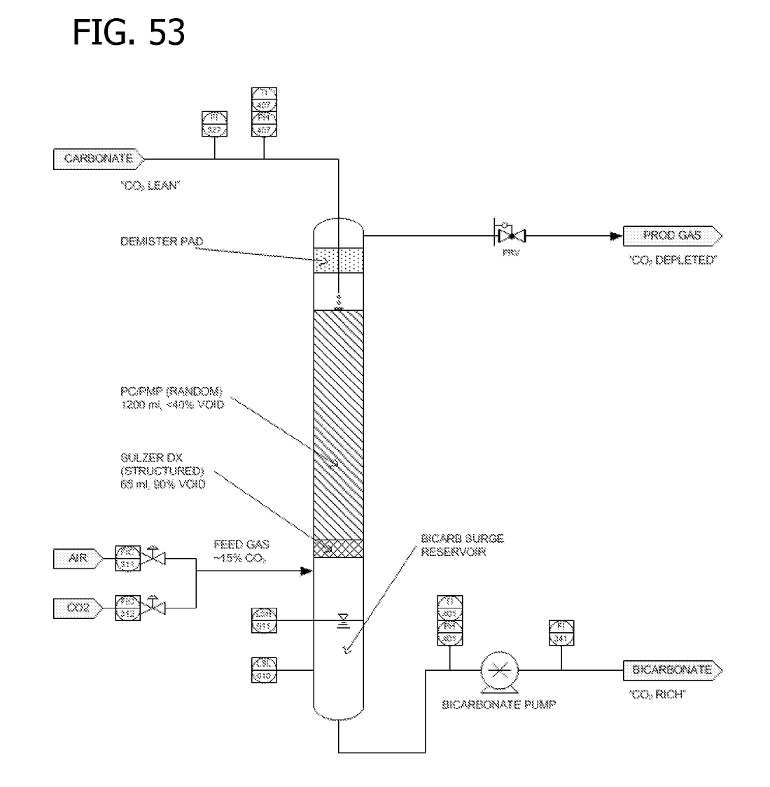

The above procedure was also scaled up for analysis in a closed-loop reactor. A total of 47 batches of enzyme-containing micellar polymer were coated on ceramic pieces to get a total of 1.2 L of coated ceramic packing to load in the absorber unit of a closed-loop reactor. A schematic of the absorber side of the closed-loop reactor is provided in FIG. 53.

After operating the closed-loop reactor for an initial 20 hour run, the reactor was halted and the packing removed to check for activity in the batch reactor. Afterwards, the sample was reloaded into the closed loop reactor and operated for 100 hours with the following set point conditions: inlet absorber pH of 9.3 (correlating to a reboiler temperature of 107° C.), a liquid flow of 64 mL/min through the absorber/reboiler, a gas flow of 6 L/min of 15 vol % CO₂ (balance air), absorber inlet temperature of 35° C., and an absorber pressure of 5 psig.

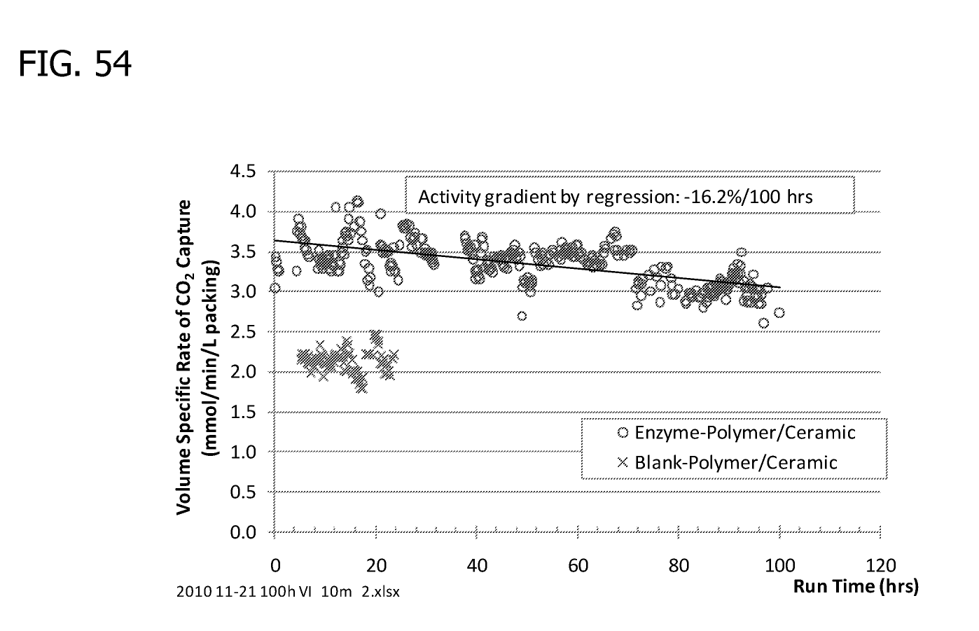

The data for that 100-hour continuous run is presented in FIG. 54 as compared to a 24-hour run with polymer-coated ceramic packing that did not contain enzyme (blank-polymer/ceramic).

### Example 26

### Immobilization of AEAPS-Functionalized bCAII in PHMS-g-PEG Micellar Polymer Coatings on Ceramic

A micellar polymer mixture was made with the following components: 3 mL of PHMS-g-PEG (62 wt. % PEG), 3 mL of PHMS-g-PEG (71 wt. % PEG), 3 mL of PHMS-g-PEG (82 wt. % PEG), 9 mL of disilanol-terminated PDMS 4200 g/mol, 0.6 mL of PHMS 2250 g/mol, and 1.4 mL of a 50 wt. % solution of silanol trimethylsilyl-modified Q resin in decamethylcyclopentasiloxane. This micellar polymer mixture has 32 wt. % PEG overall.

The above micellar polymer mixture (1.5 mL) was placed in a 150 mL glass beaker with 0.3 mL of a aqueous AEAPS-functionalized bCAII solution (˜250 mg/mL using AEAPS-functionalized bCAII with an EDC:CA molar ratio of 10:1) and stirred thoroughly to mix. Bis(2-ethylhexanoate)tin cross-linking catalyst (60 μL) was stirred in quickly to mix, followed by 30.3 g of PDMS-functionalized Berl saddle ceramic packing pieces (˜180 pieces=30 mL; specific surface area of 6.28 cm²/cm³). The ceramic pieces were thoroughly stirred to coat them with the enzyme/polymer mixture and then spread out on a piece of stainless steel mesh to prevent pieces adhering together during cross-linking. The mesh was then transferred into a 50° C. oven to allow the polymer coating to cross-link for 30 minutes. This procedure was repeated to get the 50 mL of polymer-coated packing required for batch reactor testing. The sample was equilibrated in 2M KHCO₃/1M K₂CO₃ overnight prior to analysis.

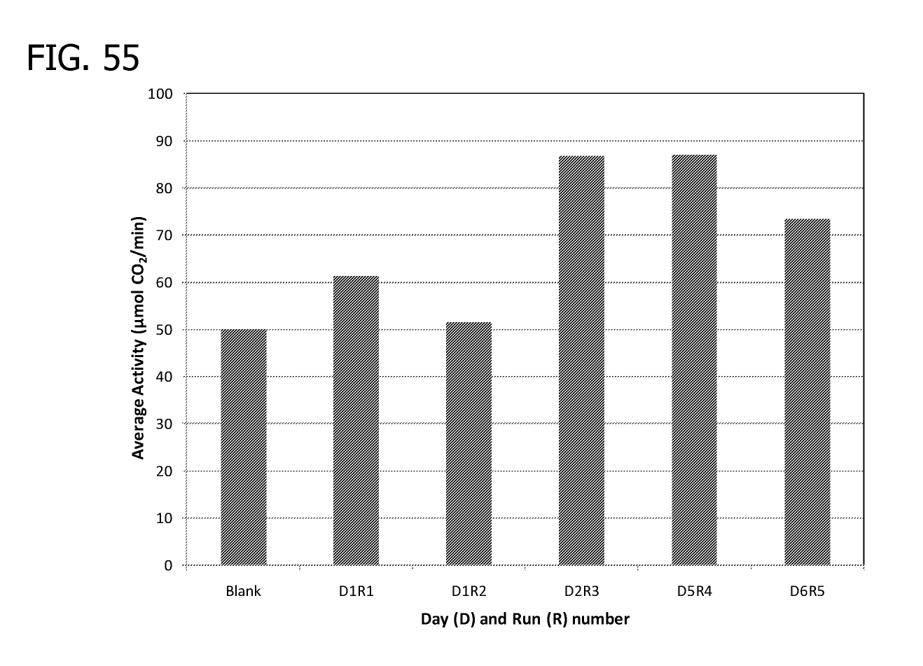

FIG. 55 shows the batch reactor analysis over the course of 6 days for the sample described above, 4.8 wt. % AEAPS-functionalized bCAII immobilized in PHMS-g-PEG ˜60 μm thick micellar polymer coating on ceramic.

### Example 27

### Immobilization of PEI-Functionalized bCAII in PHMS-g-PEG Micellar Polymer Coatings on Ceramic

A micellar polymer mixture was made with the following components: 3 mL of PHMS-g-PEG (62 wt. % PEG), 3 mL of PHMS-g-PEG (71 wt. % PEG), 3 mL of PHMS-g-PEG (82 wt. % PEG), 9 mL of disilanol-terminated PDMS 4200 g/mol, 2 mL of PHMS 2250 g/mol, and 2 mL of a 50 wt. % solution of silanol trimethylsilyl-modified Q resin in decamethylcyclopentasiloxane. This micellar polymer mixture had 29 wt. % PEG overall.

The above micellar polymer mixture (1.5 mL) was placed in a 150 mL glass beaker with 0.3 mL of a aqueous PEI-functionalized bCAII solution (˜300 mg/mL using PEI 1800 Da-functionalized bCAII with an PEI:EDC:CA molar ratio of 100:60:1) and stirred thoroughly to mix. Bis(2-ethylhexanoate)tin crosslinking catalyst (60 μL) was stirred in quickly to mix, followed by 30.3 g of PDMS-functionalized Berl saddle ceramic packing pieces (˜180 pieces=30 mL; specific surface area of 6.28 cm²/cm³). The ceramic pieces were thoroughly stirred to coat them with the enzyme/polymer mixture and then spread out on a piece of stainless steel mesh to prevent pieces adhering together during crosslinking. The mesh was then transferred into a 50° C. oven to allow the polymer coating to crosslink for 30 minutes. This procedure was repeated to get the 50 mL of polymer-coated packing required for batch reactor testing. The sample was equilibrated in 2M KHCO₃/1M K₂CO₃ overnight prior to analysis.

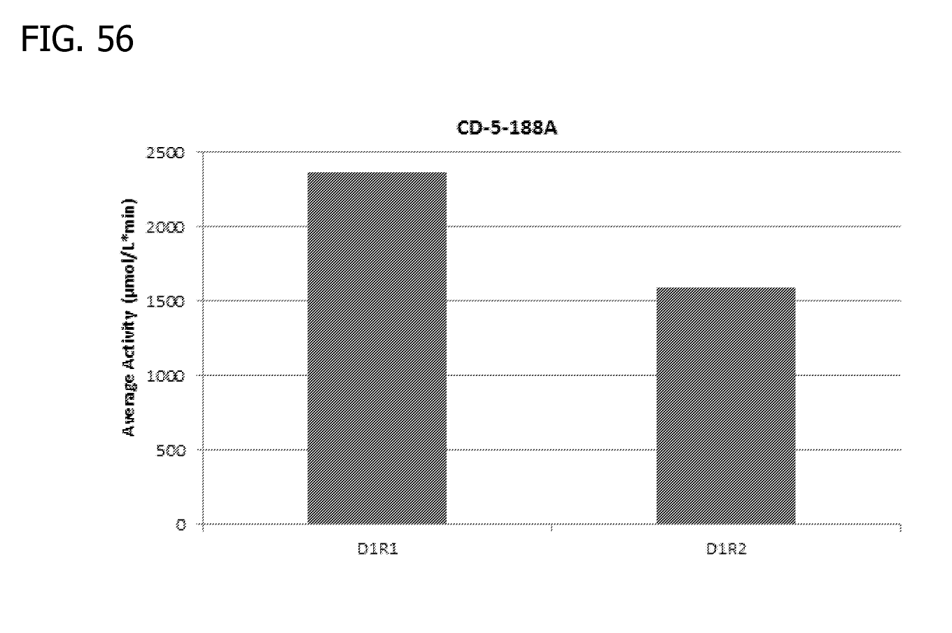

FIG. 56 shows the batch reactor analysis of PEI-functionalized bCAII immobilized within PHMS-g-PEG ˜60 μm thick micellar polymer coating at 5.7 wt. % enzyme loading on ceramic. Activity values have been background subtracted.

### Example 28

### Synthesis of Allyl-PEG-Succinimide

The synthesis of allyl-PEG-succinimide was achieved in four steps starting with commercially available allyl-PEG-OH (8-12 ethylene oxide units; average molecular weight=500 g/mol). Each of the four steps is described in detail below.

### Example 28(a)

### Synthesis of Allyl-PEG-500 Nitrile

A 500 mL 3 neck round bottom flask equipped with a mechanical stirrer and a thermocouple was charged with allyloxy(polyethylene oxide) (50 g, 0.10 mol) and 200 mL of water. The resulting solution was chilled to 0° C. Sodium hydroxide (6.0 g, 0.15 mol) was added and the solution was stirred at 0° C. for 10 minutes. Acrylonitrile (11.4 mL, 0.17 mol) was then added dropwise over 30 minutes. The reaction mixture was allowed to warm to room temperature and then stirred for 20 hours. After this time, the reaction mixture was neutralized with 3M HCl (25 mL). Chloroform was added and the layers were separated. The aqueous layer was extracted with chloroform (2×300 mL). The combined organic layer was washed once with brine, dried over anhydrous magnesium sulfate, filtered and evaporated under reduced pressure to give 53 g (96% yield) of allyl PEG-500 nitrile as a clear liquid. ¹H NMR (CDCl₃) δ 5.89 (m, 1H), 5.20 (dd, J=1.5, 18.7 Hz, 2H), 3.99 (m, 2H), 3.68 (m, 2H), 3.64 (m, 32-34H), 2.61 (m, 2H).

### Example 28(b)

### Synthesis of Allyl-PEG-500 Amide

A 500 mL 3 neck round bottom flask equipped with a mechanical stirrer and a thermocouple was charged with allyl PEG nitrile (50 g, 0.094 mol) and 200 mL of water. The resulting solution was chilled to 0° C. Potassium hydroxide (5.1 g, 0.094 mol) was added and the solution was stirred at 0° C. flask for 10 minutes. A 30% hydrogen peroxide solution (68 mL) was then added dropwise. The reaction mixture appeared clear after all the peroxide had been added. The reaction mixture was allowed to stir from 0° C. to room temperature for 20 hours. After this time, the reaction mixture was cooled to 0° C. and treated with 3M hydrochloric acid (HCl) (˜20 mL) to ˜pH 4. Dichloromethane was added and the layers were separated. The aqueous layer was extracted with dichloromethane (3×200 mL). The combined organic layer was washed once with brine, dried over anhydrous magnesium sulfate, filtered and evaporated under reduced pressure to give 48 g (92% yield) of allyl-PEG-500 amide. ¹H NMR (CDCl₃) δ 5.89 (m, 1H), 5.23 (dd, J=1.5, 51.4 Hz, 2H), 4.01 (m, 2H), 3.72 (m, 2H), 3.62 (m, 32-34H), 2.63 (m, 2H).

### Example 28(c)

### Synthesis of Allyl-PEG-Propionic Acid

A 500 mL 3 neck round bottom flask equipped with a mechanical stirrer and a thermocouple was charged with allyl-PEG-500 amide (11 g, xx mol) and 200 mL of water. The resulting solution was chilled to 0° C. Powdered sodium hydroxide (22 g, 0.55 mol) was added and the solution was stirred for 20 h from 0° C. to room temperature. After this time, the reaction mixture was cooled to 0° C. and treated with 3 N HCl (˜90 mL) to pH 3. The reaction mixture was then transferred to a reparatory funnel and extracted with dichloromethane (3×450 mL). The combined organic layer was washed with brine (1×200 mL). The organic phase was dried over anhydrous magnesium sulfate, filtered, and evaporated under reduced pressure to give 9.5 g (84% yield) of allyl-PEG-propionic acid as a clear oil. ¹H NMR (CDCl₃) δ 5.91 (m, 1H), 5.56 (m, 2H), 4.03 (m, 2H), 3.76 (m, 2H), 3.64 (m, 32-34H), 2.61 (m, 2H).

### Example 28(d)

### Synthesis of Allyl-PEG-Succinimide

A 250 mL 3 neck round bottom flask equipped with a mechanical stirrer and a thermocouple was charged with allyl PEG propionic acid (9.5 g) and 50 mL of dichloromethane. The resulting solution was chilled to 0° C. N-Hydroxy-succinimide (2.3 g, 0.020 mol) was added and the solution was stirred for 10 minutes. 1-Ethyl-3-(3-dimethylaminopropyl)carbodiimide hydrochloride (EDC) (4.79 g, 0.025 mol) was added and the reaction mixture was stirred at 0° C. for 2 hours and then allowed to warm to room temperature and stirred for 20 hours. After this time, the reaction mixture was diluted with 150 ml of dichloromethane and then washed with water (3×100 mL). The organic layer was washed with brine, dried over anhydrous magnesium sulfate, filtered, and evaporated under reduced pressure to give 9.0 g (82% yield) of allyl-PEG-succinimide as a clear oil. ¹H NMR (CDCl₃) δ 5.90 (m, 1H), 5.26 dd (J=1.57, 17.2 Hz, 1H), 5.17 (dd, J=1.17, 10.4 Hz, 1H), 4.01 (m, 2H), 3.63, (m, 36-40H), 2.89 (t, J=6.44 Hz, 2H), 2.83 (bs, 2H), 2.80 (s, 2H), 2.63 (m, 2H).

### Example 29

### Grafting Reaction of Allyl-PEG-Monomethyl Ether and Allyl-PEG-Succinimide onto PHMS Using Platinum Oxide

Functional groups that can couple to carbonic anhydrase could be incorporated into the micellar polymer by grafting allyl-PEGs that had been previously functionalized with these reactive moieties. For example, the synthesis below describes in detail the addition of a succinimide group into the micellar polymer.

A dry 250 mL 3 neck round bottom flask was equipped with a teflon stir bar, a thermostat and heating mantle, a condenser with bubbler, and a nitrogen line. To this flask were added 4 g of previously synthesized PHMS-g-PEG, 60 wt. % PEG in 8 mL toluene which had been dried over molecular sieves overnight. The reaction mixture was purged with nitrogen for 15 minutes, then heated to 65° C. and held at this temperature. Platinum oxide (40 mg) was added and then the suspension was stirred for 10 minutes. A solution of allyl-PEG-succinimide (6.0 g) in 10 mL of dry toluene was added dropwise over 10 minutes. The reaction mixture was then heated to 80° C. and held at this temperature for about 18 hours until the reaction was complete. The reaction mixture was then cooled to room temperature, diluted with 30 mL of toluene, and filtered via gravity filtration to remove the platinum catalyst. The toluene was removed under reduced pressure to yield poly(hydrogenmethylsiloxane)-graft-[poly(ethylene glycol)+poly(ethylene glycol)-succinimide] (PHMS-g-PEG/PEG-succinimide) as a viscous, clear liquid. The average molecular weight, PEG content, and succinimide content was determined via ¹H NMR spectroscopy in deuterated chloroform. By comparing the methyl peak (3.4 ppm), succinimide peak (2.8 ppm), and silicon hydride peak (4.7 ppm) and knowing that each PHMS chain had 35 silicon hydride groups initially, it was determined that this graft copolymer had an average molecular weight of 10,400 g/mol, 76 wt. % PEG, and 7 succinimide moieties per PHMS chain. The PEG content and number of succinimide moieties could be manipulated by changing the ratios of starting materials.

### Example 30

### Functionalization of Ceramic Packing for Improved Adhesion of Micellar Polymer Films

Ceramic packing (500 mL) was etched in 10% aqueous HF solution overnight (for silicate ceramics) with stirring, then rinsed thoroughly with water, transferred into a beaker containing a 1:1:3 mixture of concentrated ammonium hydroxide: 30 wt. % hydrogen peroxide solution:water and then heated at 80° C. for 2 hours. The packing was rinsed thoroughly with water until the pH of the rinse was neutral, and then dried in an oven at 80° C. overnight. The ceramic was then added to a 1 L round bottom flask containing 400 mL of dry hexane and 5.8 mL allyltrichlorosilane (˜100 mM). The flask was capped with a septum, purged with nitrogen for 10 minutes, and then put in a room temperature shaker to react overnight. The packing was then rinsed three times with 250 mL of hexane, three times with 250 mL of acetone, three times with 250 mL of reverse osmosis (RO) water, and dried in an oven at 80° C. for several hours. The ceramic was characterized via FTIR before use.

### Example 31

### Functionalization of Carbonic Anhydrase with Allyl-PEG-Succinimide or N-Acryloxysuccinimide

Carbonic anhydrase (CA) could be functionalized with allyl groups by coupling allyl-succinimides to the amine groups of CA. The example below illustrates the coupling of Novozyme CA and allyl-PEG-succinimide.

A 150 mL beaker was filled with 40 mL of 41.4 mg/mL dialyzed Novozyme CA (x mmol) and 40 mL of 200 mM phosphate buffer, pH 7.5. A Teflon stir bar was added and the solution was stirred to homogenize. Allyl-PEG succinimide (0.99 mL, x mmol) (30:1 molar ratio of allyl-PEG-succinimide to CA) was then added dropwise to the beaker with stirring. The reaction mixture was stirred for 2 hours at room temperature. The solution was purified by dialysis in dialysis tubing with a MWCO of 6000-8000 Da against 5 mM phosphate buffer, pH 7.5 for two days, changing the buffer twice. The allyl-PEG functionalized CA was then lyophilized to dryness before use. The extent of functionalization could be manipulated by changing the amount of allyl-PEG succinimide used in the above procedure.

A similar procedure was used for the functionalization of Novozyme CA with N-acryloxysuccinimide. In this analogous procedure, N-acryloxysuccinimide was dissolved in minimal amounts of dimethyl sulfoxide and then added dropwise to the enzyme in phosphate buffer. Alternatively, N-acryloxysuccinimide powder was added directly to the enzyme/phosphate buffer solution, where it dissolved over the course of a few minutes.

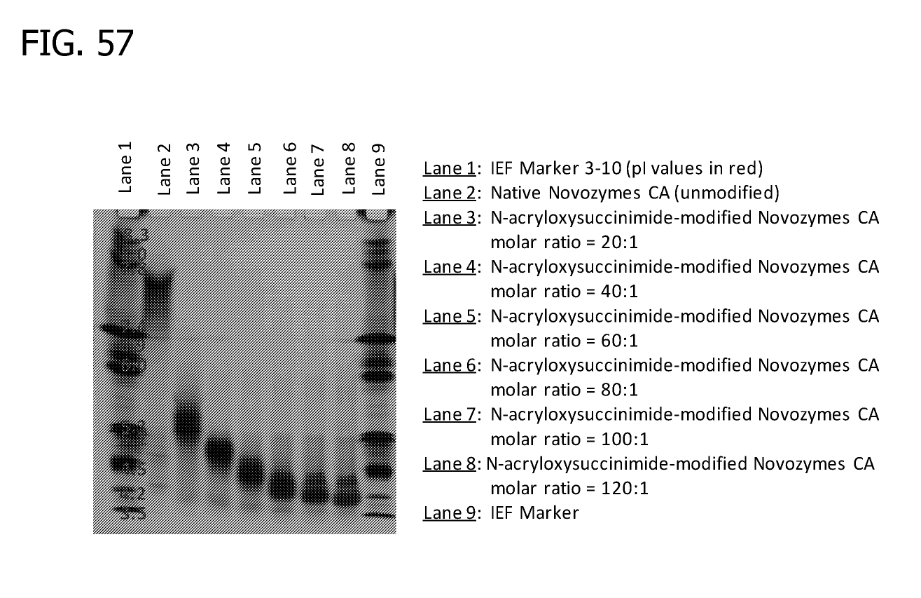

FIG. 57 shows the isoelectric focusing (IEF) gel electrophoresis results of a series of acryloxysuccinimide-functionalized Novozymes CA (Novozymes Item No. NS-81239 carbonic anhydrase, Lot No. 20100826). Since N-acryloxysuccinimide reacts with the amine groups present on the CA surface, a higher degree of functionalization results in a lower pI value, so the band shifts towards the bottom of the gel. Thus, as the molar ratio of N-acryloxysuccinimide to CA was increased, the enzyme became more functionalized with acrylamide groups.

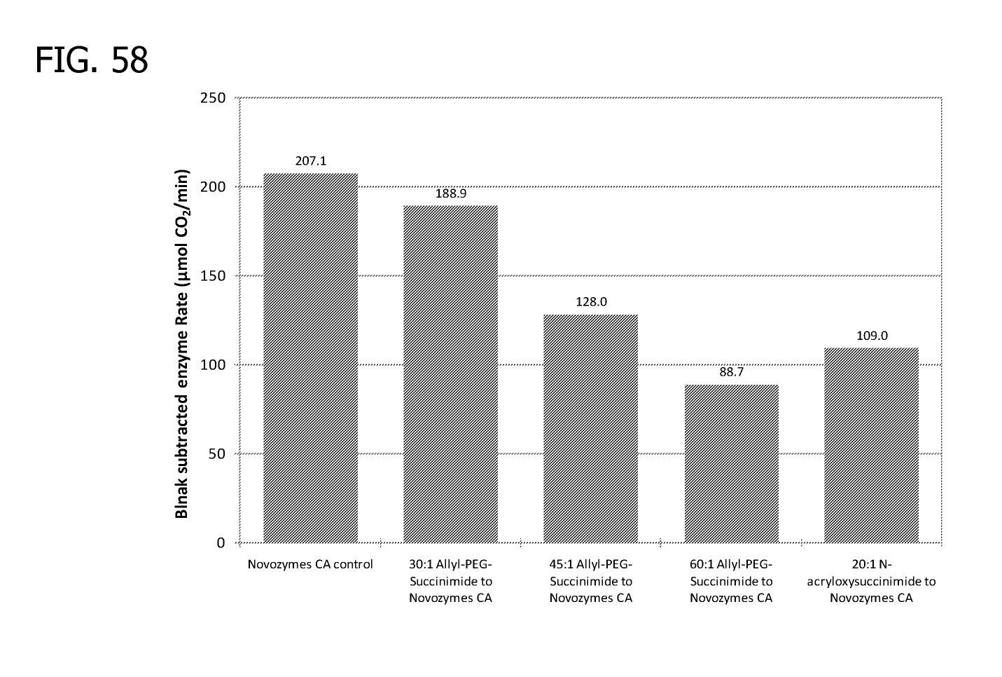

FIG. 58 shows the activity of vinyl-functionalized Novozymes CA using both allyl-PEG-succinimde and N-acryloxysuccinimide. As the extent of functionalization was increased, the overall enzyme activity decreased. However, a 45:1 molar excess of allyl-PEG-succinimide had >50% activity relative to the unmodified control.

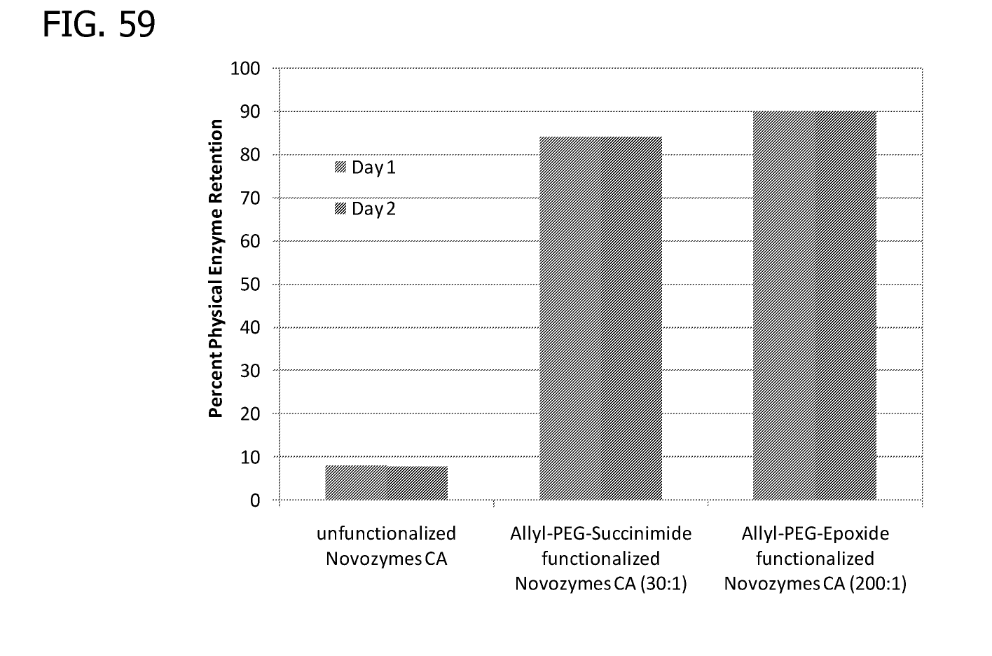

FIG. 59 shows the enzyme retention of the enzyme in a 55 micron thick PHMS-g-PEG micellar polymer coating (30 wt. % PEG overall) on functionalized ceramic as compared to unmodified Novozymes CA immobilized in the same material.

### Example 32

### Immobilization of CA in PHMS-g-PEG/PEG-Succinimide Micellar Polymer Coatings on Ceramic

A micellar polymer mixture was made with the following components: 2 mL of PHMS-g-PEG/PEG-Succinimide (76 wt. % PEG; 7 succinimides per PHMS chain), 2 mL of disilanol-terminated PDMS (4200 g/mol), 0.44 mL of PHMS (2250 g/mol), and 0.44 mL of a 50 wt. % solution of silanol trimethylsilyl-modified Q resin in decamethylcyclopentasiloxane. This micellar polymer mixture had 32 wt. % PEG overall.

A 100 mL glass beaker was filled with 1 mL of the above micellar polymer mixture and 0.4 mL of an aqueous Novozyme CA solution (˜300 mg/mL) and stirred thoroughly to mix. Bis(2-ethylhexanoate)tin cross-linking catalyst (80 μL) was stirred in quickly to mix, followed by 25 g of PDMS-functionalized Berl saddle ceramic packing pieces (˜180 pieces=30 mL; specific surface area of 6.28 cm²/cm³). The ceramic pieces were thoroughly stirred to coat them with the enzyme/polymer mixture and then spread out on a piece of stainless steel mesh to prevent pieces from adhering together during cross-linking. The polymer coated ceramic was allowed to cross-link for 2 hours at room temperature. This procedure was repeated three more times in order to obtain the 100 mL of polymer-coated packing required for batch reactor testing. The sample was equilibrated in 0.8M KHCO₃/1.2M K₂CO₃ overnight prior to analysis.

This sample had a 12 wt. % enzyme loading in the micellar polymer mixture. This enzyme loading could be changed in several ways. The amount of aqueous enzyme solution added to the micellar polymer mixture could be adjusted, with an upper limit of ˜30% (0.3 mL of aqueous enzyme solution to 1 mL micellar polymer mixture). Adding more water than that to the micellar polymer mixture hindered cross-linking and prevented good coating formation. Also, the concentration of the enzyme solution could be changed. Instead of using an aqueous enzyme solution, the enzyme could be added to the micellar polymer mixture as a powder before cross-linking into a coating.

Based on the measured transfer efficiency of ˜80% in the above method (meaning 80% of the enzyme/polymer mixture was actually coated onto the ceramic pieces) and the specific surface area of the ceramic, the film thickness of this coating was estimated to be ˜50 μm. This thickness could be easily adjusted by changing the volume/number of ceramic pieces added to the same amount of enzyme/micellar polymer mixture.

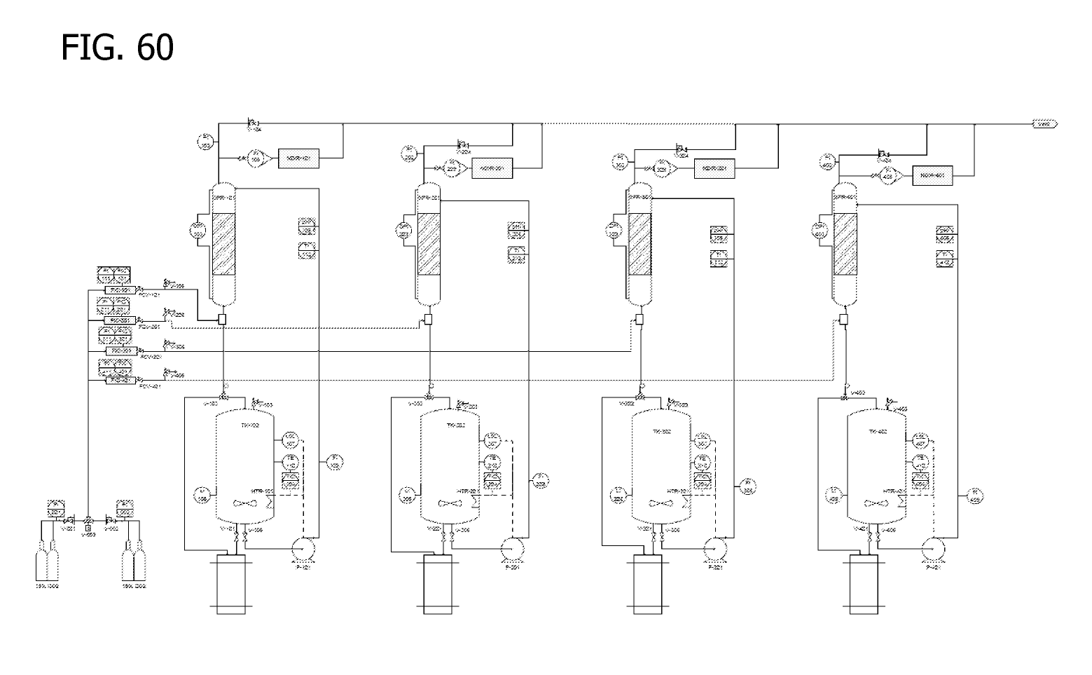

To test the immobilized enzyme activity of this sample, it was analyzed in a single pass reactor (SPR) configuration. The SPR analysis system functions as a small-scale absorber column and does not use a stripper column to regenerate solvent. Ceramic packing coated with immobilized enzyme/micellar polymer matrix were packed into a 78.5 cm tall×⅝″ i.d. counter-flow column, in which a 0.8M KHCO₃/1.2M K₂CO₃ solution (roughly pH=10) at room temperature was dripped at a rate of 20 mL/minutes from the top of the column and 15% CO₂ gas (balanced with nitrogen) flowed upwards from the bottom. The quantity of CO₂ gas at the output of the column was monitored by a non-dispersive infrared detector (NDIR), and the differential between the CO₂ content of the output gas vs. the feed gas was used to calculate the rate of absorption. A schematic of the single pass reactor is shown in FIG. 60.

A daily check was typically performed on the NDIR analyzer. If the analyzer did not meet satisfactory requirements then the system was recalibrated (15% CO₂ calibration). The reactor column contained a mesh screen to prevent the coated random packing pieces from passing all the way through the column. The column was secured in place with O-rings between the sanitary fittings at the top and bottom of the column. The column was secured in place using clamps at the top and bottom of the apparatus, and a liquid dispenser was inserted within the column as close to the center as possible. The CO₂ gas flow started and the desired pressure was established and the NDIR reached a steady state. The liquid flow was started, typically at a flow rate of 20 mL/min). Data was logged for the experiment using a pre-designed Labview program, while simultaneously logging readings from the NDIR, pH, and temperature by hand. Enzyme activity was calculated based on the magnitude of CO₂ conversion (i.e., CO₂ out mol %) and was reported as % conversion. Also reported was the overall mass transfer coefficient (i.e., K_{G} (mmol/s·m²·kPa)).

The sample described above was analyzed in the SPR system to determine immobilized enzyme activity. The results are summarized in Table 3. As seen in this table, this sample reached CO₂ conversions of 50% or more and was reproducible over multiple runs over multiple days, indicating that the enzyme was in fact successfully tethered into the micellar polymer film. These conversions represent an enhancement factor over blank polymer coated ceramic packing (no enzyme) in the 3-5 times range.

TABLE 3
SPR Activity Results of Novozymes CA Immobilized in PHMS-g-
PEG/PEG-Succinimide Micellar Polymer

| | | | | | | Rate |
|---|---|---|---|---|---|---|
| Sample | | | | Time | CO₂ % | Constant |
| ID | Date | Enzyme | Description | (min) | conversion | Multiplier |
| LW3-87 | Aug. 2, 2011 | Blank | blank PHMS-g-PEG (~30 wt % PEG) | T = 0 | 15.5% | 1.00 |
| | | | crosslinked with tin catalyst | T = 30 | 13.5% | 1.00 |
| CD 8-50B | Aug. 2, 2011 | unfunctionalized | 12 wt % CA in PHMS-g-PEG/PEG- | T = 0 | 53.5% | 4.60 |
| | | Novozymes CA | succinimide (~34 wt % PEG; high crosslink | T = 30 | 45.2% | 4.17 |
| | | | density; ~100:1 succinimide to CA) | | | |
| CD 8-50B | Aug. 2, 2011 | unfunctionalized | | T = 0 | 51.4% | 4.30 |
| | | Novozymes CA | | T = 30 | 39.9% | 3.51 |
| CD 8-50B | Aug. 5, 2011 | unfunctionalized | | T = 0 | 50.6% | 4.78 |
| | | Novozymes CA | | T = 30 | 37.0% | 3.10 |

### Example 33

### Immobilization of Allyl-PEG Functionalized CA in PHMS-g-PEG Micellar Polymer Coatings on Ceramic

### Example 33(a)

### Functionalized CA that Tethered into PHMS-g-PEG During Cross-Linking

A micellar polymer mixture was made with the following components: 3 mL of PHMS-g-PEG (62 wt. % PEG), 3 mL of PHMS-g-PEG (71 wt. % PEG), 3 mL of PHMS-g-PEG (82 wt. % PEG), 9 mL of disilanol-terminated PDMS (4200 g/mol), 2 mL of PHMS (2250 g/mol), and 2 mL of a 50 wt. % solution of silanol trimethylsilyl-modified Q resin in decamethylcyclopentasiloxane. This micellar polymer mixture had 29 wt. % PEG overall.

A 100 mL glass beaker was charged with 1.0 mL of the above micellar polymer mixture and 0.4 mL of an aqueous allyl-PEG functionalized Novozymes CA solution (˜200 mg/mL; 45:1 ratio of allyl-PEG-succinimide to CA during functionalization) and stirred thoroughly to mix. A solution of cross-linking catalyst platinum-divinyltetramethyldisiloxane complex in xylene (20 μL) was stirred in quickly to mix, followed by 32.0 g of allyl-functionalized ceramic spheres (nominally ⅛ inch diameter spheres; 132 g=˜100 mL of packing in SPR). The ceramic pieces were thoroughly stirred to coat them with the enzyme/polymer mixture and then spread out on a piece of stainless steel mesh to prevent pieces from adhering together during cross-linking. The mesh was then transferred into a 50° C. oven to allow the polymer coating to cross-link for 30 minutes. This procedure was repeated three more times to obtain the 50 mL of polymer-coated packing required for SPR testing. The sample was equilibrated in 0.8M KHCO₃/1.2M K₂CO₃ overnight prior to analysis.

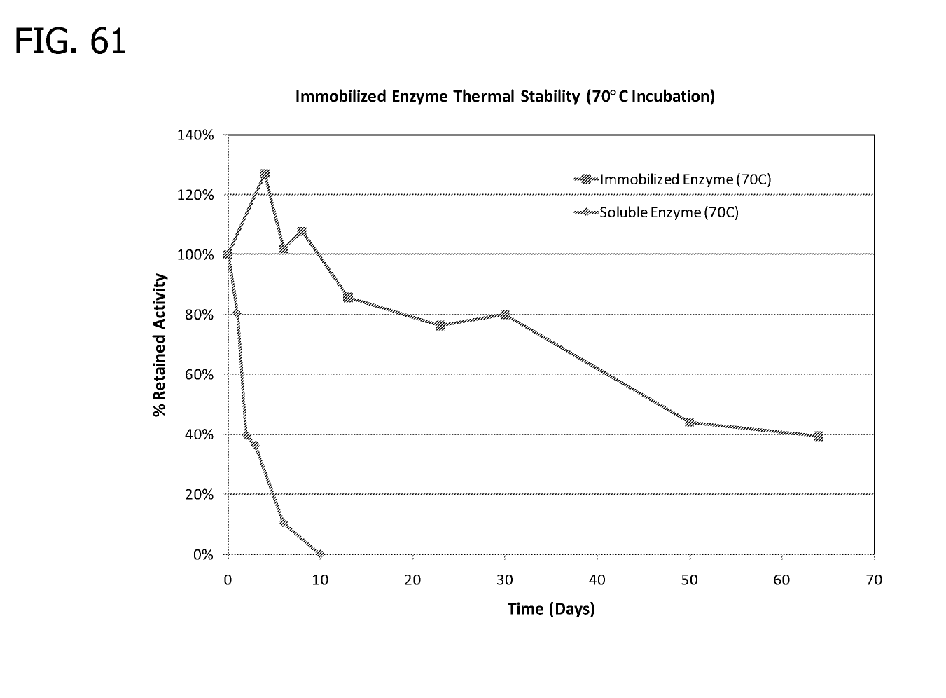

After equilibration and initial testing in the SPRs, this sample was divided evenly into two aliquots by weight, with one being kept at room temperature in 0.8M KHCO₃/1.2M K₂CO₃ and the other stored in 0.8M KHCO₃/1.2M K₂CO₃ at 70° C. Both samples were then periodically analyzed in the SPRs at room temperature after the 70° C. sample had sufficiently cooled. The activity of the elevated temperature sample was then compared to the one at room temperature to determine the thermal stability of the CA immobilized in the micellar polymer film. After analysis, both samples were returned to fresh 0.8M KHCO₃/1.2M K₂CO₃ solution at their respective storage temperatures until the next analysis. This data is shown in FIG. 61, comparing its thermal stability to that of a free enzyme solution made up at 1 mg/mL in 0.5M KHCO₃/0.5M K₂CO₃ and stored at 70° C. As seen from this figure, the half-life of soluble Novozymes CA at 70° C. is ˜2 days, whereas the half-life of immobilized Novozymes CA at 70° C. is ˜50 days. Immobilization of this isoform of CA in a PHMS-g-PEG micellar polymer thus improves its 70° C. half-life by a factor of 25, a significant improvement in its thermal stability.

### Example 33(b)

### Functionalized CA as the Only Cross-Linker

A 100 mL glass beaker was charged with 1.0 mL of PHMS-g-PEG (62 wt. % PEG) and 0.4 mL of an aqueous allyl-PEG functionalized Novozyme CA solution (˜210 mg/mL; 45:1 molar ratio of allyl-PEG-succinimide to CA during functionalization) and stirred thoroughly to mix. A solution of cross-linking catalyst platinum-divinyltetramethyldisiloxane complex in xylene (10 μL) was stirred in quickly to mix, followed by 33 g of allyl-functionalized 3 mm ceramic sphere packing pieces (˜66 g=50 mL; specific surface area of 7.16 cm²/g). The ceramic pieces were thoroughly stirred to coat them with the enzyme/polymer mixture and then spread out on a piece of stainless steel mesh to prevent pieces from adhering together during cross-linking. The polymer coated ceramic was allowed to cross-link for 30 minutes at 50° C. This procedure was repeated three more times to obtain 100 mL of polymer-coated packing for SPR testing. The sample was equilibrated in 0.8M KHCO₃/1.2M K₂CO₃ overnight prior to analysis. The SPR analysis of this sample is included as second entry in Table 33.1.

This sample had an 8.4 wt. % enzyme loading in the micellar polymer mixture. This enzyme loading could be changed in several ways. The amount of aqueous enzyme solution added to the micellar polymer mixture could be adjusted, but increased enzyme loading led to increased cross-linking density as the enzyme was the cross-linker. Also, the concentration of the enzyme solution could be changed.

Based on the measured transfer efficiency of ˜85% in the above method (meaning 85% of the enzyme/polymer mixture was actually coated onto the ceramic pieces) and the specific surface area of the ceramic, the film thickness of this coating was estimated to be ˜35 μm. This thickness could be easily adjusted by changing the volume/number of ceramic pieces added to the same amount of enzyme/micellar polymer mixture.

TABLE 33.1
SPR activity results of allyl-PEG Novozymes CA immobilized in PHMS-g-PEG
micellar polymers on 100 mL of ceramic packing

| | | | | K_{G} | Rate |
|---|---|---|---|---|---|
| | | Time | CO₂ % | (mmol/s * m² * | Constant |
| Enzyme | Description | (min) | conversion | kPa) | Multiplier |
| 45:1 allyl-PEG- | 6.4 wt % in PHMS-g-PEG(~37 wt % PEG; | T = 0 | 44.5% | 0.039 | 2.84 |
| succinimide funct. | enzyme + vinyl PDMS as cross-linkers) | T = 30 | 37.9% | 0.031 | 2.43 |
| Novozymes CA | crosslinked via Pt catalyst at 50 C | | | | |
| 45:1 allyl-PEG- | 8.4 wt % in PHMS-g-PEG(~60 wt % PEG; | T = 0 | 55.6% | 0.051 | 3.86 |
| succinimide funct. | enzyme only as cross-linker) crosslinked | T = 30 | 44.1% | 0.037 | 2.79 |
| Novozymes CA | via Pt catalyst at 50 C | | | | |
| 45:1 allyl-PEG- | 8.4 wt % in PHMS-g-PEG(~70 wt % PEG; | T = 0 | 64.8% | 0.067 | 4.94 |
| succinimide funct. | enzyme only as cross-linker) crosslinked | T = 30 | 56.2% | 0.053 | 4.12 |
| Novozymes CA | via Pt catalyst at 50 C | | | | |

Table 33.2 shows the SPR activity for a series of samples made with varying enzyme loading in a 70 wt. % PHMS-g-PEG micellar polymer and that there is a balance between enzyme loading and cross-linking density with respect to overall carbon dioxide capture rate.

TABLE 33.2
SPR activity results of allyl-PEG Novozymes CA immobilized in PHMS-g-PEG
micellar polymers on 50 mL of packing

| | | | | K_{G} | Rate |
|---|---|---|---|---|---|
| | | Time | CO₂ % | (mmol/s * m² * | Constant |
| Enzyme | Description | (min) | conversion | kPa) | Multiplier |
| 45:1 allyl-PEG- | 4.2 wt % in PHMS-g-PEG(~70 wt % PEG; low | T = 0 | 33.8% | 0.052 | 3.14 |
| succinimide funct. | crosslink density) crosslinked via Pt | T = 30 | 30.5% | 0.042 | 2.68 |
| Novo II | catalyst at 50 C | | | | |
| 45:1 allyl-PEG- | 8.4 wt % in PHMS-g-PEG(~70 wt % PEG; low | T = 0 | 38.6% | 0.061 | 3.45 |
| succinimide funct. | crosslink density) crosslinked via Pt | T = 30 | 34.1% | 0.052 | 2.94 |
| Novo II | catalyst at 50 C | | | | |
| 45:1 allyl-PEG- | 12.6 wt % in PHMS-g-PEG(~70 wt % PEG; | T = 0 | 49.4% | 0.085 | 4.85 |
| succinimide funct. | low crosslink density) crosslinked via Pt | T = 30 | 37.9% | 0.06 | 3.38 |
| Novo II | catalyst at 50 C | | | | |
| 45:1 allyl-PEG- | 21 wt % in PHMS-g-PEG(~70 wt % PEG; low | T = 0 | 34.6% | 0.053 | 3.03 |
| succinimide funct. | crosslink density) crosslinked via Pt | T = 30 | 30.4% | 0.046 | 2.58 |
| Novo II | catalyst at 50 C | | | | |
| 45:1 allyl-PEG- | 31.5 wt % in PHMS-g-PEG(~70 wt % PEG; | T = 0 | 33.2% | 0.05 | 2.87 |
| succinimide funct. | low crosslink density) crosslinked via Pt | T = 30 | 27.0% | 0.039 | 2.24 |
| Novo II | catalyst at 50 C | | | | |

### Example 34

### Immobilization of Allyl-PEG Functionalized CA in PHMS-g-PEG Micellar Polymer Coatings on Ceramic with the Incorporation of Charged Polymers

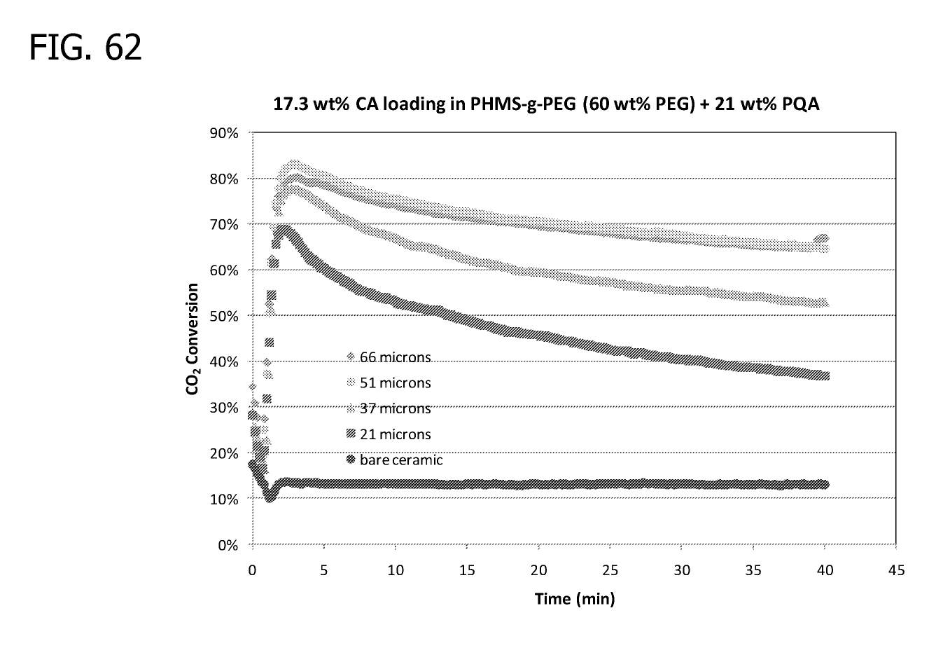

A 100 mL glass beaker was charged with 0.7 mL of PHMS-g-PEG (62 wt. % PEG) and 0.3 g of a 50 wt. % solution of Poly(diallyldimethylammonium chloride) 100,000-200,000 MW (PQA). The two polymers were mixed together, and then 0.7 mL of an aqueous Allyl-PEG functionalized Novozyme CA solution (˜210 mg/mL; 30:1 molar ratio of allyl-PEG-succinimide to CA during functionalization) was added and stirred thoroughly to mix. A solution of cross-linking catalyst platinum divinyl tetramethyl disiloxane complex in xylene (10 μL) was stirred in quickly to mix, followed by 33 g of allyl-functionalized 3 mm ceramic sphere packing pieces (˜66 g=50 mL; specific surface area of 7.16 cm²/g). The ceramic pieces were thoroughly stirred to coat them with the enzyme/polymer mixture and then spread out on a piece of stainless steel mesh to prevent pieces from adhering together during cross-linking. The coated ceramic was allowed to cross-link for 2 hours at room temperature. This procedure was repeated in order to obtain the 50 mL of polymer-coated packing required for batch reactor testing. The sample was equilibrated in 0.8M KHCO₃/1.2M K₂CO₃ overnight prior to analysis. The SPR analysis of this sample is included as the green line (37 micron film thickness) in FIG. 62.

This sample had a 14.7 wt. % enzyme loading in the micellar polymer mixture. This enzyme loading could be changed in several ways. The amount of aqueous enzyme solution added to the micellar polymer mixture could be adjusted, but increased enzyme loading led to increased cross-linking density as the enzyme was the cross-linker. Also, the concentration of the enzyme solution could be changed.

The sample had a 15 wt. % PQA loading in the micellar polymer mixture. This loading could be altered by adjusting the concentration of solution added, or by changing the volume of PQA solution added to the polymer mixture.

The ionic group utilized could also be altered, including the use of polyacrylic acids or ionic monomers, such as sulfobetaine methacrylic acid.

Based on the measured transfer efficiency of ˜85% in the above method (meaning 85% of the enzyme/polymer mixture was actually coated onto the ceramic pieces) and the specific surface area of the ceramic, the film thickness of this coating was estimated to be ˜37 μm. This thickness could be easily adjusted by changing the volume/number of ceramic pieces added to the same amount of enzyme/micellar polymer mixture.

Also shown in FIG. 62, when the film thickness was varied from 21 microns to 66 microns for a 17.3 wt % allyl-PEG CA loading in PHMS-g-PEG (60 wt % PEG)+21 wt % PQA, the single pass reactor data showing the CO₂ conversion under testing conditions of ˜50 mL of ceramic packing, 400 sccm of 15% CO₂ inlet, 20 mL/min of a 20 wt % carbonate solution at 25% conversion (˜0.8M KHCO₃/1.2M K₂CO₃), 1 psig, room temperature shows a maximum conversion rate at a film thickness of 51 microns.

### Example 35

### Immobilization of Allyl-PEG Functionalized CA in PHMS-g-PEG Micellar Polymer Coatings on Sulzer Structured Packing with the Incorporation of Charged Polymers

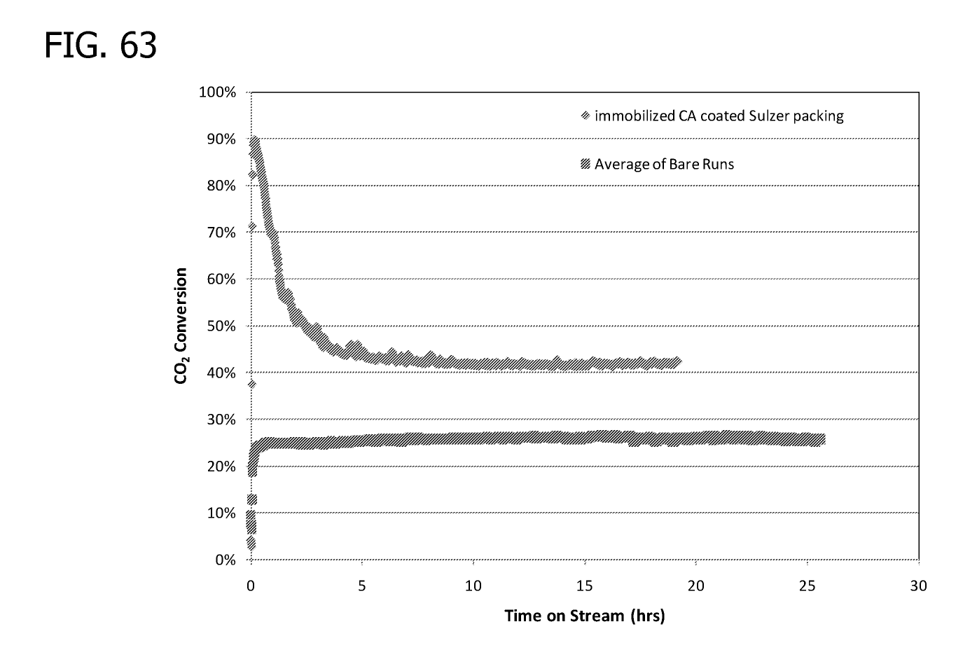

A 100 mL glass beaker was charged with 18 mL of PHMS-g-PEG (62 wt. % PEG) and 7.8 g of a 50 wt. % solution of Poly(diallyldimethylammonium chloride) 100,000-200,000 MW (PQA). The two polymers were mixed together, and then 18 mL of an aqueous Allyl-PEG functionalized Novozyme CA solution (˜210 mg/mL; 30:1 molar ratio of allyl-PEG-succinimide to CA during functionalization) was added along with 21.6 mL of reverse osmosis water (to decrease viscosity) and stirred thoroughly to mix. A platinum inhibitor, 270 μL of 3 methyl-1-butyn-3-ol, was added to the polymer-enzyme mixture, then a solution of cross-linking catalyst platinum divinyl tetramethyl disiloxane complex in xylene (270 μL) was stirred in quickly to mix. A piece of Sulzer structured stainless steel packing (17 pieces=1 L; surface area of 0.911 m²/L packing) was dipped on each side into the polymer-enzyme mixture, then the excess was allowed to drip off. The piece was rotated every few minutes to ensure even coating. In places where excess coating mixture had collected, compressed air was utilized to remove excess polymer mixture and maintain an even coating. The coated Sulzer was allowed to cross-link overnight at room temperature under a fan (to remove the volatile inhibitor). This procedure was repeated to obtain the number of pieces required for testing in the Closed Loop Reactor (CLR). The sample was equilibrated in 0.8M KHCO₃/1.2M K₂CO₃ for 3 hours prior to analysis. The CLR analysis of this sample is shown in FIG. 63 compared to that of an uncoated Sulzer packing blank run.

This sample had a 22.9 wt. % enzyme loading in the micellar polymer mixture. This enzyme loading could be changed in several ways. The amount of aqueous enzyme solution added to the micellar polymer mixture could be adjusted, but increased enzyme loading led to increased crosslinking density as the enzyme was the crosslinker. Also, the concentration of the enzyme solution could be changed.

The sample had a 15.2 wt. % PQA loading in the micellar polymer mixture. This loading could be altered by adjusting the concentration of solution added, or by changing the volume of PQA solution added to the overall mixture.

The film thickness could be easily adjusted by changing the amount of reverse osmosis water added to the enzyme/micellar polymer mixture.

### Example 36

### CA Isoforms

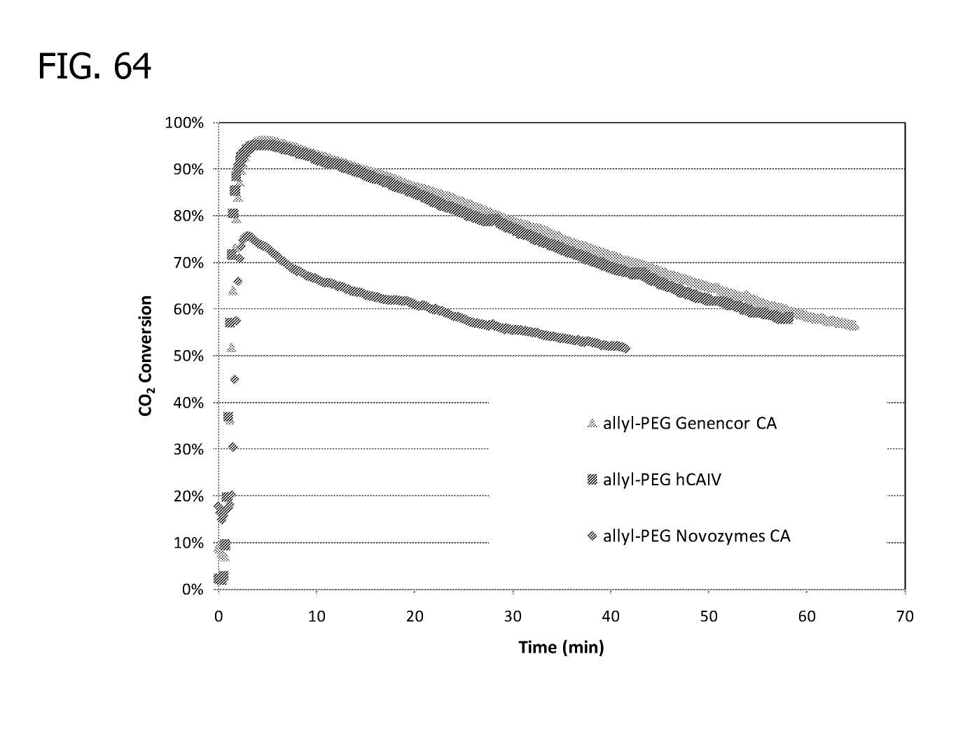

Alternate isoforms of CA were obtained from various suppliers to compare activities in the same immobilization matrix of PHMS-g-PEG (60 wt. % PEG) and 21 wt. % PQA (˜35 μm film thickness). FIG. 64 shows the SPR results for three different isoforms of CA in the same micellar polymer immobilization matrix. The samples made with hCAIV (supplied by Dr. Bill Sly at Saint Louis University) and CA supplied by Genencor (CRC 01993, carbonic anhydrase, Lot No. 11086301) outperformed the Novozymes CA sample. Using either of these two isoforms enabled peak CO₂ conversion rates as high as 95% and were better than the rates observed when using 4 g/L of free enzyme. This conversion corresponded to a rate constant multiplier of 20 times the blank for these immobilized enzymes.

In view of the above, it will be seen that the several objects of the invention are achieved and other advantageous results attained.

As various changes could be made in the above polymers, pharmaceutical compositions, and methods of treatment without departing from the scope of the invention, it is intended that all matter contained in the above description and shown in the accompanying drawing[s] shall be interpreted as illustrative and not in a limiting sense.

1. An enzyme immobilized by entrapment in a polymeric micellar or inverted micellar immobilization material and by covalent attachment of the enzyme to the polymeric micellar or inverted micellar immobilization material.

2. The immobilized enzyme of claim 1 wherein the polymeric micellar or inverted micellar immobilization material has discrete hydrophobic and hydrophilic regions in the solid state.

3. The immobilized enzyme of claim 2 wherein the polymeric micellar or inverted micellar immobilization material is a polysulfone, a polycarbonate, a poly(vinylbenzyl chloride), a polysiloxane, or a combination thereof.

4. The immobilized enzyme of claim 3 wherein the polymeric micellar or inverted micellar immobilization material comprises a polysiloxane.

5. The immobilized enzyme of claim 4 wherein the polysiloxane is a crosslinked polysiloxane reaction product derived from a crosslinking reaction mixture of a grafted polysiloxane and a polysiloxane comprising a vinyl or silanol group; wherein the grafted polysiloxane is derived from a grafting reaction mixture comprising a polysiloxane containing silicon hydride (Si—H) bonds and a hydrophilic group having a vinyl or allyl group.

6. The immobilized enzyme of claim 5 wherein the grafting reaction mixture further comprises a catalyst.

7. (canceled)

8. The immobilized enzyme of claim 5 wherein the crosslinking reaction mixture further comprises a catalyst.

9. (canceled)

10. The immobilized enzyme of claim 1 wherein the polymeric micellar or inverted micellar immobilization material has the structure of Formula 1: 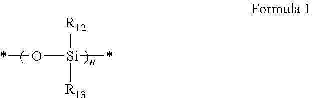
wherein
R₁₂ and R₁₃ are hydrogen, alkyl, —O—(SiR₁₄R₁₅—O)ₘ—; or —C₁ to C₁₀ alkyl, —C₁ to C₁₀ alkylene-acid, —C₁ to C₁₀ alkylene-base, or —C₁ to C₁₀ alkylene-enzyme, wherein the —CH₃ group or one or more of the —CH₂— groups can be replaced by an amine group, an oxygen, an amide group, or a carbonyl group or wherein the —CH₃ group or one or more of the —CH₂— groups is substituted with a hydroxy, alkyl, or alkoxy;
R₁₄ and R₁₅ are independently alkyl;
m and n are independently integers from 10 to 1000;
provided that the average number of —C₁ to C₁₀ alkylene-enzyme groups per repeat unit is at least 0.03.

11. The immobilized enzyme of claim 10 wherein R₁₂ and R₁₃ are independently hydrogen, alkyl, —(CH₂)_{q}O—(CH₂—CH₂—O)_{z}—Rₜ, —CH₂—O—(CH₂(CH₃)—CH₂—O)_{z}—Rₜ, or a combination thereof, wherein z is an integer corresponding to a weight average molecular weight of about 150 Da to about 8000 Da, Rₜ is hydrogen, alkyl, or enzyme.

12. The immobilized enzyme of claim 10 wherein when R₁₂ and R₁₃ are independently, hydrogen, —C₁ to C₁₀ alkylene-enzyme or Rₜ is enzyme, and at least one of R₁₂ or R₁₃ are —C₁ to C₁₀ alkylene-enzyme, the enzyme has at least one more link to the polymeric micellar or inverted micellar immobilization material.

13. The immobilized enzyme of claim 10 wherein the acid of the —C₁ to C₁₀ alkylene-acid or a salt thereof comprises a carboxylic, a phosphonic, a phosphoric, a sulfonic, a sulfuric, a sulfamic, or a combination thereof.

14. The immobilized enzyme of claim 10 wherein the base of the —C₁ to C₁₀ alkylene-base or a salt thereof comprises a tertiary amine, a quaternary amine, a nitrogen heterocycle, or a combination thereof.

15. The immobilized enzyme of claim 10 wherein R₁₂ and R₁₃ are independently hydrogen, alkyl, —(CH₂)₃—O—((CH₂)₂—O)_{z}—CH₃, —(CH₂)₂—C(O)—O—(CH₂)₂-imidazolium, or —(CH₂)₃—O—CH₂—CH(OH)—N(CH₃)—(CH₂)₂—SO₃Na, or —(CH₂)₃—O—(CH₂)₂—O)_{z}-enzyme.

16. The immobilized enzyme of claim 10 wherein m is an integer from 10 to 5000.

17.-18. (canceled)

19. The immobilized enzyme of claim 18 wherein the enzyme comprises a carbonic anhydrase.

20. The immobilized enzyme of claim 19 wherein the carbonic anhydrase is a cytosolic carbonic anhydrase, a mitochondrial carbonic anhydrase, a secreted carbonic anhydrase, or a membrane-associated carbonic anhydrase.

21.-22. (canceled)

23. The immobilized enzyme of claim 1 wherein the enzyme is covalently attached to the polymeric immobilization material through an amine, carboxylate, sulfhydryl, or hydroxyl group of the enzyme.

24.-37. (canceled)

38. The immobilized enzyme of claim 23 in the form of a pellet, a bead, a film, a coating, or a combination thereof.

39.-44. (canceled)

45. An enzyme having an amine group functionalized with a moiety, the moiety having the structure of formula 3 or formula 4 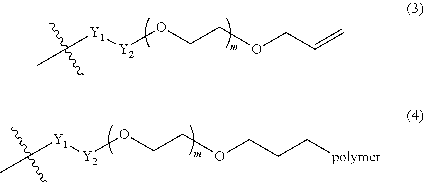
wherein m is an integer greater than 4, Y₁ is —CR₁₀R₁₁—, —O—, —S—, or —NR₁₂—, and Y₂ is —CCR₁₀R₁₁— or —C(O)—, R₁₀ and R₁₁ are independently hydrogen, alkyl, or aryl, and R₁₂ is hydrogen, alkyl, or aryl.

46.-61. (canceled)

62. An enzyme comprising an amine group functionalized with either a silsesquioxane; or a —C(O)—NH—R₁—Si(OH)₃ group wherein R₁ is —C₁ to C₁₀ alkylene or —C₁ to C₁₄ alkylene wherein one or more of the —CH₂— groups of the alkylene is replaced with an amine, an amide, or a carbonyl group.

63.-93. (canceled)
